(19) United States
(12) Patent Application Publication
(10) Pub. No.: US 2010/0184786 A1
(43) Pub. Date: Jul. 22, 2010

(54) Polar Quinazolines

(75) Inventors: Jeremy Mark Travins, Downington, PA (US); Ronald Charles Bernotas, Wayne, PA (US); Jay Edward Wrobel, Lawrenceville, NJ (US); David Harry Kaufman, Schwenksville, PA (US); Baihua Hu, Audubon, PA (US); James Winfield Jetter, Norristown, PA (US); David John O'Neill, Collegeville, PA (US); Charles William Mann, Plymouth Meeting, PA (US)

Correspondence Address: WYETH LLC;PATENT LAW GROUP, 5 GIRALDA FARMS, MADISON, NJ, 07940, US

(73) Assignee: Wyeth LLC, Madison, NJ (US)

(21) Appl. No.: 12/622,058

(22) Filed: Nov. 19, 2009

Related U.S. Application Data
(60) Provisional application No. 61/116,272, filed on Nov. 19, 2008.

(51) Int. Cl. A61K 31/517; C07D 239/74; A61P 9/00; A61P 3/04; A61P 3/06; A61P 3/10; A61P 29/00; A61Q 19/08; A61K 8/49

(52) U.S. Cl. 514/266.1; 544/283

(57) ABSTRACT

Disclosed are polar quinazoline-based modulators of Liver X receptors (LXRs) and related methods. The modulators include compounds of formula (I):

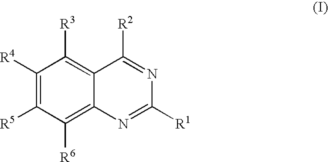

in which, R¹, R², R³, R⁴, R⁵, R⁶, R²², R²³, R²⁴, R²⁵, R²⁶, R²⁷, R²⁸, R²⁹, W, W¹, W², R^{a}, R^{b}, R^{c}, R^{d}, R^{e}, R^{f}, R^{g}, R^{h}, Rⁱ, R^{j}, R^{m}, Rⁿ, R^{o}, R^{p}, R^{q}, R^{r}, R^{s}, R^{t}, R^{u}, and n can be as defined anywhere herein. In general, these compounds can be used for treating or preventing one or more diseases, disorders, conditions or symptoms mediated by LXRs.

### TECHNICAL FIELD

This invention relates generally to polar quinazoline-based modulators of Liver X receptors (LXRs) and related methods.

### BACKGROUND

Atherosclerosis is among the leading causes of death in developed countries. Some of the independent risk factors associated with atherosclerosis include the presence of relatively high levels of serum LDL cholesterol and relatively low levels of serum HDL cholesterol in affected patients. As such, some anti-atherosclerotic therapy regimens include the administration of agents (e.g., statins) to reduce elevated serum LDL cholesterol levels.

Agents that increase patient HDL cholesterol levels can also be useful in anti-atherosclerotic therapy regimens. HDL cholesterol is believed to play a major role in the transport of cholesterol from peripheral tissues to the liver for metabolism and excretion (this process is sometimes referred to as “reverse cholesterol transport”). ABCA1 is a transporter gene involved in HDL production and reverse cholesterol transport. Upregulation of ABCA1 can therefore result in increased reverse cholesterol transport as well as inhibition of cholesterol absorption in the gut. In addition, HDL is also believed to inhibit the oxidation of LDL cholesterol, reduce the inflammatory response of endothelial cells, inhibit the coagulation pathway, and promote the availability of nitric oxide.

Liver X receptors (LXRs), originally identified in the liver as orphan receptors, are members of the nuclear hormone receptor super family and are believed to be involved in the regulation of cholesterol and lipid metabolism. LXRs are ligand-activated transcription factors and bind to DNA as obligate heterodimers with retinoid X receptors. While LXRα is generally found in tissues such as liver, kidney, adipose tissue, intestine and macrophages, LXRβ displays a ubiquitous tissue distribution pattern. Activation of LXRs by oxysterols (endogenous ligands) in macrophages results in the expression of several genes involved in lipid metabolism and reverse cholesterol transport including the aforementioned ABCA1; ABCG1; and ApoE. See, e.g., Koldamova, et al., J. Biol. Chem. 2003, 278, 13244.

Studies have been conducted in LXRα knock-out (k/o), LXRβ k/o and double k/o mice to determine the physiological role of LXRs in lipid homeostasis and atherosclerosis. The data from these studies suggested that in double k/o mice on normal chow diet, increased cholesterol accumulation was observed in macrophages (foam cells) of the spleen, lung and arterial wall. The increased cholesterol accumulation was believed to be associated with the presence of reduced serum HDL cholesterol and increased LDL cholesterol, even though the total cholesterol levels in the mice were about normal. While LXRα k/o mice did not appear to show significant changes in hepatic gene expression, LXRβ k/o mice showed 58% decrease in hepatic ABCA1 expression and 208% increase in SREBP1c expression suggesting that LXRβ may be involved in the regulation of liver SREBP1c expression.

Data obtained from studies employing two different atherosclerotic mouse models (ApoE k/o and LDLR k/o) suggest that agonists of LXRα or β can be relatively effective in upregulating ABCA1 expression in macrophages. For example, inhibition of atherosclerotic lesions could be observed when ApoE k/o and LDLR k/o mice were treated with LXRα or β agonists for 12 weeks. The tested agonists were observed to have variable effects on serum cholesterol and lipoprotein levels and appeared to cause a relatively significant increase in serum HDL cholesterol and triglyceride levels. These in vivo data were found to be consistent with in vitro data obtained for the same agonists in macrophages.

In addition to the lipid and triglyceride effects described above, it is also believed that activation of LXRs results in the inhibition of inflammation and proinflammatory gene expression. This hypothesis is based on data obtained from studies employing three different models of inflammation (LPS-induced sepsis, acute contact dermatitis of the ear and chronic atherosclerotic inflammation of the artery wall). These data suggest that LXR modulators can mediate both the removal of cholesterol from the macrophages and the inhibition of vascular inflammation.

For a review of LXR biology and LXR modulators, see, e.g., Goodwin, et al., Current Topics in Medicinal Chemistry 2008, 8, 781; and Bennett, et al., Current Medicinal Chemistry 2008, 15, 195.

For studies related to atherosclerosis, see, e.g., Scott, J. N. Engl. J. Med. 2007, 357, 2195; Joseph, et al., PNAS 2002, 99, 7604; Tangirala, et. al., PNAS, 2002, 99, 11896; and Bradley, et al., Journal of Clinical Investigation 2007, 117, 2337-2346.

For studies related to inflammation, see, e.g., Fowler, et al., Journal of Investigative Dermatology 2003, 120, 246; and US 2004/0259948.

For studies related to Alzheimer's disease, see, e.g., Koldamova, et al., J. Biol. Chem. 2005, 280, 4079; Sun, et al., J. Biol. Chem. 2003, 278, 27688; and Riddell, et al., Mol. Cell Neurosci. 2007, 34, 621.

For studies related to diabetes, see, e.g., Kase, et al., Diabetologia 2007, 50, 2171; and Liu, et al., Endocrinology 2006, 147, 5061.

For studies related to skin aging, see, e.g., WO 2004/076418; WO 2004/103320; and US 2008/0070883.

For studies related to arthritis, see, e.g., Chintalacharuvu, et. al., Arthritis a & Rheumatism 2007, 56, 1365; and WO 2008/036239.

### SUMMARY

This invention relates generally to polar quinazoline-based modulators of Liver X receptors (LXRs) and related methods.

In one aspect, this invention features a compound having formula (I):

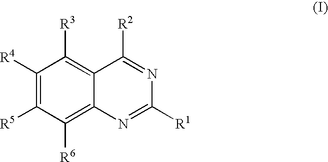

in which:

R¹ is hydrogen or C₁-C₂ alkyl;

R² has the following formula:

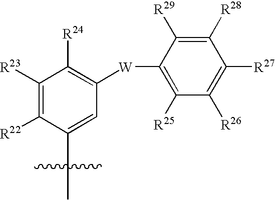

wherein:

each of R²², R²³, and R²⁴ is, independently, hydrogen or R^{a};

W is a bond, —O—, —NH—, —N(C₁-C₃ alkyl)-, C₁₋₂ alkylene, C₂ alkenylene, C₂ alkynylene, —(C₁₋₂ alkylene)O—, or —O(C₁₋₂ alkylene)-;

wherein all of R²⁵, R²⁶, R²⁷,R²⁸, and R²⁹ are defined according to either (A) or (B) below:

(A):

one of R²⁵, R²⁶, R²⁷, R²⁸, and R²⁹ is —W¹—S(O)ₙR^{b} or —W¹—S(O)ₙNR^{c}R^{d}; and the others are each, independently, hydrogen or R^{e};

or

(B):

(a) one of R²⁵, R²⁶, R²⁷, R²⁸, and R²⁹ is —W¹—S(O)ₙR^{f}; and

(b) one of R²⁵, R²⁶, R²⁷, R²⁸, and R²⁹ is —W¹—C(O)OR^{g}, —W¹—C(O)NR^{h}Rⁱ; or —W²—CN; and

(c) the others are each, independently, hydrogen or R^{e};

each of R³, R⁴ and R⁵ is, independently:

(i) hydrogen; or

(ii) halo; or

(iii) C₁-C₃ alkyl or C₁-C₃ haloalkyl, each of which is optionally substituted with from 1-2 R^{j};

R⁶ is:

(i) halo; or

(ii) C₁-C₆ alkyl or C₁-C₆ haloalkyl, each of which is optionally substituted with from 1-3 R^{j};

or

(iii) nitro; hydroxy; C₁-C₆ alkoxy; C₁-C₆ haloalkoxy; C₁-C₆ thioalkoxy; C₁-C₆ thiohaloalkoxy; cyano; or S(O)ₙR^{f};

wherein:

W¹ is a bond, —NH—, —N(C₁-C₃ alkyl)-, or C₁₋₂ alkylene;

W² is a bond or C₁₋₂ alkylene;

R^{a} at each occurrence is, independently, C₁-C₃ alkyl; C₁-C₃ haloalkyl; halo; hydroxy; NR^{m}Rⁿ; C₁-C₃ alkoxy; or C₁-C₃ haloalkoxy;

R^{b} is C₁-C₆ alkyl or C₁-C₆ haloalkyl, each of which is substituted with from 1-2 R^{o};

each of R^{c} and R^{d} at each occurrence is, independently:

(i) hydrogen; or

(i) C₁-C₆ alkyl or C₁-C₆ haloalkyl, each of which is optionally substituted with from 1-3 R^{j}; or

(ii) C₂-C₆ alkenyl or C₂-C₆ alkynyl, each of which is optionally substituted with from 1-3 R^{p}; or

(iii) C₃-C₈ cycloalkyl, C₃-C₈ cycloalkenyl, heterocyclyl including 3-8 atoms, heterocycloalkenyl including 3-10 atoms, C₇-C₁₁ aralkyl, or heteroaralkyl including 6-11 atoms, each of which is optionally substituted with from 1-3 R^{q}; or

(iv) C₆-C₁₀ aryl or heteroaryl including 5-10 atoms, each of which is optionally substituted with from 1-3 R^{r}; or

R^{c} and R^{d} together with the nitrogen atom to which they are attached form a heterocyclyl including 3-8 atoms or a heterocycloalkenyl including 3-10 atoms, each of which is optionally substituted with from 1-3 R^{q};

R^{e} at each occurrence is, independently, C₁-C₃ alkyl; C₁-C₃ haloalkyl; halo; hydroxy; NR^{m}Rⁿ; C₁-C₃ alkoxy; or C₁-C₃ haloalkoxy;

R^{f} at each occurrence is, independently C₁-C₃ alkyl or C₁-C₃ haloalkyl;

R^{g} is:

(i) hydrogen; or

(i) C₁-C₆ alkyl or C₁-C₆ haloalkyl, each of which is optionally substituted with from 1-3 R^{j};

each of R^{h} and Rⁱ at each occurrence is, independently:

(i) hydrogen; or

(i) C₁-C₃ alkyl or C₁-C₃ haloalkyl, each of which is optionally substituted with from 1-2 R^{j};

R^{j} at each occurrence is, independently, NR^{m}Rⁿ; hydroxy; C₁-C₆ alkoxy or C₁-C₆ haloalkoxy;

each of R^{m} and Rⁿ at each occurrence is, independently, hydrogen, C₁-C₆ alkyl, or C₁-C₆ haloalkyl;

R^{o} at each occurrence is, independently:

(i) —C(O)NR^{t}R^{u}; or

(ii) —C(O)OR^{s}; or

(iii) NHS(O)ₙR^{f} or —N(C₁-C₃ alkyl)S(O)ₙR^{f}; or

(iv) —S(O)ₙR^{f}; or

(v) —CN; or

(vi) —NR^{m}Rⁿ;

R^{p} at each occurrence is, independently:

(i) halo; NR^{m}Rⁿ; hydroxy; C₁-C₆ alkoxy or C₁-C₆ haloalkoxy; or

(ii) C₃-C₇ cycloalkyl optionally substituted with from 1-3 substituents independently selected

from NR^{m}Rⁿ; hydroxy; C₁-C₆ alkyl, C₁-C₆ alkoxy and C₁-C₆ haloalkoxy;

R^{q} at each occurrence is, independently:

(i) halo; NR^{m}Rⁿ; hydroxy; C₁-C₆ alkoxy or C₁-C₆ haloalkoxy; or

(ii) C₁-C₆ alkyl or C₁-C₆ haloalkyl; or

(iii) c₂-c₆ alkenyl or C₂-C₆ alkynyl;

R^{r} at each occurrence is, independently:

(i) halo; NR^{m}Rⁿ; hydroxy; C₁-C₆ alkoxy or C₁-C₆ haloalkoxy; or cyano; or

(ii) C₁-C₆ alkyl or C₁-C₆ haloalkyl, each of which is optionally substituted with from 1-3 R^{j}; or

(iii) C₂-C₆ alkenyl or C₂-C₆ alkynyl, each of which is optionally substituted with from 1-3 R^{p};

R^{s} is:

(i) hydrogen; or

(i) Cₗ-C₆ alkyl or C₁-C₆ haloalkyl, each of which is optionally substituted with from 1-3 R^{j};

each of R^{t} and R^{u} at each occurrence is, independently:

(i) hydrogen; or

(i) Cₗ-C₃ alkyl or Cₗ-C₃ haloalkyl, each of which is optionally substituted with from 1-2 R^{j}; and

n is 1 or 2;

or an N-oxide and/or a pharmaceutically acceptable salt thereof.

In one aspect, this invention relates to any subgenera of formula (I) described herein.

In one aspect, this invention relates to any of the specific polar quinazoline compounds delineated herein. In some embodiments, the compound of formula (I) can be selected from the title compounds of Examples 1-11, 14-23, 25-29, 31-57, 59 and 61-65; or a pharmaceutically acceptable salt and/or N-oxide thereof.

In one aspect, this invention features a composition (e.g., a pharmaceutical composition), which includes a compound of formula (I) (including any subgenera or specific compounds thereof), or a salt (e.g., a pharmaceutically acceptable salt), or an N-oxide, or a prodrug thereof and a pharmaceutically acceptable carrier. In some embodiments, the composition can include an effective amount of the compound or the salt thereof. In some embodiments, the composition can further include an additional therapeutic agent.

In one aspect, this invention features a dosage form, which includes from about 0.05 milligrams to about 2,000 milligrams (e.g., from about 0.1 milligrams to about 1,000 milligrams, from about 0.1 milligrams to about 500 milligrams, from about 0.1 milligrams to about 250 milligrams, from about 0.1 milligrams to about 100 milligrams, from about 0.1 milligrams to about 50 milligrams, or from about 0.1 milligrams to about 25 milligrams) of formula (I) (including any subgenera or specific compounds thereof), or a salt (e.g., a pharmaceutically acceptable salt), or an N-oxide, or a prodrug thereof. The dosage form can further include a pharmaceutically acceptable carrier and/or an additional therapeutic agent.

The invention also relates generally to modulating (e.g., activating) LXRs with the quinazoline compounds described herein. In some embodiments, the methods can include, e.g., contacting an LXR in a sample (e.g., a tissue, a cell free assay medium, a cell-based assay medium) with a compound of formula (I) (including any subgenera or specific compounds thereof) or a salt (e.g., a pharmaceutically acceptable salt), or an N-oxide, or a prodrug thereof. In other embodiments, the methods can include administering a compound of formula (I) (including any subgenera or specific compounds thereof) or a salt (e.g., a pharmaceutically acceptable salt), or an N-oxide, or a prodrug thereof to a subject (e.g., a mammal, e.g., a human, e.g., a human having or at risk of having one or more of the diseases or disorders described herein).

In one aspect, this invention also relates generally to methods of treating (e.g., controlling, relieving, ameliorating, alleviating, slowing the progression of, delaying the onset of, or reducing the risk of developing) or preventing one or more LXR-mediated diseases or disorders in a subject (e.g., a subject in need thereof). The methods include administering to the subject an effective amount of a compound of formula (I) (including any subgenera or specific compounds thereof) or a salt (e.g., a pharmaceutically acceptable salt), or an N-oxide, or a prodrug thereof. LXR-mediated diseases or disorders can include, e.g., cardiovascular diseases (e.g., acute coronary syndrome, restenosis, or coronary artery disease), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging or connective tissue diseases.

In another aspect, this invention relates to methods of modulating (e.g., increasing) serum HDL cholesterol levels in a subject (e.g., a subject in need thereof), which includes administering to the subject an effective amount of a compound of formula (I) (including any subgenera or specific compounds thereof) or a salt (e.g., a pharmaceutically acceptable salt), or an N-oxide, or a prodrug thereof.

In another aspect, this invention relates to methods of modulating (e.g., decreasing) serum LDL cholesterol levels in a subject (e.g., a subject in need thereof), which includes administering to the subject an effective amount of a compound of formula (I) (including any subgenera or specific compounds thereof) or a salt (e.g., a pharmaceutically acceptable salt), or an N-oxide, or a prodrug thereof.

In another aspect, this invention relates to methods of modulating (e.g., increasing) reverse cholesterol transport in a subject (e.g., a subject in need thereof), which includes administering to the subject an effective amount of a compound of formula (I) (including any subgenera or specific compounds thereof) or a salt (e.g., a pharmaceutically acceptable salt), or an N-oxide, or a prodrug thereof.

In another aspect, this invention relates to methods of modulating (e.g., decreasing or inhibiting) cholesterol absorption in a subject (e.g., a subject in need thereof), which includes administering to the subject an effective amount of a compound of formula (I) (including any subgenera or specific compounds thereof) or a salt (e.g., a pharmaceutically acceptable salt), or an N-oxide, or a prodrug thereof.

In a further aspect, this invention relates to methods of preventing or treating a cardiovascular disease (e.g., acute coronary syndrome, restenosis, or coronary artery disease), which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In one aspect, this invention relates to methods of preventing or treating atherosclerosis and/or atherosclerotic lesions, which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In another aspect, this invention relates to methods of preventing or treating diabetes (e.g., type I diabetes or type II diabetes), which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In a further aspect, this invention relates to methods of preventing or treating Syndrome X, which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In one aspect, this invention relates to methods of preventing or treating obesity, which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In another aspect, this invention relates to methods of preventing or treating a lipid disorder (e.g., one or more of dyslipidemia, hyperlipidemia, hypertriglyceridemia, hypercholesterolemia, low HDL and/or high LDL), which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In a further aspect, this invention relates to methods of preventing or treating a cognitive disorder (e.g., Alzheimer's disease or dementia), which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In one aspect, this invention relates to methods of preventing or treating dementia, which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In another aspect, this invention relates to methods of preventing or treating Alzheimer's disease, which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In a further aspect, this invention relates to methods of preventing or treating an inflammatory disease (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In another aspect, this invention relates to methods of preventing or treating rheumatoid arthritis, which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In a further aspect, this invention relates to methods of preventing or treating celiac, which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating thyroiditis, which includes administering to a subject in need thereof an effective amount of a compound of formula (I) (including any subgenera or specific compounds thereof) or a salt (e.g., a pharmaceutically acceptable salt), or an N-oxide, or a prodrug thereof.

In one aspect, this invention relates to methods of treating a connective tissue disease (e.g., osteoarthritis or tendonitis), which includes administering to a subject (e.g., a mammal, e.g., a human) in need thereof an effective amount of a compound of formula (I) (including any subgenera or specific compounds thereof) or a salt (e.g., a pharmaceutically acceptable salt), or an N-oxide, or a prodrug thereof. In embodiments, the compound of formula (I) inhibits (e.g., reduces or otherwise diminishes) cartilage degradation. In embodiments, the compound of formula (I) induces (e.g., increases or otherwise agments) cartilage regeneration. In embodiments, the compound of formula (I) inhibits (e.g., reduces or otherwise diminishes) cartilage degradation and induces (e.g., increases or otherwise agments) cartilage regeneration. In embodiments, the compound of formula (I) inhibits (e.g., reduces or otherwise diminishes) aggrecanase activity. In embodiments, the compound of formula (I) inhibits (e.g., reduces or otherwise diminishes) elaboration of pro-inflammatory cytokines in osteoarthritic lesions.

In another aspect, this invention relates to methods of treating or preventing skin aging, the method comprising administering (e.g., topically administering) to a subject (e.g., a mammal, e.g., a human) in need thereof an effective amount of a compound of formula (I) (including any subgenera or specific compounds thereof) or a salt (e.g., a pharmaceutically acceptable salt), or an N-oxide, or a prodrug thereof. In embodiments, the skin aging can be derived from chronological aging, photoaging, steroid-induced skin thinning, or a combination thereof.

The term “skin aging” includes conditions derived from intrinsic chronological aging (for example, deepened expression lines, reduction of skin thickness, inelasticity, and/or unblemished smooth surface), those derived from photoaging (for example, deep wrinkles, yellow and leathery surface, hardening of the skin, elastosis, roughness, dyspigmentations (age spots) and/or blotchy skin), and those derived from steroid-induced skin thinning. Accordingly, another aspect is a method of counteracting UV photodamage, which includes contacting a skin cell exposed to UV light with an effective amount of a compound of formula (I).

In some embodiments, the compound of formula (I) (including any subgenera or specific compounds thereof) or a salt (e.g., a pharmaceutically acceptable salt), or an N-oxide, or a prodrug thereof does not substantially increase serum and/or hepatic triglyceride levels of the subject.

In some embodiments, the administered compound of formula (I) (including any subgenera or specific compounds thereof) or a salt (e.g., a pharmaceutically acceptable salt), or an N-oxide, or a prodrug thereof can be an LXR agonist (e.g., an LXRα agonist or an LXRβ agonist).

In some embodiments, the subject can be a subject in need thereof (e.g., a subject identified as being in need of such treatment). Identifying a subject in need of such treatment can be in the judgment of a subject or a health care professional and can be subjective (e.g. opinion) or objective (e.g. measurable by a test or diagnostic method). In some embodiments, the subject can be a mammal. In certain embodiments, the subject is a human.

In a further aspect, this invention also relates to methods of making compounds described herein. Alternatively, the method includes taking any one of the intermediate compounds described herein and reacting it with one or more chemical reagents in one or more steps to produce a compound described herein.

In one aspect, this invention relates to a packaged product. The packaged product includes a container, one of the aforementioned compounds in the container, and a legend (e.g., a label or an insert) associated with the container and indicating administration of the compound for treatment and control of the diseases or disorders described herein.

In embodiments, any compound, composition, or method can also include any one or more of the features below (alone or in combination) and/or delineated in the detailed description and/or in the claims.

In general, the compounds of formula (I) have a Topological Polar Surface Area (“TPSA”) of greater than about 80 Å². TPSA refers to the sum of surfaces of polar atoms (typically oxygens, nitrogens and attached hydrogens) in a molecule as determined by summation of tabulated surface contributions of polar fragments (atoms regarding also their environment). Methods for calculating TPSA are known in the art, see, e.g., Ertl, P., Rohde, B., Selzer, P. “Fast calculation of molecular polar surface area as a sum of fragment based contributions and its application to the prediction of drug transport properties.” J. Med. Chem. 2000, 43: 3714-3717. While not wishing to be bound by theory, it is believed that compounds having a (TPSA) of greater than about 80 Å² have a reduced likelihood of penetrating the brain (see, e.g., Gleeson, J. Med. Chem. 2008, 51, 817), which can potentially reduce the CNS-related side effects.

In some embodiments, the compounds of formula (I) can have a TPSA of from about 100 Å² to about 120 Å².

In some embodiments, the compounds of formula (I) can have TPSA of from about 100 Å² to about 109 Å².

In some embodiments, the compounds of formula (I) can have TPSA of from about 110 Å² to about 120 Å².

In some embodiments, the compounds of formula (I) can have TPSA of from about 90 Å² to about 99 Å².

The term “mammal” includes organisms, which include mice, rats, cows, sheep, pigs, rabbits, goats, horses, monkeys, dogs, cats, and humans.

“An effective amount” refers to an amount of a compound that confers a therapeutic effect (e.g., treats, inhibits, controls, relieves, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect). An effective amount of the compound described above may range from about 0.01 mg/kg to about 1000 mg/kg, (e.g., from about 0.1 mg/kg to about 100 mg/kg, from about 1 mg/kg to about 100 mg/kg). Effective doses will also vary depending on route of administration, as well as the possibility of co-usage with other agents.

The term “halo” or “halogen” refers to any radical of fluorine, chlorine, bromine or iodine.

In general, and unless otherwise indicated, substituent (radical) prefix names are derived from the parent hydride by either (i) replacing the “ane” in the parent hydride with the suffixes “yl,” “diyl,” “triyl,” “tetrayl,” etc.; or (ii) replacing the “e” in the parent hydride with the suffixes “yl,” “diyl,” “triyl,” “tetrayl,” etc. (here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout. Conventional numbering/lettering systems are also adhered to for substituent numbering and the nomenclature of fused, bicyclic, tricyclic, polycyclic rings.

The term “alkyl” refers to a saturated hydrocarbon chain that may be a straight chain or branched chain, containing the indicated number of carbon atoms. For example, C₁-C₆ alkyl indicates that the group may have from 1 to 6 (inclusive) carbon atoms in it. Any atom can be optionally substituted, e.g., by one or more subsitutents. Examples of alkyl groups include without limitation methyl, ethyl, n-propyl, isopropyl, and tert-butyl.

The terms “alkylene,” “alkenylene,” and“alkynylene,” refer to divalent straight chain or branched chain alkyl (e.g., —CH₂—), alkenyl (e.g., —CH═CH—), and alkynyl (e.g., —C≡C—), respectively.

The term “haloalkyl” refers to an alkyl group, in which at least one hydrogen atom is replaced by halo. In some embodiments, more than one hydrogen atom (e.g., 2, 3, 4, 5, 6, 7, 8, 9, 10, 11, 12, 13, or 14) are replaced by halo. In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halo (sometimes referred to herein as perhaloalkyl, e.g., perfluoroalkyl, such as trifluoromethyl). Any atom can be optionally substituted, e.g., by one or more substituents.

The term “aralkyl” refers to an alkyl moiety in which an alkyl hydrogen atom is replaced by an aryl group. One of the carbons of the alkyl moiety serves as the point of attachment of the aralkyl group to another moiety. Any ring or chain atom can be optionally substituted e.g., by one or more substituents. Non-limiting examples of “aralkyl” include benzyl, 2-phenylethyl, and 3-phenylpropyl groups.

The term “heteroaralkyl” refers to an alkyl moiety in which an alkyl hydrogen atom is replaced by a heteroaryl group. One of the carbons of the alkyl moiety serves as the point of attachment of the aralkyl group to another moiety. Heteroaralkyl includes groups in which more than one hydrogen atom on an alkyl moiety has been replaced by a heteroaryl group. Any ring or chain atom can be optionally substituted e.g., by one or more substituents. Heteroaralkyl can include, for example, 2-pyridylethyl.

The term “alkenyl” refers to a straight or branched hydrocarbon chain containing the indicated number of carbon atoms and having one or more carbon-carbon double bonds. Any atom can be optionally substituted, e.g., by one or more substituents. Alkenyl groups can include, e.g., allyl, 1-butenyl, and 2-hexenyl. One of the double bond carbons can optionally be the point of attachment of the alkenyl substituent. The term “alkynyl” refers to a straight or branched hydrocarbon chain containing the indicated number of carbon atoms and having one or more carbon-carbon triple bonds. Any atom can be optionally substituted, e.g., by one or more substituents. Alkynyl groups can include, e.g., ethynyl, propargyl, and 3-hexynyl. One of the triple bond carbons can optionally be the point of attachment of the alkynyl substituent.

The term “alkoxy” refers to an —O-alkyl radical. The term “mercapto” refers to an SH radical. The term “thioalkoxy” refers to an —S-alkyl radical. The terms “aryloxy” and “heteroaryloxy” refer to an —O-aryl radical and —O-heteroaryl radical, respectively. The terms “thioaryloxy” and “thioheteroaryloxy” refer to an —S-aryl radical and —S-heteroaryl radical, respectively.

The terms “aralkoxy” and “heteroaralkoxy” refer to an —O-aralkyl radical and —O-heteroaralkyl radical, respectively. The terms “thioaralkoxy” and “thioheteroaralkoxy” refer to an —S-aralkyl radical and —S-heteroaralkyl radical, respectively. The term “cycloalkoxy” refers to an —O-cycloalkyl radical. The terms “cycloalkenyloxy” and “heterocycloalkenyloxy” refer to an —O-cycloalkenyl radical and —O-heterocycloalkenyl radical, respectively. The term “heterocyclyloxy” refers to an —O-heterocyclyl radical. The term “thiocycloalkoxy” refers to an —S-cycloalkyl radical. The terms “thiocycloalkenyloxy” and “thioheterocycloalkenyloxy” refer to an —S-cycloalkenyl radical and —S-heterocycloalkenyl radical, respectively. The term “thioheterocyclyloxy” refers to an —S-heterocyclyl radical.

The term “heterocyclyl” refers to a fully saturated monocyclic, bicyclic, tricyclic or other polycyclic ring system having one or more (e.g., 1-4) heteroatom ring atoms independently selected from O, N, or S. The heteroatom or ring carbon is the point of attachment of the heterocyclyl substituent to another moiety. Any atom can be optionally substituted, e.g., by one or more substituents. Heterocyclyl groups can include, e.g., tetrahydrofuryl, tetrahydropyranyl, piperidyl (piperidino), piperazinyl, morpholinyl (morpholino), pyrrolinyl, and pyrrolidinyl.

The term “heterocycloalkenyl” refers to partially unsaturated monocyclic, bicyclic, tricyclic, or other polycyclic hydrocarbon groups having one or more (e.g., 1-4) heteroatom ring atoms independently selected from O, N, or S. A ring carbon (e.g., saturated or unsaturated) or heteroatom is the point of attachment of the heterocycloalkenyl substituent. Any atom can be optionally substituted, e.g., by one or more substituents. Heterocycloalkenyl groups can include, e.g., tetrahydropyridyl, dihydropyranyl, 4,5-dihydrooxazolyl, 4,5-dihydro-1H-imidazolyl, 1,2,5,6-tetrahydro-pyrimidinyl, and 5,6-dihydro-2H-[1,3]oxazinyl.

The term “cycloalkyl” refers to a fully saturated monocyclic, bicyclic, tricyclic, or other polycyclic hydrocarbon groups. Any atom can be optionally substituted, e.g., by one or more substituents. A ring carbon serves as the point of attachment of a cycloalkyl group to another moiety. Cycloalkyl moieties can include, e.g., cyclopropyl, cyclobutyl, cyclopentyl, cyclohexyl, cycloheptyl, adamantyl, and norbornyl (bicycle[2.2.1]heptyl).

The term “cycloalkenyl” refers to partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be optionally substituted e.g., by one or more substituents. Cycloalkenyl moieties can include, e.g., cyclohexenyl, cyclohexadienyl, or norbornenyl.

The term “aryl” refers to an aromatic monocyclic or bicyclic hydrocarbon ring system, wherein any ring atom can be optionally substituted, e.g., by one or more substituents. Aryl moieties include phenyl and naphthyl.

The term “heteroaryl” refers to an aromatic monocyclic or bicyclic hydrocarbon groups having one or more (e.g., 1-6) heteroatom ring atoms independently selected from O, N, or S (and mono and dioxides thereof, e.g., N→O⁻, S(O), SO₂). Any atom can be optionally substituted, e.g., by one or more substituents. Heteroaryl groups include pyridyl, thienyl, furyl (furanyl), imidazolyl, isoquinolyl, quinolyl and pyrrolyl.

The descriptor C(O) refers to a carbon atom that is doubly bonded to an oxygen atom.

The term “substituent” refers to a group “substituted” on, e.g., an alkyl, haloalkyl, cycloalkyl, alkenyl, alkynyl, aralkyl, heteroaralkyl, heterocyclyl, heterocycloalkenyl, cycloalkenyl, aryl, or heteroaryl group at any atom of that group. In one aspect, the substituent(s) on a group are independently any one single, or any combination of two or more of the permissible atoms or groups of atoms delineated for that substituent. In another aspect, a substituent may itself be substituted with any one of the above substituents.

In general, when a definition for a particular variable includes both hydrogen and non- hydrogen (halo, alkyl, aryl, etc.) possibilities, the term “substituent(s) other than hydrogen” refers collectively to the non-hydrogen possibilities for that particular variable.

Descriptors such as “C₁-C₆ alkyl which is optionally substituted with from 1-3 R^{j}” (and the like) is intended to include both an unsubstituted C₁-C₆ alkyl group and a C₁-C₆ alkyl group that is substituted with from 1-3 R^{j}. The use of a substituent (radical) prefix names such as alkyl without the modifier “optionally substituted” or “substituted” is understood to mean that the particular substituent is unsubstituted. However, the use of “haloalkyl” without the modifier “optionally substituted” or “substituted” is still understood to mean an alkyl group, in which at least one hydrogen atom is replaced by halo.

In some embodiments, the compounds have agonist activity for genes involved with HDL production and cholesterol efflux (e.g., ABCA1) and antagonist activity for genes involved with triglyceride synthesis (e.g., SREBP-1c).

The details of one or more embodiments of the invention are set forth in the description below. Other features and advantages of the invention will be apparent from the description and from the claims.

### DETAILED DESCRIPTION

This invention relates generally to polar quinazoline-based modulators of Liver X receptors (LXRs) and related methods.

The polar quinazoline-based LXR modulators have the general formula (I):

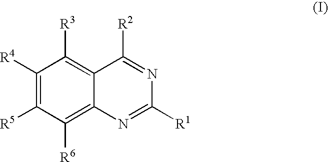

Here and throughout this specification, R¹, R², R³, R⁴, R⁵, R⁶, R²², R²³, R²⁴, R²⁵, R²⁶, R²⁷, R²⁸, R²⁹, W, W¹, W², R^{a}, R^{b}, R^{c}, R^{d}, R^{e}, R^{f}, R^{g}, R^{h}, Rⁱ, R^{j}, R^{m}, Rⁿ, R^{o}, R^{p}, R^{q}, R^{r}, R^{s}, R^{t}, R^{u}, and n can be as defined anywhere herein.

For ease of exposition, it is also understood that where in this specification (including the claims), a group is defined by “as defined anywhere herein” (or the like), the definitions for that particular group include the first occurring and broadest generic definition as well as any sub-generic and specific definitions delineated anywhere in this specification.

Variable R¹

In some embodiments, R¹ can be hydrogen.

In some embodiments, R¹ can be CH₃.

Variable R²

Variables R²², R²³, and R²⁴

In some embodiments, one of R²², R²³, and R²⁴ can be R^{a}, and the other two are hydrogen. In certain embodiments, R²² can be R^{a}, and each of R²³ and R²⁴ can be hydrogen. In these embodiments, R^{a} can be: halo (e.g., chloro or fluoro, e.g., chloro); C₁-C₃ alkyl; or C₁-C₃ haloalkyl (e.g., C₁-C₃ fluoroalkyl, e.g., 1-5 fluorines can be present; or C₁-C₃ perfluoroalkyl). For example, R^{a} can be halo (e.g., fluoro or chloro, e.g., chloro).

In other embodiments, each of R²², R²³, and R²⁴ can be hydrogen. In another embodiment, each of R²², R²³, and R²⁴ can be a substituent other than hydrogen. In still other embodiments, two of R²², R²³, and R²⁴ can be R^{a}, and the other is hydrogen.

Variable W

In some embodiments, W can be a bond, —O—, —NH—, —N(C₁-C₃ alkyl)-, or C₁₋₂ alkylene.

In some embodiments, W can be —O—, —NH—, —N(C₁-C₃ alkyl)-, or C₁₋₂ alkylene.

In certain embodiments, W can be —O—, —NH—, —N(C₁-C₃ alkyl)-, or CH₂.

In certain embodiments, W can be —O—, —NH—, —N(C₁-C₃ alkyl)-.

In certain embodiments, W can be —O—.

In certain embodiments, W can be —NH— or —N(C₁-C₃ alkyl)- (e.g., —N(CH₃)—.

In certain embodiments, W can be CH₂.

In some embodiments, W can be a bond.

Variables R²⁵, R²⁶, R²⁷, R²⁸, and R²⁹

In some embodiments, condition (A) can apply, and one of R²⁵, R²⁶, R²⁷, R²⁸, and R²⁹ (e.g., R²⁶) W¹—S(O)ₙR^{b} or —W¹—S(O)ₙNR^{c}R^{d}; and the others (e.g., R²⁵, R²⁷, R²⁸, and R²⁹) are each, independently, hydrogen or R^{e} (e.g., hydrogen).

In some embodiments, one of R²⁵, R²⁶, R²⁷, R²⁸, and R²⁹ (e.g., R²⁶) can be —W¹—S(O)ₙR^{b}; and the others (e.g., R²⁵, R²⁷, R²⁸, and R²⁹) are each, independently, hydrogen or R^{e} (e.g., hydrogen).

In certain embodiments, W¹ can be a bond.

In certain embodiments, n can be 2.

In certain embodiments, W¹ can be a bond, and n can be 2.

In certain embodiments, R^{b} can be C₁-C₆ (e.g., C₁, C₂-C₆, C₂-C₅, or C₃-C₄) alkyl, which is substituted with from 1-2 R^{o}.

In certain embodiments, R^{b} can be C₁-C₆ (e.g., C₁, C₂-C₆, C₂-C₅, or C₃-C₄) alkyl, which is substituted with 1 R^{o}.

In certain embodiments, R^{b} can be C₁ alkyl, which is substituted with 1 R^{o}.

In certain embodiments, R^{b} can be C₂-C₆ alkyl, which is substituted with 1 R^{o}.

In certain embodiments, R^{b} can be C₂-C₅ alkyl, which is substituted with 1 R^{o}.

In certain embodiments, R^{b} can be C₃-C₄ alkyl, which is substituted with 1 R^{o}.

In these and in the following embodiments related to variable R^{b}, R^{o} can be as defined anywhere herein.

In certain embodiments, R^{o} can be:

(i) —C(O)NR^{t}R^{u}; or

(ii) —C(O)OR^{s}; or

(iii) —NHS(O)ₙR^{f} or —N(C₁-C₃ alkyl)S(O)ₙR^{f}; or

(iv) —S(O)ₙR^{f}.

For example, R^{o} can be —C(O)NR^{t}R^{u} (e.g., —C(O)NH₂, i.e., each of R^{t} and R^{u} is hydrogen).

As another example, R^{o} can be —C(O)OR^{S} (e.g., —C(O)OH, i.e., R^{s} can be hydrogen).

As a further example, R^{o} can be —NHS(O)ₙR^{f} or —N(C₁-C₃ alkyl)S(O)ₙR^{f}. In these embodiments, R^{f} can be C₁-C₃ alkyl (e.g., CH₃). n can be 2. In embodiments, R^{o} can be —NHSO₂CH₃ or —N(CH₃)SO₂CH₃.

As a further example, R^{o} can be —S(O)ₙR^{f}. In these embodiments, R^{f} can be C₁-C₃ alkyl (e.g., CH₃). n can be 2. In embodiments, R^{o} can be —SO₂CH₃.

In certain embodiments, R^{o} can be —CN.

In certain embodiments, R^{o} can be —NR^{m}Rⁿ. In embodiments, each of R^{m} and Rⁿ can be independently, hydrogen or C₁-C₃ alkyl (e.g., CH₃). For example, each of R^{m} and Rⁿ can be hydrogen. As another example, one of R^{m} and Rⁿ can be hydrogen, and the other can be C₁-C₃ alkyl (e.g., CH₃). As a further example, each of R^{m} and Rⁿ can be, independently, C₁-C₃ alkyl.

In certain embodiments (e.g., when R²⁶ is —W¹—S(O)ₙR^{b}), W¹ can be a bond, n can be 2, and R^{b} can be C₁-C₆ (e.g., C₁, C₂-C₆, C₂-C₅, or C₃-C₄) alkyl, which is substituted with from 1 R^{o}.

In embodiments, R^{o} can be:

(i) —C(O)NR^{t}R^{u}; or

(ii) —C(O)OR^{s}; or

(iii) —NHS(O)ₙR^{f} or —N(C₁-C₃ alkyl)S(O)ₙR^{f}; or

(iv) —S (O)ₙR^{f}.

In embodiments, R^{o} can be —CN.

In embodiments, R^{o} can be —NR^{m}Rⁿ, in which R^{m} and Rⁿ can be independently as defined anywhere herein.

In certain embodiments (e.g., when R²⁶ is —W¹—S(O)ₙR^{b}), W¹ can be a bond, n can be 2, and R^{b} can be C₁ alkyl, which is substituted with from 1 R^{o}.

In embodiments, R^{o} can be:

(i) —C(O)NR^{t}R^{u}; or

(ii) —C(O)OR^{s}; or

(iii) —NHS(O)ₙR^{f} or —N(C₁-C₃ alkyl)S(O)ₙR^{f}; or

(iv) —S(O)ₙR^{f}.

For example, R^{o} can be —C(O)NR^{t}R^{u} (e.g., —C(O)NH₂, i.e., each of R^{t} and R^{u} is hydrogen).

As another example, R^{o} can be —C(O)OR^{s} (e.g., —C(O)OH, i.e., R^{s} can be hydrogen).

In other embodiments, R^{o} can be —CN.

In certain embodiments (e.g., when R²⁶ is —W¹—S(O)ₙR^{b}), W¹ can be a bond, n can be 2, and R^{b} can be C₂-C₅ alkyl, which is substituted with from 1 R^{o}.

In embodiments, R^{o} can be:

(i) —C(O)NR^{t}R^{u}; or

(ii) —C(O)OR^{s}; or

(iii) —NHS(O)ₙR^{f} or —N(C₁-C₃ alkyl)S(O)ₙR^{f}; or

(iv) —S(O)ₙR^{f}.

For example, R^{o} can be —C(O)NR^{t}R^{u} (e.g., —C(O)NH₂, i.e., each of R^{t} and R^{u} is hydrogen).

As another example, R^{o} can be —C(O)OR^{s} (e.g., —C(O)OH, i.e., R^{s} can be hydrogen).

As a further example, R^{o} can be —NHS(O)ₙR^{f} or —N(C₁-C₃ alkyl)S(O)ₙR^{f}. In these embodiments, R^{f} can be C₁-C₃ alkyl (e.g., CH₃). n can be 2. In embodiments, R^{o} can be —NHSO₂CH₃ or —N(CH₃)SO₂CH₃.

As a further example, R^{o} can be —S(O)ₙR^{f}. In these embodiments, R^{f} can be C₁-C₃ alkyl (e.g., CH₃). n can be 2. In embodiments, R^{o} can be —SO₂CH₃.

In other embodiments, R^{o} can be —CN.

In still other embodiments, R^{o} can be —NR^{m}Rⁿ (e.g., NH₂).

In certain embodiments (e.g., when R²⁶ is —W¹—S(O)ₙR^{b}), W¹ can be a bond, n can be 2, and R^{b} can be C₃-C₄ alkyl, which is substituted with from 1 R^{o}.

In embodiments, R^{o} can be:

(i) —C(O)NR^{t}R^{u}; or

(ii) —C(O)OR^{s}; or

(iii) —NHS(O)ₙR^{f} or —N(C₁-C₃ alkyl)S(O)ₙR^{f}; or

(iv) —S(O)ₙR^{f}.

For example, R^{o} can be —C(O)NR^{t}R^{u} (e.g., —C(O)NH₂, i.e., each of R^{t} and R^{u} is hydrogen).

As another example, R^{o} can be —C(O)OR^{s} (e.g., —C(O)OH, i.e., R^{s} can be hydrogen).

As a further example, R^{o} can be —NHS(O)ₙR^{f} or —N(C₁-C₃ alkyl)S(O)ₙR^{f}. In these embodiments, R^{f} can be C₁-C₃ alkyl (e.g., CH₃). n can be 2. In embodiments, R^{o} can be —NHSO₂CH₃ or —N(CH₃)SO₂CH₃.

As a further example, R^{o} can be —S(O)ₙR^{f}. In these embodiments, R^{f} can be C₁-C₃ alkyl (e.g., CH₃). n can be 2. In embodiments, R^{o} can be —SO₂CH₃.

In other embodiments, R^{o} can be —CN.

In still other embodiments, R^{o} can be —NR^{m}Rⁿ (e.g., NH₂).

In certain embodiments, R²⁶ can be —W¹—S(O)ₙR^{b}, in which W¹, n, R^{b}, and R^{o} can be as defined anywhere herein. In these embodiments, each of R²⁵, R²⁷, R²⁸, and R²⁹ can be hydrogen.

In some embodiments, one of R²⁵, R²⁶, R²⁷, R²⁸, and R²⁹ (e.g., R²⁶) can be —W¹—S(O)ₙNR^{c}R^{d}; and the others (e.g., R²⁵, R²⁷, R²⁸, and R²⁹) are each, independently, hydrogen or R^{e} (e.g., hydrogen).

In certain embodiments, W¹ can be a bond.

In certain embodiments, n can be 2.

In certain embodiments, W¹ can be a bond, and n can be 2.

In certain embodiments, each of R^{c} and R^{d} can be independently, hydrogen or C₁-C₃ alkyl (e.g., CH₃). For example, each of R^{c} and R^{d} can be hydrogen. As another example, one of R^{c} and R^{d} can be hydrogen, and the other can be C₁-C₃ alkyl (e.g., CH₃). As a further example, each of R^{c} and R^{d} can be, independently, a substituent other than hydrogen, e.g., C₁-C₃ alkyl; or C₇-C₁₁ aralkyl, which is optionally substituted with from 1-3 R^{q}.

In certain embodiments (e.g., when R²⁶ is —W¹—S(O)ₙNR^{c}R^{d}), W¹ can be a bond, and n can be 2, and each of R^{c} and R^{d} can be independently, hydrogen or C₁-C₃ alkyl (e.g., CH₃). For example, each of R^{c} and R^{d} can be hydrogen. As another example, one of R^{c} and R^{d} can be hydrogen, and the other can be C₁-C₃ alkyl (e.g., CH₃).

In certain embodiments (e.g., when R²⁶ is —W¹—S(O)ₙNR^{c}R^{d}), W¹ can be a bond, and n can be 2, and each of R^{c} and R^{d} can be, independently, a substituent other than hydrogen, e.g., C₁-C₃ alkyl or C₇-C₁₁ aralkyl, which is optionally substituted with from 1-3 R^{q}.

In certain embodiments, R²⁶ can be —W¹—S(O)ₙNR^{c}R^{d}, in which W¹, n, R^{c}, and R^{d} can be as defined anywhere herein. In these embodiments, each of R²⁵, R²⁷, R²⁸, and R²⁹ can be hydrogen.

In some embodiments, condition (B) can apply, and

(a) one of R²⁵, R²⁶, R²⁷, R²⁸, and R²⁹ (e.g., R²⁶) can be —W¹—S(O)ₙR^{f}; and

(b) one of R²⁵, R²⁶, R²⁷, R²⁸, and R²⁹ (e.g., R²⁷) can be —W¹—C(O)OR^{g}, —W¹—C(O)NR^{h}R^{l}; or —W²—CN; and

(c) the others are each, independently, hydrogen or R^{e} (e.g., hydrogen).

In certain embodiments:

(a) one of R²⁵, R²⁶, R²⁷, R²⁸, and R²⁹ (e.g., R²⁶) can be —W¹—S(O)ₙR^{f}; and

(b) one of R²⁵, R²⁶, R²⁷, R²⁸, and R²⁹ (e.g., R²⁷) can be —W¹—C(O)OR^{g} or —W¹—C(O)NR^{h}Rⁱ; and

(c) the others are each, independently, hydrogen or R^{e} (e.g., hydrogen).

In certain embodiments:

(a) one of R²⁵, R²⁶, R²⁷, R²⁸, and R²⁹ (e.g., R²⁶) can be —W¹—S(O)ₙR^{f}; and

(b) one of R²⁵, R²⁶, R²⁷, R²⁸, and R²⁹ (e.g., R²⁷) can be —W²—CN; and

(c) the others are each, independently, hydrogen or R^{e} (e.g., hydrogen).

In these embodiments, one or more of the following can apply. W¹ at each occurrence can be a bond. W² can be a bond. R^{f} can be C₁-C₃ alkyl (e.g., CH₃). Each of R^{h} and Rⁱ can be hydrogen. R^{g} can be hydrogen.

For example:

(a) one of R²⁵, R²⁶, R²⁷, R²⁸, and R²⁹ (e.g., R²⁶) can be —SO₂CH₃; and

(b) one of R²⁵, R²⁶, R²⁷, R²⁸, and R²⁹ (e.g., R²⁷) can be —C(O)OH or —C(O)NH₂; and

(c) the others are each, independently, hydrogen or R^{e} (e.g., hydrogen).

In certain embodiments:

(a) R²⁶ can be —W¹—S(O)ₙR^{f}; and

(b) R²⁷ can be —W¹—C(O)OR^{g} or —W¹—C(O)NR^{h}Rⁱ; and

(c) each of R²⁵, R²⁸, and R²⁹ is, independently, hydrogen or R^{e} (e.g., hydrogen).

In certain embodiments:

(a) R²⁶ can be —W¹—S(O)ₙR^{f}; and

(b) R²⁷ can be —W²—CN; and

(c) each of R²⁵, R²⁸, and R²⁹ is, independently, hydrogen or R^{e} (e.g., hydrogen).

In these embodiments, one or more of the following can apply. W¹ at each occurrence can be a bond. W² can be a bond. R^{f} can be C₁-C₃ alkyl (e.g., CH₃). Each of R^{h} and Rⁱ can be hydrogen. Rg can be hydrogen. Each of R²⁵, R²⁸, and R²⁹ is hydrogen.

For example, R²⁶ can be —SO₂R^{f} (e.g., —SO₂CH₃)

For example, R²⁷ can be —C(O)OR^{g} or —C(O)NR^{h}Rⁱ (e.g., —C(O)OH or —C(O)NH₂).

For example, R²⁷ can be —CN.

For example, R²⁶ can be —SO₂R^{f}, and R²⁷ can be —C(O)OR^{g} or —C(O)NR^{h}Rⁱ (e.g., R²⁶ can be —SO₂CH₃, and R²⁷ can be —C(O)OH or —C(O)NH₂).

For example, R²⁶ can be —SO₂R^{f} (e.g., —SO₂CH₃) and R²⁷ can be —CN.

Variables R³, R⁴, and R⁵

In some embodiments, each of R³, R⁴ and R⁵ can be, independently, hydrogen or halo (e.g., fluoro).

In some embodiments, each of R³, R⁴ and R⁵ can be hydrogen.

In some embodiments, each of R³, R⁴ and R⁵ can be a substituent other than hydrogen (e.g., halo, e.g., fluoro).

Variable R⁶

In some embodiments, R⁶ can be C₁-C₄ haloalkyl (e.g., C₁-C₄ perfluoroalkyl, e.g., CF₃).

In some embodiments, R⁶ can be halo (e.g., chloro).

In general, the compounds of formula (I) have a Topological Polar Surface Area (“TPSA”) of greater than about 80 Å². TPSA refers to the sum of surfaces of polar atoms (usually oxygens, nitrogens and attached hydrogens) in a molecule as determined by summation of tabulated surface contributions of polar fragments (atoms regarding also their environment). Methods for calculating TPSA are known in the art, see, e.g., Ertl, P., Rohde, B., Selzer, P. “Fast calculation of molecular polar surface area as a sum of fragment based contributions and its application to the prediction of drug transport properties.” J. Med. Chem. 2000, 43: 3714-3717. While not wishing to be bound by theory, it is believed that compounds having a (TPSA) of greater than about 80 Å² have a reduced likelihood of penetrating the brain (see, e.g., Gleeson, J. Med. Chem. 2008, 51, 817), which can potentially reduce the CNS-related side effects.

In some embodiments, the compounds of formula (I) can have a TPSA of from about 100 Å² to about 120 Å².

In some embodiments, the compounds of formula (I) can have TPSA of from about 100 Å² to about 109 Å².

In some embodiments, the compounds of formula (I) can have TPSA of from about 110 Å² to about 120 Å².

In some embodiments, the compounds of formula (I) can have TPSA of from about 90 Å² to about 99 Å².

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species.

The compounds described herein can be synthesized according to methods described herein (or variations thereof) and/or conventional, organic chemical synthesis methods from commercially available starting materials and reagents or from starting materials and reagents that can be prepared according to conventional organic chemical synthesis methods. The compounds described herein can be separated from a reaction mixture and further purified by a method such as column chromatography, high-performance liquid chromatography (HPLC), or recrystallization. As can be appreciated by the skilled artisan, further methods of synthesizing the compounds of the formulae herein will be evident to those skilled in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. C. Larock, Comprehensive Organic Transformations, 2d.ed., Wiley-VCH Publishers (1999); P. G. M. Wuts and T. W. Greene, Protective Groups in Organic Synthesis, 4th Ed., John Wiley and Sons (2007); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention can be readily prepared according to the following schemes from commercially available starting materials or starting materials which can be prepared using literature procedures. The schemes show the preparation of representative compounds of this invention. It is also possible to make use of variants of these process steps, which in themselves are known to and well within the preparatory skill of the skilled artisan.

In some embodiments, compounds of formula (I) can be prepared according to Scheme 1.

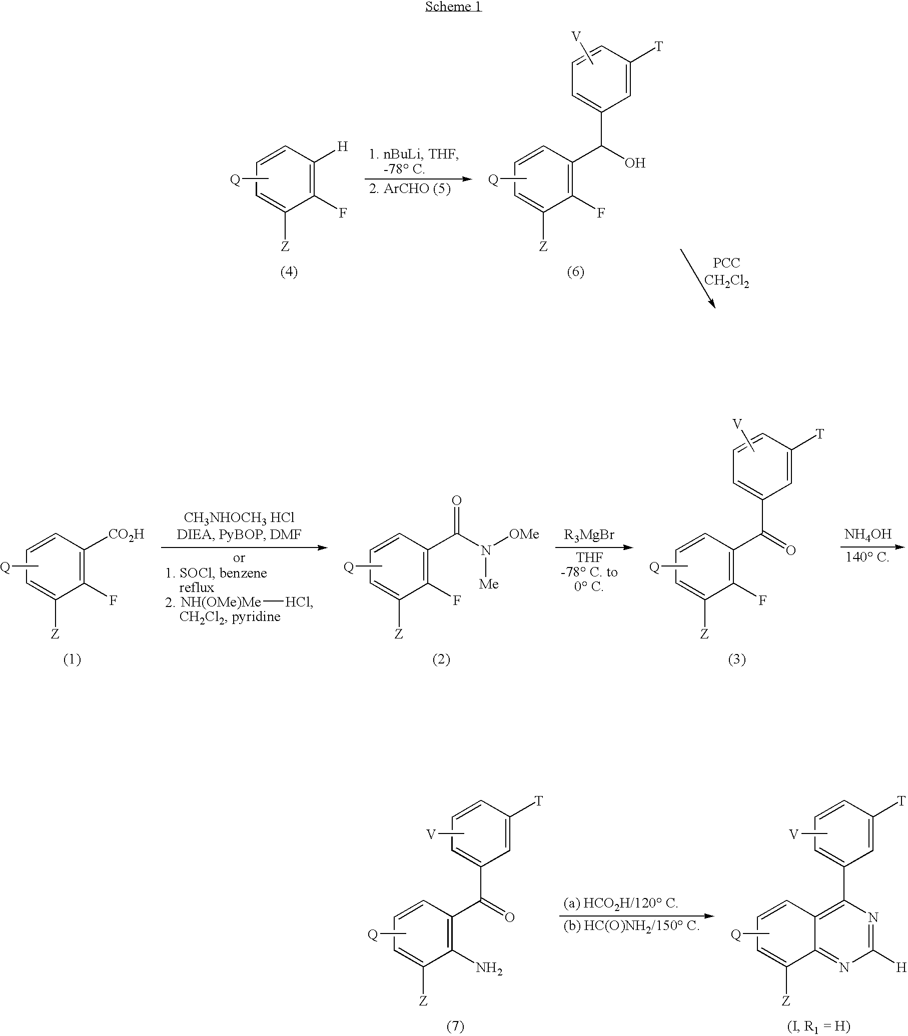

The term “Q” in Scheme 1 corresponds to R³, R⁴, and R⁵ in formula (I) or is a substituent precursor thereto. The term “Z” in Scheme 1 corresponds to R⁶ in formula (I) or is a substituent precursor thereto. The term “V” in Scheme 1 corresponds to R²², R²³, and R²⁴ in formula (I) (and as defined anywhere herein) or is a substituent precursor thereto. The term “T” in Scheme 1 corresponds to the following formula

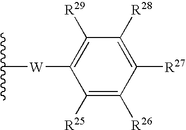

in formula (I) or is a substituent precursor thereto.

According to Scheme 1, the compounds of formula (I) can be prepared by converting compounds of formula (1) to the N-methyl, N-methoxy amide (“Weinreb amide”) of formula (2) under conventional amidation conditions. Reaction of the amide (2) with a lithio or Grignard reagent (ArLi or ArMgBr) at low temperature can provide the ketone of formula (3). Alternatively, the compound of formula (4) can be lithiated alpha to fluorine and then treated with an appropriately substituted aldehyde of formula (5). The resulting alcohol (6) can be converted to the ketone (3) under conventional oxidation conditions. Conversion of ketone (3) into the aniline of formula (7) can be accomplished with ammonium hydroxide at elevated temperature or by using a protected amine followed by deprotection. Substituted anilines of formula (7) can then undergo cyclization in formic acid with formamide at elevated temperature to provide compounds of formula (I).

In some embodiments, compounds of formula (I) can be prepared according to Scheme 2.

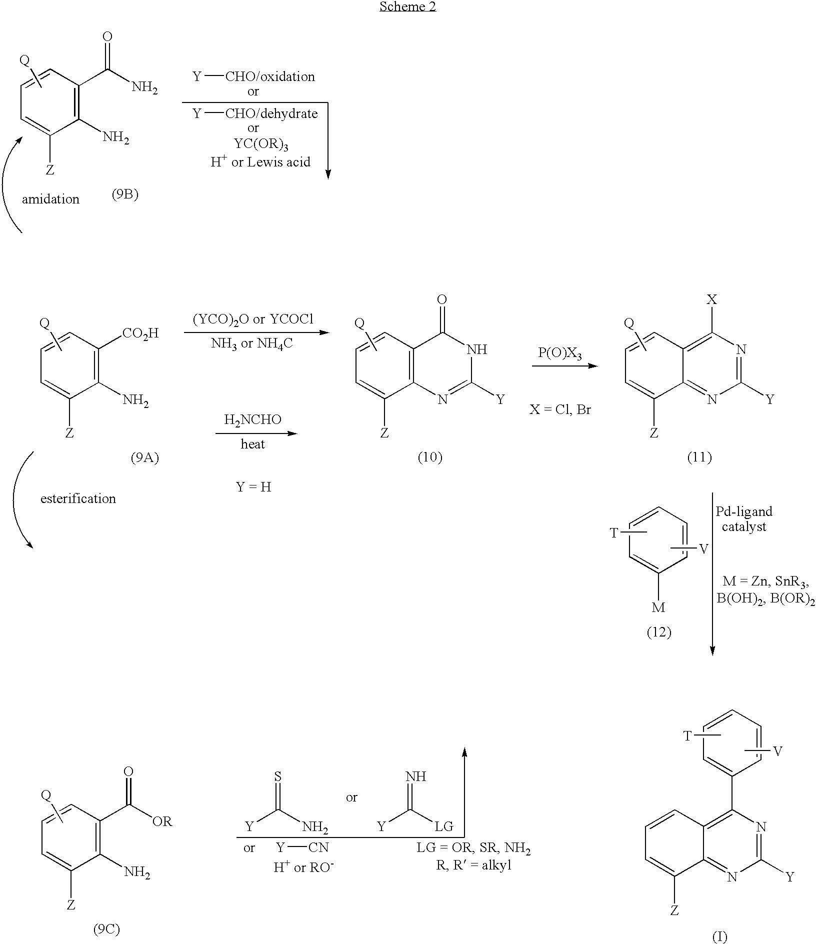

The meanings of “Q,” “Z,” “V,” and “T” in Scheme 2 are the same as indicated above for Scheme 1. The term “Y” in Scheme 2 corresponds to R¹ in formula (I) or is a substituent precursor thereto.

Several methodologies are available to convert appropriately substituted anthranilic acid derivatives, such as 9A, 9B, 9C, into the quinazolone derivatives (10) (see Scheme 2). Treatment of (10) with certain phospho-halogen reagents such as phosphorous oxychloride, phosphorous oxybromide, or other reagents known to those skilled in the art lead to 4-halo-quinazoline compounds (11). Compound (11) may be reacted with an appropriately substituted arylboronic acid, arylzincate, or arystannane of formula (12) using palladium-(tetrakistriphenylphosphine) or other liganded palladium catalysts known to those skilled in the art, to yield a compound of formula (I).

In some embodiments, compounds of formula (I) can be prepared according to Scheme 3.

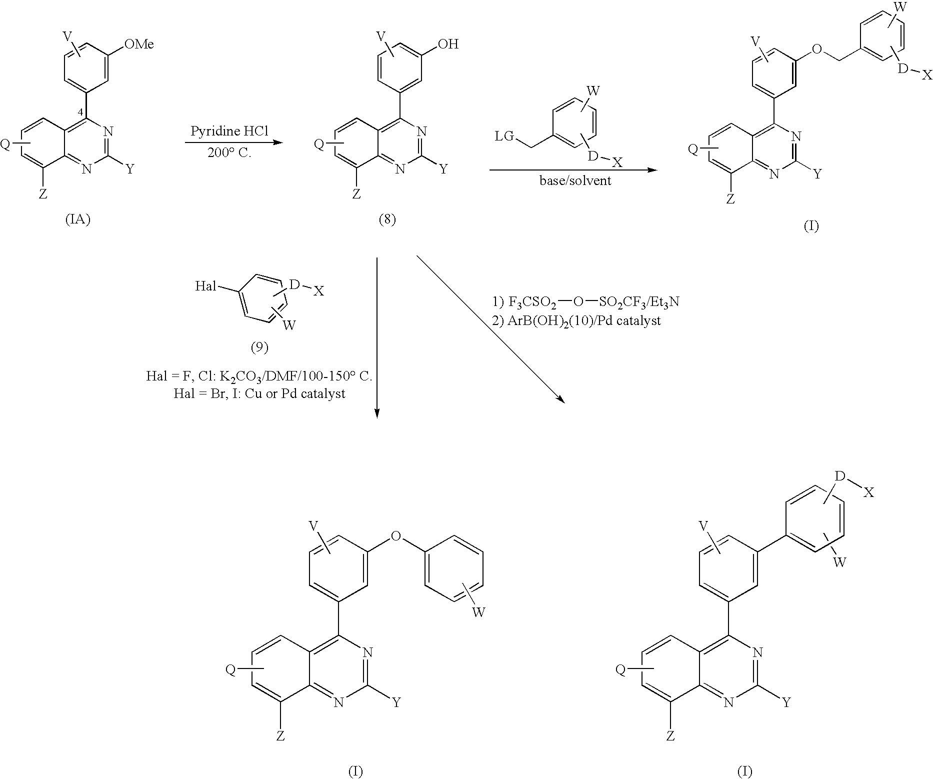

The meanings of “Q,” “Z,” “V,” and “Y” in Scheme 3 are the same as indicated above for Schemes 1 and 2. The terms “W” and “D-X” in Scheme 3 correspond to R²⁵, R²⁶, R²⁷, R²⁸, and R²⁹ in formula (I) as defined anywhere herein or are substituent precursors thereto. The term “LG” in Scheme 3 represents a leaving group.

According to Scheme 3, certain compounds of formula (I) in which T=OMe, prepared by Scheme 1 or Scheme 2, can be converted by demethylation under standard conditions including pyridine hydrochloride at elevated temperature or by treatment with HI in acetic acid to provide phenols of formula (8). Alkylation of the phenols of formula (8) with an alkylating agent RX′ using potassium carbonate, sodium carbonate, or cesium carbonate as the base can provide an alkylated compound of formula (I). If the R group of the compound of formula (I) contains a carboxylic acid ester moiety, this moiety can be transformed to the carboxylic acid upon treatment with aqueous lithium hydroxide, sodium hydroxide, or potassium hydroxide in a suitable organic solvent. If the R group of the compound of formula (I) contains a CH₂X′ group where X′ is a halogen, for example Br or Cl, then this group can be transformed to CH₂CN upon treatment with sodium cyanide in a suitable organic solvent. Alternatively, compounds of formula (I) in which T=OH can be treated with a halogenated aromatic ring to provide a biarylether of formula (I). If the halogen is a fluorine or chlorine atom, the formation of the biarylether of formula (I) can be accomplished by treatment with a base such as potassium carbonate, typically in a polar solvent such as dimethylformamide or dimethylsulfoxide, at elevated temperatures, typically 100° C. to 150° C. for several hours. Alternatively, where the halogen is bromine or iodine, the formation of the biarylether (I) can be accomplished with a coupling reaction using a metal catalyst such as a copper salt or a palladium salt in the presence of a base and a solvent such as dioxane at elevated temperatures. Where a compound of formula (I) in which a direct bond to the 4-phenyl ring is desired, the phenol of compounds of formula (I) in which T=OH can be converted into a triflate using triflic anhydride and a tertiary amine such as triethylamine, or to a bromide (T=Br). The resulting triflate or bromine of formula (9) can be coupled to an arylboronic acid of formula (10) under catalysis with a palladium catalyst, a reaction known in the art as a Suzuki reaction.

In some embodiments, compounds of formula (I) can be prepared according to Scheme 4.

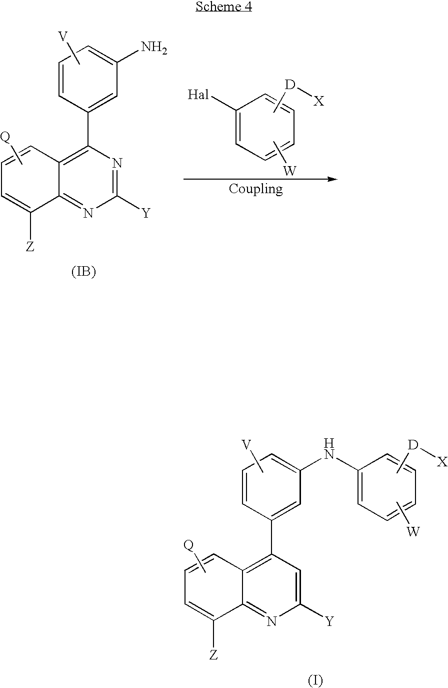

The meanings of “Q,” “Z,” “V,” “Y,” “W,” and “D-X” in Scheme 4 are the same as indicated above for Schemes 1, 2, and 3.

According to Scheme 4, certain compounds of formula (I) prepared by Scheme 1 or Scheme 2, contain a free NH₂ moiety on the phenyl ring that is attached to the 4-position of the quinazoline ring system. Treatment of the free NH₂ compound of formula (I) with an aryl halide (or aryltriflate or arylboronic acid) of formula Hal-Ar-D-X, optionally substituted with a group W, can provide the corresponding biarylamine of formula (I).

In certain embodiments, an aniline or phenol can be arylated, as shown in Scheme 5, with a boronic acid in the presence of Cu(OAc)₂, an amine base such as pyridine or triethylamine, with or without additives such as myristic acid or molecular sieves, in an inert solvent such as dichloromethane at room temperature or an elevated temperature.

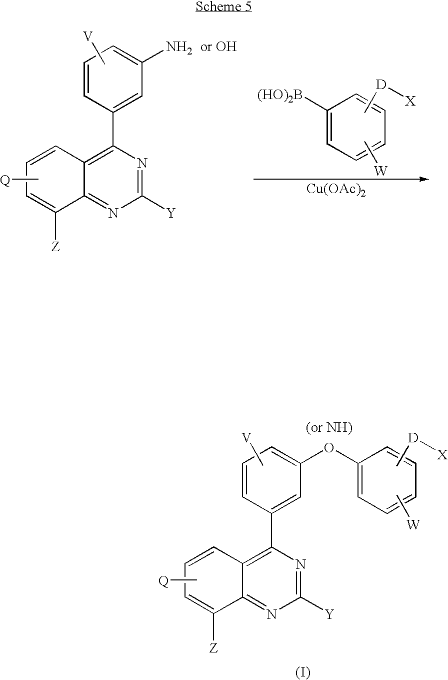

The meanings of “Q,” “Z,” “V,” “Y,” “W,” and “D-X” in Scheme 5 are the same as indicated above for Schemes 1, 2, and 3.

In some embodiments, compounds of formula (I) can be prepared according to Scheme 6.

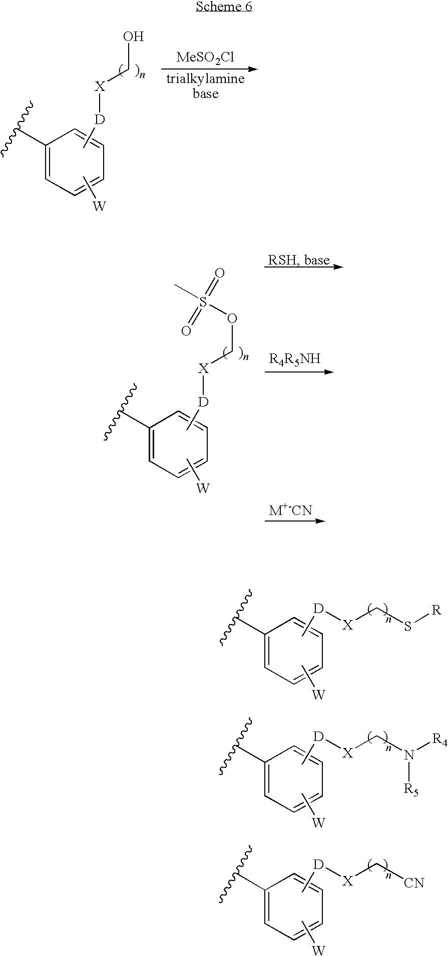

The meangings of “W” and the “D-X” containing groups in Scheme 6 are the same as indicated above for Schemes 1, 2, and 3.

According to Scheme 6, modification of the W or R group can be accomplished as follows. For example, alkanols can be transformed into other fuctionalities by conversion of the hydroxyl group to a leaving group, such as a methanesulfonate, followed by displacement with a nucleophile such as a metal cyanide, a thiol, an amine, etc. When W, X, and/or R is a sulfide, it can be oxidized to a sulfoxide or sulfone; When W, X, and/or R is an amine, it can be acylated or sulfonylated; and When W, X, and/or R is a nitrile, it can be hydrolyzed to an amide or an acid. Such transformations are known in the art.

The compounds of this invention may contain one or more asymmetric centers and thus occur as racemates and racemic mixtures, enantiomerically enriched mixtures, single enantiomers, individual diastereomers and diastereomeric mixtures. All such isomeric forms of these compounds are expressly included in the present invention. The compounds of this invention may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include C₁₋₆ alkyl esters of carboxylic acid groups, which, upon administration to a subject, are capable of providing active compounds.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The term “pharmaceutically acceptable carrier or adjuvant” refers to a carrier or adjuvant that may be administered to a subject (e.g., a patient), together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

In general, the compounds described herein can be used for, treating (e.g., controlling, relieving, ameliorating, alleviating, slowing the progression of, delaying the onset of, or reducing the risk of developing) or preventing one or more diseases, disorders, conditions or symptoms mediated by LXRs (e.g., cardiovascular diseases (e.g., acute coronary syndrome, restenosis, or coronary artery disease), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

A disorder or physiological condition that is mediated by LXR refers to a disorder or condition wherein LXR can trigger the onset of the condition, or where inhibition of a particular LXR can affect signaling in such a way so as to treat, control, ameliorate, alleviate, prevent, delay the onset of, slow the progression of, or reduce the risk of developing the disorder or condition. Examples of such disorders include, but are not limited to cardiovascular diseases (e.g., acute coronary syndrome, restenosis, or coronary artery disease), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

While not wishing to be bound by theory, it is believed that LXR modulators that activate cholesterol efflux (e.g., upregulate ABCA1), but do not substantially increase SREBP-1c expression and triglyceride synthesis in liver, can both reduce atherosclerotic risk and minimize the likelihood of concommitantly increasing serum and hepatic triglyceride levels. Candidate compounds having differential activity for regulating ABCA1 (ABCG1) vs. SREBP-1c can be can be evaluated using conventional pharmacological test procedures, which measure the affinity of a candidate compound to bind to LXR and to upregulate the gene ABCA1.

In some embodiments, LXR ligands can be identified initially in cell-free LXR beta and LXR alpha competition binding assays. LXR ligands can be further characterized by gene expression profiling for tissue selective gene regulation.

In some embodiments, the compounds described herein have agonist activity for ABCA1 transactivation but do not substantially affect (e.g., inhibit) SREBP-1c gene expression in differentiated THP-1 macrophages. Gene expression analysis in an antagonist mode can be used to further delineate differential regulation of ABCA1 and SREBP-1c gene expression. In certain embodiments, the compounds described herein preferentially antagonize SREBP-1c activation (a marker for genes involved in cholesterol and fatty acid homeostasis) but do not substantially affect (e.g., have relatively minimal or additive effects) on ABCA1 gene expression or genes known to enhance HDL biogenesis (based on a competition assay with known potent synthetic LXR agonists). Cell type or tissue specificity may be further evaluated in additional cell lines, intestinal, CaCo2 or liver, HepG2 and Huh-7 cells where ABCA1 activity is believed to influence net cholesterol absorption and reverse cholesterol transport. The test procedures performed, and results obtained therefrom are described in the Examples section.

In some embodiments, the compounds described herein have agonist activity for ABCA1 and antagonist activity for SREBP-1c (e.g., as determined by gene specific modulation in cell based assays). In certain embodiments, the compounds described herein (in the agonist mode) have at least about 20% efficacy for ABCA1 activation by LXR and do not substantially agonize SREBP-1c (at most about 25% efficacy relative to a reference compound N-(2,2,2-trifluoro-ethyl)-N-[4-(2,2,2-trifluoro-1-hydroxy-1-trifluoromethyl-ethyl)-phenyl]-benzenesulfonamide (Schultz, Joshua R., Genes & Development (2000), 14(22), 2831-2838)). In certain embodiments, the compounds described herein (in the antagonist mode) do not substantially antagonize ABCA1 gene expression. While not wishing to be bound by theory, it is believed that there may be an additive effect on ABCA1 gene expression relative to the reference compound at their EC₅₀ concentration. In certain embodiments, the compounds described herein (in the antagonist mode) inhibited agonist-mediated SREBP-1c gene expression in a dose dependent fashion.

In some embodiments, to study the effect of the compounds of formula (I) on skin aging, for example, in a clinical trial, cells can be isolated and RNA prepared and analyzed for the levels of expression of TIMP1, ABCA12, decorin, TNFα, MMP1, MMP3, and/or IL-8. The levels of gene expression (i.e., a gene expression pattern) can be quantified, for example, by Northern blot analysis or RT-PCR, by measuring the amount of protein produced, or by measuring the levels of activity of

TIMP1, ABCA12, decorin, TNFα, MMP1, MMP3, and/or IL-8, all by methods known to those of ordinary skill in the art. In this way, the gene expression pattern can serve as a marker, indicative of the physiological response of the cells to the compounds of formula (I). Accordingly, this response state may be determined before, and at various points during, treatment of the individual with the compounds of formula (I).

In one embodiment, expression levels of cytokines and metalloproteases described herein can be used to facilitate design and/or identification of compounds that treat skin aging through an LXR-based mechanism. Accordingly, the invention provides methods (also referred to herein as “screening assays”) for identifying modulators, i.e., LXR modulators, that have a stimulatory or inhibitory effect on, for example, TIMP1, ABCA12, decorin, TNFα, MMP1, MMP3, and/or IL-8 expression.

An exemplary screening assay is a cell-based assay in which a cell that expresses LXR is contacted with a test compound, and the ability of the test compound to modulate TIMP1, ABCA12, decorin, TNFα, MMP1, MMP3, and/or IL-8 expression through an LXR-based mechanism. Determining the ability of the test compound to modulate TIMP1, ABCA12, decorin, TNFα, MMP1, MMP3, and/or IL-8 expression can be accomplished by monitoring, for example, DNA, mRNA, or protein levels, or by measuring the levels of activity of TIMP1, ABCA12, decorin, TNFα, MMP1, MMP3, and/or IL-8, all by methods known to those of ordinary skill in the art. The cell, for example, can be of mammalian origin, e.g., human.

In some embodiments, to study the effect of the compounds of formula (I) on osteoarthritis, for example, in a clinical trial, cells can be isolated and RNA prepared and analyzed for the levels of expression of ApoD and other genes implicated in osteoarthritis (for example, TNFα). The levels of gene expression (i.e., a gene expression pattern) can be quantified by Northern blot analysis or RT-PCR, by measuring the amount of protein produced, or by measuring the levels of activity of ApoD or other genes, all by methods known to those of ordinary skill in the art. In this way, the gene expression pattern can serve as a marker, indicative of the physiological response of the cells to the LXR modulator. Accordingly, this response state may be determined before, and at various points during, treatment of the individual with the LXR modulator.

An exemplary screening assay is a cell-based assay in which a cell that expresses LXR is contacted with a test compound, and the ability of the test compound to modulate ApoD expression and/or aggrecanase activity and/or cytokine elaboration through an LXR-based mechanism. Determining the ability of the test compound to modulate ApoD expression and/or aggrecanase activity and/or cytokine elaboration can be accomplished by monitoring, for example, DNA, mRNA, or protein levels, or by measuring the levels of activity of ApoD, aggrecanase, and/or TNFα, all by methods known to those of ordinary skill in the art. The cell, for example, can be of mammalian origin, e.g., human.

In some embodiments, the compounds described herein can be coadministered with one or more other threapeutic agents. In certain embodiments, the additional agents may be administered separately, as part of a multiple dose regimen, from the compounds of this invention (e.g., sequentially, e.g., on different overlapping schedules with the administration of one or more compounds of formula (I) (including any subgenera or specific compounds thereof)). In other embodiments, these agents may be part of a single dosage form, mixed together with the compounds of this invention in a single composition. In still another embodiment, these agents can be given as a separate dose that is administered at about the same time that one or more compounds of formula (I) (including any subgenera or specific compounds thereof) are administered (e.g., simultaneously with the administration of one or more compounds of formula (I) (including any subgenera or specific compounds thereof)). When the compositions of this invention include a combination of a compound of the formulae described herein and one or more additional therapeutic or prophylactic agents, both the compound and the additional agent can be present at dosage levels of between about 1 to 100%, and more preferably between about 5 to 95% of the dosage normally administered in a monotherapy regimen.

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/kg to about 1000 mg/kg, (e.g., from about 0.01 to about 100 mg/kg, from about 0.1 to about 100 mg/kg, from about 1 to about 100 mg/kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient.

See, e.g., Scientific Tables, Geigy Pharmaceuticals, Ardsley, New York, 537 (1970). In certain embodiments, the compositions are administered by oral administration or administration by injection. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of this invention is useful when the desired treatment involves areas or organs readily accessible by topical application. For application topically to the skin, the composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of this invention may also be topically applied to the lower intestinal tract by rectal suppository formulation or in a suitable enema formulation.

In some embodiments, topical administration of the compounds and compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Remington's Pharmaceutical Sciences, 21st Edition (2005) published by Mack Publishing Company, which is incorporated herein by reference in its entirety.

Topically-transdermal patches are also included in this invention. Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

The compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

The invention will be further described in the following examples. It should be understood that these examples are for illustrative purposes only and are not to be construed as limiting this invention in any manner.

### Examples

The following describes the preparation of representative compounds of this invention. Compounds described as homogeneous are determined to be of 90% or greater purity (exclusive of enantiomers) by analytical reverse phase chromatographic analysis with 254 nM UV detection. Melting points are reported as uncorrected in degrees centigrade. Mass spectral data is reported as the mass-to-charge ratio, m/z; and for high resolution mass spectral data, the calculated and experimentally found masses, [M+H]⁺ or [M−H]⁻, for the neutral formulae M are reported. All reactions are stirred and run under a nitrogen atmosphere unless otherwise noted. Eluents for chromatography are indicated by E for ethyl acetate and H for hexanes. Thus, for example, the expression “30:70 E:H” refers to a mixture of 30% ethyl acetate and 70% hexanes by volume.

### Example 1

### 4-[4-chloro-3-(8-chloroquinazolin-4-yl)phenoxy]-2-(methylsulfonyl)benzonitrile

### Step 1: 8-chloroquinazolin-4(3H)-one

A slurry of 2-amino-3-chlorobenzoic acid (3.26 g, 8.8 mmol) was heated in formamide (5 mL) at 135° C. for 90 min, then at 175° C. for 90 min. The mixture was cooled to rt and poured into water (150 mL). The solid was collected and washed with 0.1N NH₄OH (100 mL). The off-white solid was used without further purification. MS (ESI) m/z 179.2; HRMS: calcd for C₈H₅ClN₂O+H⁺, 181.01632; found (ESI, [M+H]⁺ Obs'd), 181.0164.

### Step 2: 4,8-dichloroquinazoline

A suspension of 8-chloroquinazolin-4(3H)-one (2.74 g, 15.2 mmol) and DMF (200 μL) was heated in thionyl chloride (80 mL) at 72° C. for 8 h, with venting, during which the solution became homogeneous. The solution was slowly added into vigourously stirred ice/water (gas evolution!) and the resulting solid was collected. The solid was washed with water and dried to yield an off-white solid that was used without further purification. MS (ESI) m/z 181.0

### Step 3: 8-chloro-4-(2-chloro-5-methoxyphenyl)quinazoline

A stream of nitrogen gas was bubbled through a mixture of 4,8-dichloroquinazoline (1.75 g, 8.8 mmol), 2-chloro-5-methoxyphenylboronic acid (1.97 g, 10.6 mmol), 2M aqueous Na₂CO₃ (11 mL, 22 mmol) in dimethoxyethane (15 mL) and water (4 mL) for 10 min. Tetrakis-triphenylphosphine palladium (521 mg, 0.44 mmol) was added and the mixture was stirred at 75° C. for 6 h. The suspension was cooled and partitioned between EtOAc (60 mL) and water (30 mL). The layers were separated and the organic layer was further washed with aqueous NaHCO₃ (10 mL), water (10 mL), and brine (20 mL). The organic layer was dried with Na₂SO₄ and concentrated in vacuo. The residue was purified by silica gel chromatography eluting with a gradient of 0:100 to 20:80 E:H to afford white foamy solid. MS (ESI) m/z 305.0; HRMS: calcd for C₁₅H₁₀Cl₂N₂O+H⁺, 305.02429; found (ESI, [M+H]⁺ Obs'd), 305.0247.

### Step 4: 4-chloro-3-(8-chloroquinazolin-4-yl)phenol

A mixture of 8-chloro-4-(2-chloro-5-methoxyphenyl)quinazoline (1.8 g, 5.9 mmol) and solid pyridine hydrochloride (20 g, 173 mmol) was heated to 200° C., during which it became a homogenous solution. After 1.5 h, the mixture was partitioned between (60 mL)/EtOAc (120 mL) and the layers were separated. The organic layer was further washed with 5% citric acid (2×30 mL), aqueous NaHCO₃ (20 mL), and brine (20 mL). The organic layer was dried with Na₂SO₄ and concentrated in vacuo. The crude solid was recystallized from chloroform/hexanes to give a pink solid. MS (ESI) m/z 291.0; HRMS: calcd for C₁₄H₈Cl₂N₂O+H⁺, 291.00864; found (ESI, [M+H]⁺ Obs'd), 291.0089.

### Step 5: 4-[4-chloro-3-(8-chloroquinazolin-4-yl)phenoxyl-2-(methylsulfonyl)benzonitrile

A stirred mixture of 4-chloro-3-(8-chloroquinazolin-4-yl)phenol (145 mg, 0.5 mmol), 4-fluoro-2-(methylsulfonyl)benzonitrile (120 mg, 0.6 mmol), and potassium carbonate (105 mg, 0.75 mmol) in dimethylacetamide (2 mL) was heated at 40° C. under a nitrogen atmosphere. After 16 h, the reaction was partitioned between ethyl acetate (40 mL) and water (20 mL). The layers were separated and the organic layer was washed with water (6×20 mL) and brine (20 mL). The combined extracts were dried (Na₂SO₄) and concentrated in vacuo. Chromatography on silica gel (0:100 to 25:75 E:H gradient) gave the title compound as a white solid. MS (ESI) m/z 470.1; HRMS: calcd for C₂₂H₁₃Cl₂N₃O₃S+H⁺, 470.01274; found (ESI, [M+H]⁺ Obs'd), 470.0129.

### Example 2

### 4-{4-chloro-3-[8-(trifluoromethyl)quinazolin-4-yl]phenoxy}-2-(methylsulfonyl)benzonitrile

### Step 1: 8-(trifluoromethyl)quinazolin-4-ol

The title compound was prepare according to a similar procedure to that described in Example 1, Step 1 except using 3-trifluoromethylanthranilic acid in place of 2-amino-3-chlorobenzoic acid. MS (ES) m/z 214.8; HRMS: calcd for C₉H₅F₃N₂O+H⁺, 215.04267; found (ESI, [M+H]⁺ Obs'd), 215.0428.

### Step 2: 4-chloro-8-(trifluoromethyl)quinazoline

The title compound was prepared according to a similar procedure to that described in Example 1, Step 2 except using 8-(trifluoromethyl)quinazolin-4-ol in place of 8-chloroquinazolin-4(3H)-one. ¹H NMR (400 MHz; CDCl₃) δ 9.16 (1H, s), 8.48 (1H, d, J=8.4 Hz), 8.29 (1H, d, J=7.4 Hz), 7.77 (1H, t, J=7.9 Hz).

### Step 3: 4-(2-chloro-5-methoxyphenyl)-8-(trifluoromethyl)quinazoline

The title compound was prepared according to a similar procedure to that described in Example 1, Step 3 except using 4-chloro-8-(trifluoromethyl)quinazoline in place of 4,8-dichloroquinazoline. MS (ES) m/z 338.7; HRMS: calcd for C₁₆H₁₀ClF₃N₂O+H⁺, 339.05065; found (ESI, [M+H]⁺ Obs'd), 339.0510.

### Step 4: 4-chloro-3-[8-(trifluoromethyl)quinazolin-4-yl]phenol

The title compound was prepared according to a similar procedure to that described in Example 1, Step 4 except using 4-(2-chloro-5-methoxyphenyl)-8-(trifluoromethyl)quinazoline in place of 8-chloro-4-(2-chloro-5-methoxyphenyl)quinazoline. MS (ES) m/z 324.8; HRMS: calcd for C₁₅H₈ClF₃N₂O+H⁺, 325.03500; found (ESI, [M+H]⁺ Obs'd), 325.0355.

### Step 5: 4-[4-chloro-3-(8-chloroquinazolin-4-yl)phenoxyl-2-(methylsulfonyl)benzonitrile

The title compound was prepared according to a similar procedure to that described in Example 1, Step 5 except using 4-chloro-3-[8-(trifluoromethyl)quinazolin-4-yl]phenol in place of 4-chloro-3-(8-chloroquinazolin-4-yl)phenol. MS (ESI) m/z 504.1; HRMS: calcd for C₂₃H₁₃ClF₃N₃O₃S+H⁺, 504.03910; found (ESI, [M+H]⁺ Obs'd), 504.0392.

### Example 3

### 4-[(3-{4-chloro-3-[8-(trifluoromethyl)quinazolin-4-yl]phenoxy}phenyl)sulfonyl]-butanenitrile

A mixture of 4-chloro-3-[8-(trifluoromethyl)quinazolin-4-yl]phenol (1.3 g, 4.0 mmol), 4-[(3-bromophenyl)sulfonyl]butanenitrile (1.73 g, 6 mmol), Cs₂CO₃ (4.55 g, 3.5 mmol), CuI (304 mg, 1.6 mmol), and N,N′-dimethylglycine hydrochloride (420 mg, 3.0 mmol) in dioxane (12 mL) was stirred at 95° C. for 16 h. The reaction was cooled and partitioned between EtOAc (120 mL) and water (80 mL). The layers were separated and the organic layer was washed with 3N NH₄OH (30 mL), water (2×30 mL), and brine (60 mL). The organic layer was dried with Na₂SO₄ and concentrated in vacuo. Chromatography on silica gel eluting with EtOAc:Hex gradient of 0:100 to 40:60 afforded the title compound as a white foam-solid. MS (ESI) m/z 532.1; HRMS: calcd for C₂₅H₁₇ClF₃N₃O₃S+H⁺, 532.07040; found (ESI, [M+H]⁺ Obs'd), 532.0704.

### Example 4

### 4-({3-[4-chloro-3-(8-chloroquinazolin-4-yl)phenoxy]phenyl}sulfonyl)butanenitrile

The title compound was prepared according to a similar procedure to that described in Example 3 except using 4-chloro-3-(8-chloroquinazolin-4-yl)phenol in place of 4-chloro-3-[8-(trifluoromethyl)quinazolin-4-yl]phenol. MS (ESI) m/z 532.1; HRMS: calcd for C₂₅H₁₇ClF₃N₃O₃S+H⁺, 532.07040; found (ESI, [M+H]⁺ Obs'd), 532.0704.

### Example 5

### 4-[2-chloro-5-(3-{[3-(methylsulfonyl)propyl]sulfonyl}phenoxy)phenyl]-8-(trifluoro-methyl)quinazoline

Step 1: 3-[(3-{4-chloro-3-[8-(trifluoromethyl)quinazolin-4-yl]phenoxy}phenyl)sulfonyl]-propan-1-ol

The title compound was prepared according to a similar procedure to that described in Example 3 except using 3-[(3-bromophenyl)sulfonyl]propan-1-ol in place of 4-[(3-bromophenyl)sulfonyl]butanenitrile. MS (ESI) m/z 523.1; HRMS: calcd for C₂₄H₁₈ClF₃N₂O₄S+H⁺, 523.07006; found (ESI, [M+H]⁺ Obs'd), 523.0701.

### Step 2: 4-(2-chloro-5-{3-[(3-iodopropyl)sulfonyl]phenoxy}phenyl)-8-(trifluoromethyl)-quinazoline

To a 0° C. stirred mixture of 3-[(3-{4-chloro-3-[8-(trifluoromethyl)quinazolin-4-yl]phenoxy}phenyl)sulfonyl]propan-1-ol (370 mg, 0.71 mmol) and diisopropylethyl-amine (0.31 mL, 0.85 mmol) in DCM (5 mL) was added methanesulfonyl chloride (66 μL, 0.85 mmol). After 1 h, the reaction was partitioned between ethyl acetate (40 mL) and water (20 mL). The layers were separated and the organic layer was washed with citric acid (20 mL), water (2×20 mL) and brine (20 mL). The combined extracts were dried (Na₂SO₄) and concentrated in vacuo. The residue was dissolved in acetone (10 mL) and sodium iodide (1.50 g, 10 mmol) was added. The mixture was stirred for 4 h at 50° C. The mixture was partitioned between ethyl acetate (60 mL) and water (40 mL) and the layers were separated. The organic layer was washed with water (2×20 mL) and brine (20 mL). The combined extracts were dried (Na₂SO₄) and concentrated in vacuo. Chromatography on silica gel (0:100 to 25:75 E:H gradient) gave the title compound as a white foam. MS (ESI) m/z 633.0; HRMS: calcd for C₂₄H₁₇ClF₃IN₂O₃S+H⁺, 632.97180; found (ESI, [M+H]⁺ Obs'd), 632.9715.

### Step 3: 4-[2-chloro-5-(3-{[3-(methylsulfonyl)propyl]sulfonyl}phenoxy)phenyl]-8-(trifluoromethyl)-quinazoline

To a 0° C. stirred mixture of 4-(2-chloro-5-{3-[(3-iodopropyl)sulfonyl]phenoxy}-phenyl)-8-(trifluoromethyl)quinazoline (100 mg, 0.16 mmol) in DMF (5 mL) was added sodium thiomethoxide (22 mg, 0.32 mmol). The mixture was stirred for 30 min at 0° C. and then was warmed to rt. Oxone (975 mg, 1.60 mmol), NaHCO₃ (168 mg, 2 mmol), acetone (2 mL) and water (2 mL) were added and the mixture was stirred at rt for 16 h. The mixture was partitioned between ethyl acetate (40 mL) and water (20 mL) and the layers were separated. The organic layer was washed with water (2×20 mL) and brine (20 mL). The combined extracts were dried (Na₂SO₄) and concentrated in vacuo. Chromatography on silica gel (0:100 to 25:75 E:H gradient) gave the title compound as a white foam. MS (ESI) m/z 585.1; HRMS: calcd for C₂₄H₁₈ClF₃N₂O₄S+H⁺, 523.07006; found (ESI, [M+H]⁺ Obs'd), 523.0701.

### Example 6

### 4-[4-chloro-3-(8-chloroquinazolin-4-yl)phenoxy]-2-(methylsulfonyl)benzamide

Concentrated sulfuric acid (3 mL) was added to solid 4-[4-chloro-3-(8-chloro-quinazolin-4-yl)phenoxy]-2-(methylsulfonyl)benzonitrile (80 mg, 0.17 mmol) and the red mixture was stirred at rt for 4 h. The solution was slowly dropped into stirred ice-water and the resulting precipitate was collected and washed with water. The white filter-cake was dried under high vacuum. MS (ESI) m/z 488.1; HRMS: calcd for C₂₂H₁₅Cl₂N₃O₄S+H⁺, 488.02331; found (ESI, [M+H]⁺ Obs'd), 488.0233.

### Example 7

### 4-{4-chloro-3-[8-(trifluoromethyl)quinazolin-4-yl]phenoxy}-2-(methylsulfonyl)benzamide

The title compound was prepared according to a similar procedure to that described in Example 6 except using 4-{4-chloro-3-[8-(trifluoromethyl)quinazolin-4-yl]phenoxy}-2-(methylsulfonyl)benzonitrile in place of 4-[4-chloro-3-(8-chloroquinazolin-4-yl)phenoxy]-2-(methylsulfonyl)benzonitrile. MS (ESI) m/z 522.1; HRMS: calcd for C₂₃H₁₅ClF₃N₃O₄S+H⁺, 522.04966; found (ESI, [M+H]⁺ Obs'd), 522.0496.

### Example 8

### 4-({3-[4-chloro-3-(8-chloroquinazolin-4-yl)phenoxy]phenyl}sulfonyl)butanamide

The title compound was prepared according to a similar procedure to that described in Example 6 except using 4-4-({3-[4-chloro-3-(8-chloroquinazolin-4-yl)phenoxy]phenyl}sulfonyl)butanenitrile in place of 4-[4-chloro-3-(8-chloroquinazolin-4-yl)phenoxy]-2-(methylsulfonyl)benzonitrile. MS (ESI) m/z 516.1; HRMS: calcd for C₂₄H₁₉Cl₂N₃O₄S+H⁺, 516.05461; found (ESI, [M+H]⁺ Obs'd), 516.0542.

### Example 9

### 4-[(3-{4-chloro-3-[8-(trifluoromethyl)quinazolin-4-yl]phenoxy}phenyl)sulfonyl]-butanamide

The title compound was prepared according to a similar procedure to that described in Example 6 except using 4-4-[(3-{4-chloro-3-[8-(trifluoromethyl)quinazolin-4-yl]phenoxy}phenyl)sulfonyl]butanenitrile in place of 4-[4-chloro-3-(8-chloroquinazolin-4-yl)phenoxy]-2-(methylsulfonyl)benzonitrile. MS (ESI) m/z 550.1; HRMS: calcd for C₂₅H₁₉ClF₃N₃O₄S+H⁺, 550.08096; found (ESI, [M+H]⁺ Obs'd), 550.0807.

### Example 10

### ({3-[3-(8-chloroquinazolin-4-yl)-4-fluorophenoxy]phenyl}sulfonyl)acetonitrile

### Step 1: 8-chloro-4-(2-fluoro-5-methoxyphenyl)quinazoline

The title compound was prepared according to a similar procedure to that described in Example 1, Step 3 except using 2-fluoro-5-methoxyphenylboronic acid in place of 2-chloro-5-methoxyphenylboronic acid. MS (ESI) m/z 289.0; HRMS: calcd for C₁₅H₁₀ClFN₂O+H⁺, 289.05384; found (ESI, [M+H]⁺ Obs'd), 289.0541.

### Step 2: 3-(8-chloroquinazolin-4-yl)-4-fluorophenol

The title compound was prepared according to a similar procedure to that described in Example 1, Step 4 except using 8-chloro-4-(2-fluoro-5-methoxyphenyl)quinazoline in place of 8-chloro-4-(2-chloro-5-methoxyphenyl)quinazoline. MS (ESI) m/z 275.0; HRMS: calcd for C₁₄H₈ClFN₂O+H⁺, 275.03819; found (ESI, [M+H]⁺ Obs'd), 275.0387.

### Step 3: ({3-[3-(8-chloroquinazolin-4-yl)-4-fluorophenoxylphenyl}sulfonyl)acetonitrile

The title compound was prepared according to a similar procedure to that described in Example 3 except using 3-(8-chloroquinazolin-4-yl)-4-fluorophenol in place of 4-chloro-3-[8-(trifluoromethyl)quinazolin-4-yl]phenol and [(3-bromophenyl)sulfonyl]acetonitrile in place of 4-[(3-bromophenyl)sulfonyl]butanenitrile. MS (ESI) m/z 454.0; HRMS: calcd for C₂₂H₁₃ClFN₃O₃S+H⁺, 454.04229; found (ESI, [M+H]⁺ Obs'd), 454.0424.

### Example 11

### [(3-{4-chloro-3-[8-(trifluoromethyl)quinazolin-4-yl]phenoxy}phenyl)sulfonyl]-acetonitrile

The title compound was prepared according to a similar procedure to that described in Example 3 except using [(3-bromophenyl)sulfonyl]acetonitrile in place of 4-[(3-bromophenyl)sulfonyl]butanenitrile. MS (ESI) m/z 504.1; HRMS: calcd for C₂₃H₁₃ClF₃N₃O₃S+H⁺, 504.03910; found (ESI, [M+H]⁺ Obs'd), 504.0387.

### Example 14

### 2-[(3-{4-chloro-3-[8-(trifluoromethyl)quinazoliyl]phenoxy}phenyl)sulfonyl]-acetamide

A solution of [(3-{4-chloro-3-[8-(trifluoromethyl)quinazolin-4-yl]phenoxy}phenyl)- sulfonyl]acetonitrile (60 mg, 0.12 mmol) in THF (4mL), 30% H₂O₂ (4 mL), and NH₄OH (4 mL) was stirred overnight at room temperature. The reaction mixture was poured into water and extracted with EtOAc. The extracts were dried with MgSO₄ and the solvent was removed in vacuo and the residue was purified by chromatography on SiO₂ eluting with a 2:98 to 100:0 EtOAc:Hex gradient. The product was isolated as a white solid. MS (ESI) m/z 522.1.

### Example 15

### [(3-{4-chloro-3-[8-(trifluoromethyl)quinazolin-4-yl]phenoxy}phenyl)sulfonyl]acetic acid

A mixture of 2-[(3-{4-chloro-3-[8-(trifluoromethyl)quinazoline-4-yl]phenoxy}phenyl)sulfonyl]acetamide (30 mg, 0.06 mmol), acetic acid (2 mL), and concentrated HCl (2 mL) was heated to reflux for 1 hr. Ice was added to the reaction mixture and the resulting precipitate was filtered and dried over vacuum to afford the pure title compound as a white solid. MS (ESI) m/z 523.1.

### Example 16

### 3-[4-chloro-3-(8-chloroquinazolin-4-yl)phenoxy]-N-methylbenzenesulfonamide

### Step 1: 3-bromo-N-(4-methoxybenzyl)benzenesulfonamide

A stirred mixture of 3-bromobenzenesulfonyl chloride (5.11 g, 20.0 mmol) and triethylamine (3.07 mL, 22.0 mmol) in dichloromethane (100 mL) was treated with 4-methoxybenzylamine (2.85 mL, 22.0 mmol) over 5 min. After 2 h at ambient temperature, the reaction is treated with saturated aqueous NaHCO₃ (100 mL) and extracted with dichloromethane (2×50 mL). The combined extracts were dried (MgSO₄) and concentrated in vacuo to a solid. Chromatography eluting with a 30:70 to 50:50 E:H gradient affords the title compound as a white solid (6.18 g, R_{f}˜0.4 in 50:50 E:H). MS (ES−) m/z: 353.6.

### Step 2: 3-bromo-N-(4-methoxybenzyl)-N-methylbenzenesulfonamide

A solution of 3-bromo-N-(4-methoxybenzyl)benzenesulfonamide (2.49 g, 7.00 mmol) in DMF (20 mL) is treated with 60% NaH in oil (336 mg, 8.4 mmol). Gas evolved. After 10 min, methyl iodide (0.66 mL, 10.5 mmol) is added and the reaction is stirred at ambient temperature overnight. The reaction is poured into water (60 mL) and extracted with ethyl acetate (2×40 mL). The extracts are dried (MgSO4), concentrated in vacuo, and chromatographed using a 20:80 to 50:50 E:H gradient to afford the title compound as a white solid (2.21 g, Rf˜0.5 in 50:50 E:H). MS (ESI Na) m/z 392.0 [M+Na]

### Step 3: 3-[4-chloro-3-(8-chloroquinazolin-4-yl)phenoxy]-N-(4-methoxybenzyl)-N-methylbenzenesulfonamide

A mixture of 4-chloro-3-(8-chloroquinazolin-4-yl)phenol (73 mg, 0.25 mmol), 3-bromo-N-(4-methoxybenzyl)-N-methylbenzenesulfonamide (139 mg, 0.375 mmol), CuI (10 mg, 0.053 mmol), Me₂NCH₂CO₂H hydrochloride (14 mg, 0.011 mmol), and cesium carbonate (245 mg, 0.75 mmol) in 1,4-dioxane (5.0 mL) was heated at reflux under nitrogen. After 20 h, the reaction was diluted with ethyl acetate (15 mL), filtered through Celite, and concentrated in vacuo. The residue was chromatographed on silica gel using a 10:90 to 45:55 E:H gradient and then by reverse phase chromatography using a 0:100 to 100:0 acetonitrile:water gradient to afford the title compound as a white solid (78 mg, R_{f}˜0.15 in 50:50 E:H). MS (ESI) m/z 580.1

### Step 4: 3-[4-chloro-3-(8-chloroquinazolin-4-yl)phenoxyl-N-methylbenzenesulfonamide

To a stirred mixture of 3-[4-chloro-3-(8-chloroquinazolin-4-yl)phenoxy]-N-(4-methoxybenzyl)-N-methylbenzenesulfonamide in dichloromethane (2.0 mL) was added trifluoroacetic acid (1.0 mL). After 18 h, the reaction was concentrated in vacuo, dissolved in dichloromethane (5 mL), and washed with saturated aqueous NaHCO₃. The organic layer was dried (MgSO₄), concentrated in vacuo, and purifed by chromatography eluting with a 20:80 to 70:30 E:H gradient to afford the title compound as a white solid (50 mg). MS (ESI) m/z 460.1; HRMS: calcd for C₂₁H₁₅Cl₂N₃O₃S+H+, 460.0284; found (ESI, [M+H]+ Obs'd), 460.0270.

### Example 17

### 3-[4-chloro-3-(8-chloroquinazolin-4-yl)phenoxy]benzenesulfonamide

### Step 1: 3-bromo-N,N-bis(4-methoxybenzyl)benzenesulfonamide

A solution of 3-bromo-N-(4-methoxybenzyl)benzenesulfonamide (3.56 g, 10.0 mmol) in DMF (20 mL) is treated with 60% NaH in oil (440 mg, 11.0 mmol). Gas evolved. After 20 min, 4-methoxybenzyl chloride (1.63 mL, 12.0 mmol) is added and the reaction is stirred at ambient temperature for 5 h. The reaction is poured into water (60 mL) resulting in a white precipitate. The precipitate is filtered off, washed with water, and dried in vacuo to afford the title compound as a white solid (4.92 g, R_{f}˜0.5 in E:H). MS (ESI Na) m/z 498.0 [M+Na]

### Step 2: 3-[4-chloro-3-(8-chloroquinazolin-4-yl)phenoxy]-N,N-bis(4-methoxybenzyl)benzene-sulfonamide

The title compound was prepared using a procedure analogous to that described in Example 16, step 3, except using 4-chloro-3-(8-chloroquinazolin-4-yl)phenol and 3-bromo-N,N-bis(4-methoxybenzyl)benzenesulfonamide to afford the title compound as a white solid from a foam. MS (ESI) m/z 686.2.

### Step 3: 3-[4-chloro-3-(8-chloroquinazolin-4-yl)phenoxy]benzenesulfonamide

The title compound was prepared using a procedure analogous to that described in Example 16, step 4, except using 3-[4-chloro-3-(8-chloroquinazolin-4-yl)phenoxyl-N,N-bis(4-methoxybenzyl)benzenesulfonamide to afford the title compound as an off-white solid. MS (ESI) m/z 446.1. HRMS: calcd for C₂₀H₁₃Cl₂N₃O₃S+H⁺, 446.01274; found (ESI, [M+H]⁺ Obs'd), 446.0123.

### Example 18

### 3-{4-chloro-3-[8-(trifluoromethyl)quinazolin-4-yl]phenoxyl-N-methylbenzene-sulfonamide

### Step 1: 3-{4-chloro-3-[8-(trifluoromethyl)quinazolin-4-yl]phenoxyl-N-(4-methoxybenzyl)-N-methylbenzenesulfonamide

The title compound was prepared using a procedure analogous to that described in Example 16, step 3, except using 4-chloro-3-[8-(trifluoromethyl)quinazolin-4-yl]phenol and 3-bromo-N-(4-methoxybenzyl)-N-methylbenzenesulfonamide to afford the title compound as a viscous oil solidifying to a glass. MS (ESI) m/z 614.2.

### Step 2: 3-{4-chloro-3-[8-(trifluoromethyl)quinazolin-4-yl]phenoxyl-N-methylbenzene-sulfonamide

The title compound was prepared using a procedure analogous to that described in Example 16, step 4, except using 3-{4-chloro-3-[8-(trifluoromethyl)quinazolin-4-yl]phenoxy}-N-(4-methoxybenzyl)-N-methylbenzenesulfonamide to afford the title compound as an off-white solid. MS (ESI) m/z 494.1. HRMS: calcd for C₂₂H₁₅ClF₃N₃O₃S+H⁺, 494.05475; found (ESI, [M+H]⁺ Obs'd), 494.0546.

### Example 19

### 3-{4-chloro-3-[8-(trifluoromethyl)quinazolin-4-yl]phenoxy}benzenesulfonamide

### Step 1: 3-{4-chloro-3-[8-(trifluoromethyl)quinazolin-4-yl]phenoxyl-N,N-bis(4-methoxybenzyl)-benzenesulfonamide

The title compound was prepared using a procedure analogous to that described in Example 16, step 3, except using 4-chloro-3-[8-(trifluoromethyl)quinazolin-4-yl]phenol and 3-bromo-N,N-bis(4-methoxybenzyl)benzenesulfonamide to afford the title compound as a viscous oil solidifying to a glass. MS (ESI) m/z 720.2.

### Step 2: 3-{4-chloro-3-[8-(trifluoromethyl)quinazolin-4-yl]phenox}benzenesulfonamide

The title compound was prepared using a procedure analogous to that described in Example 16, step 4, except using 3-{4-chloro-3-[8-(trifluoromethyl)quinazolin-4-yl]phenoxy}-N,N-bis(4-methoxybenzyl)benzenesulfonamide to afford the title compound as an off-white solid. MS (ESI) m/z 480.1; HRMS: calcd for C₂₁H₁₃ClF₃N₃O₃S+H⁺, 480.03910; found (ESI, [M+H]⁺ Obs'd), 480.0387.

### Example 20

### 3-[3-(8-chloroquinazolin-4-yl)-4-fluorophenoxy]benzenesulfonamide

### Step 1: 3-[3-(8-chloroquinazolin-4-yl)-4-fluorophenoxy]-N,N-bis(4-methoxybenzyl)-benzene-sulfonamide

The title compound was prepared using a procedure analogous to that described in Example 16, step 3, except using 3-(8-chloroquinazolin-4-yl)-4-fluorophenol and 3-bromo-N,N-bis(4-methoxybenzyl)benzenesulfonamide to afford the title compound as a white solid. MS (ESI) m/z 670.2; HRMS: calcd for C₃₆H₂₉ClFN₃O₅S+H⁺, 670.15732; found (ESI, [M+H]⁺ Obs'd), 670.1572.

### Step 2: 3-[3-(8-chloroquinazolin-4-yl)-4-fluorophenoxy]benzenesulfonamide

The title compound was prepared using a procedure analogous to that described in Example 16, step 4, except using 3-[3-(8-chloroquinazolin-4-yl)-4-fluorophenoxy]-N,N-bis(4-methoxybenzyl)benzenesulfonamide to afford the title compound as an off-white solid. MS (ESI) m/z 430.1; HRMS: calcd for C₂₀H₁₃ClFN₃O₃S+H⁺, 430.04229; found (ESI, [M+H]⁺ Obs'd), 430.0423.

### Example 21

### 3-{4-fluoro-3-[8-(trifluoromethyl)quinazolin-4-yl]phenoxy}benzenesulfonamide

### Step 1: 3-{4-fluoro-3-[8-(trifluoromethyl)quinazolin-4-yl]phenoxyl-N,N-bis(4-methoxybenzyl)benzenesulfonamide

The title compound was prepared using a procedure analogous to that described in Example 16, step 3, except using 4-fluoro-3-[8-(trifluoromethyl]quinazolin-4-yl)phenol and 3-bromo-N,N-bis(4-methoxybenzyl)benzenesulfonamide to afford the title compound as a white solid. MS (ESI) m/z 704.2; HRMS: calcd for C₃₇H₂₉F₄N₃O₅S+H+, 704.18368; found (ESI, [M+H]+ Obs'd), 704.1842.

### Step 2: 3-{4-fluoro-3-[8-(trifluoromethyl)quinazolin-4-yl]phenoxy}benzenesulfonamide

The title compound was prepared using a procedure analogous to that described in Example 16, step 4, except using 3-{4-fluoro-3-[8-(trifluoromethyl)quinazolin-4-yl]phenoxy}-N,N-bis(4-methoxybenzyl)benzenesulfonamide to afford the title compound as an off-white solid. MS (ESI) m/z 464.1; HRMS: calcd for C₂₁H₁₃F₄N₃O₃S+H+, 464.06865; found (ESI, [M+H]+ Obs'd), 464.0690.

### Example 22

### 3-{4-fluoro-3-[8-(trifluoromethyl)quinazolin-4-yl]phenoxyl-N-methylbenzene-sulfonamide

### Step 1: 3-{4-fluoro-3-[8-(trifluoromethyl)quinazolin-4-yl]phenoxyl-N-(4-methoxybenzyl)-N-methylbenzenesulfonamide

The title compound was prepared using a procedure analogous to that described in Example 16, step 3, except using 4-fluoro-3-[8-(trifluoromethyl]quinazolin-4-yl)phenol and 3-bromo-N-(4-methoxybenzyl)-N-methylbenzenesulfonamide to afford the title compound as a white solid. MS (ESI) m/z 598.2; HRMS: calcd for C₃₀H₂₃F₄N₃O₄S+H⁺, 598.14182; found (ESI, [M+H⁺ Obs'd), 598.1419.

### Step 2: 3-{4-fluoro-3-[8-(trifluoromethyl)quinazolin-4-yl]phenoxyl-N-methylbenzene-sulfonamide

The title compound was prepared using a procedure analogous to that described in Example 16, step 4, except using 3-{4-fluoro-3-[8-(trifluoromethyl)quinazolin-4-yl]phenoxy}-N-(4-methoxybenzyl)-N-methylbenzenesulfonamide to afford the title compound as a white solid from a foam. MS (ESI) m/z 478.1; HRMS: calcd for C₂₂H₁₅F₄N₃O₃S+H⁺, 478.08430; found (ESI, [M+H]⁺ Obs'd), 478.0845.

### Example 23

### 3-[3-(8-chloroquinazolin-4-yl)-4-fluorophenoxy]-N-methylbenzenesulfonamide

### Step 1: 3-[3-(8-chloroquinazolin-4-yl)-4-fluorophenoxyl-N-(4-methoxybenzyl)-N-methylbenzenesulfonamide

The title compound was prepared using a procedure analogous to that described in Example 16, step 3, except using 3-(8-chloroquinazolin-4-yl)-4-fluorophenol and 3-bromo-N-(4-methoxybenzyl)-N-methylbenzenesulfonamide to afford the title compound as a white solid from a glass. MS (ESI) m/z 564.1; HRMS: calcd for C₂₉H₂₃ClFN₃O₄S+H⁺, 564.1155; found (ESI, [M+H]⁺ Obs'd), 564.1157.

### Step 2: 3-[3-(8-chloroquinazolin-4-yl)-4-fluorophenoxyl-N-methylbenzenesulfonamide

The title compound was prepared using a procedure analogous to that described in Example 16, step 4, except using 3-[3-(8-chloroquinazolin-4-yl)-4-fluorophenoxy]-N-(4-methoxybenzyl)-N-methylbenzenesulfonamide to afford the title compound as an off-white solid. MS (ESI) m/z 444.1; HRMS: calcd for C₂₁H₁₅ClFN₃O₃S+H⁺, 444.0579; found (ESI, [M+H]⁺ Obs'd), 444.0582.

### Example 24

### 3-[(3-{4-fluoro-3-[8-(trifluoromethyl)quinazolin-4-yl]phenoxy}phenyl)sulfonyl]propan-1-ol

### Step 1: 4-(2-fluoro-5-methoxyphenyl)-8-(trifluoromethyl)quinazoline

The title compound was preparedaccording to a similar procedure to that described in Example 1, Step 3 except using 2-fluoro-5-methoxyphenylboronic acid in place of 2-chloro-5-methoxyphenylboronic acid. MS (ES) m/z 323.0; HRMS: calcd for C₁₆H₁₀F₄N₂O+H⁺, 323.08020; found (ESI, [M+H]⁺ Obs'd), 323.0804.

### Step 2: 4-fluoro-3-[8-(trifluoromethyl)quinazolin-4-yl]phenol

The title compound was prepared according to a similar procedure to that described in Example 1, Step 4 except using 4-(2-fluoro-5-methoxyphenyl)-8-(trifluoromethyl)quinazoline in place of 8-chloro-4-(2-chloro-5-methoxyphenyl)quinazoline. HRMS: calcd for C₁₅H₈F₄N₂O+H⁺, 309.06455; found (ESI, [M+H]⁺ Obs'd), 309.0650.

### Step 3: 3-[(3-bromophenyl)sulfonyl]propan-1-ol

3-Bromo-1-propanol (13.9 g, 0.1 mol) in DMF (10 mL) was added dropwise to a stirred mixture of 3-bromothiophenol (18.9 g, 0.1 mol) and Cs₂CO₃ (65.2 g, 0.2 mol) in DMF (50 mL) at room temperature. After 30 minutes the reaction was diluted with EtOAc and water. The layers were separated and the organic layer was washed with water and brine. The organic layer was dried with Na₂SO₄ and concentrated. The residue was dissolved in acetic acid (100 mL) and hydrogen peroxide (˜30% in water, 150 mL) was added slowly. The reaction mixture was heated to ˜70° C. for 2 hours, treated with iced water, and extracted with EtOAc. The extracts were dried with Na₂SO₄ and concentrated. Chromatography on silica gel eluting with EtOAc:Hex gradient of 0:100 to 100:0 afforded the title compound as a white foam-solid.

### Step 4: 3-[(3-{4-fluoro-3-[8-(trifluoromethyl)quinazolin-4-yl]phenoxy}phenyl)sulfonyl]propan-1-ol

The title compound was prepared according to a similar procedure to that described in Example 3 except using 4-fluoro-3-[8-(trifluoromethyl)quinazolin-4-yl]phenol in place of 4-chloro-3-[8-(trifluoromethyl)quinazolin-4-yl]phenol and 3-[(3-bromophenyl)sulfonyl]propan-1-ol in place of 4-[(3-bromophenyl)sulfonyl]butanenitrile.

MS (ESI) m/z 507.2; HRMS: calcd for C₂₄H₁₈F₄N₂O₄S+H⁺, 507.09962; found (ESI, [M+H]⁺ Obs'd), 507.1000.

### Example 25

### 4-[(3-{4-fluoro-3-[8-(trifluoromethyl)quinazolin-4-yl]phenoxy}phenyl)sulfonyl]butanenitrile

### Step 1: 3-[(3-{4-fluoro-3-[8-(trifluoromethyl)quinazolin-4-yl]phenoxy}phenyl)sulfonyl]propyl methanesulfonate

A mixture of 3-[(3-{4-fluoro-3-[8-(trifluoromethyl)quinazolin-4-yl]phenoxy}phenyl)sulfonyl]propan-1-ol (0.10 g, 0.2 mmol), methanesulfonyl chloride (0.31 g, 2.60 mmol), and triethylamine (0.5 mL, 3.6 mmol) in 5 mL of DCM was stirred for 30 minutes at rt. The solution was concentrated and the residue was purified by silica gel chromatography (EtOAc:Hex gradient of 0:100 to 100:0) to give the title compound as a white foam solid. MS (ESI) m/z 585.1.

### Step 2: 4-[(3-{4-fluoro-3-[8-(trifluoromethyl)quinazolin-4-yl]phenoxy}phenyl)sulfonyl]butanenitrile

A mixture of 3-[(3-{4-fluoro-3-[8-(trifluoromethyl)quinazolin-4-yl]phenoxy}phenyl)sulfonyl]propyl methanesulfonate (0.09 g, 0.15 mmol), potassium cyanide (0.5 g, 7.7 mmol) in DMF (4 mL) was stirred at room temperature overnight. The reaction mixture was poured into water and extracted with EtOAc. The extracts were dried with Na₂SO₄ and concentrated. Chromatography on silica gel eluting with EtOAc:Hex gradient of 0:100 to 100:0 afforded the title compound as a white solid. MS (ESI) m/z 516.1; HRMS: calcd for C₂₅H₁₇F₄N₃O₃S+H+, 516.09995; found (ESI, [M+H]+ Obs'd), 516.1004.

### Example 26

### 4-[(3-{4-fluoro-3-[8-(trifluoromethyl)quinazolin-4-yl]phenoxy}phenyl)sulfonyl]butanamide

A mixture of 4-[(3-{4-fluoro-3-[8-(trifluoromethyl)quinazolin-4-yl]phenoxy}phenyl)sulfonyl]butanenitrile (0.06 g, 0.12 mmol), hydrogen peroxide (˜30% in water, 4 mL), ammonium hydroxide (˜28% in water, 4 mL) and THF (4 mL) was stirred for 40 min at rt. The reaction mixture was poured into water and extracted with EtOAc. The extracts were dried with Na₂SO₄ and concentrated. Chromatography on silica gel eluting with EtOAc:Hex gradient of 0:100 to 100: 0 afforded the title compound as a white solid. MS (ESI) m/z 534.2; HRMS: calcd for C₂₅H₁₉F₄N₃O₄S+H⁺, 534.11052; found (ESI, [M+H]⁺ Obs'd), 534.1106.

### Example 27

### 4-[(3-{4-fluoro-3-[8-(trifluoromethyl)quinazolin-4-yl]phenoxy}phenyl)sulfonyl]butanoic acid

A mixture of 4-[(3-{4-fluoro-3-[8-(trifluoromethyl)quinazolin-4-yl]phenoxy}phenyl)sulfonyl]butanamide (0.03 g, 0.056 mmol), acetic acid (2 mL), hydrochloric acid, concentrated, 3 mL) was heated to reflux for 30 minutes. Ice was added to the reaction mixture and the resulting precipitate was collected and dried over vacuum to offorded the title compound as a white solid. MS (ESI) m/z 535.1; HRMS: calcd for C₂₅H₁₈F₄N₂O₅S+H⁺, 535.09453; found (ESI, [M+H]⁺ Obs'd), 535.0949.

### Example 28

### 3-[(3-{4-fluoro-3-[8-(trifluoromethyl)quinazolin-4-yl]phenoxy}phenyl)sulfonyl]propan-1-amine

A mixture of 3-[(3-{4-fluoro-3-[8-(trifluoromethyl)quinazolin-4-yl]phenoxy}phenyl)sulfonyl]propyl methanesulfonate (0.05 g, 0.15 mmol), ammonia (7 N in methanol, 5 mL) in THF (3 mL) was heated in a sealed tube at 60° C. overnight. The reaction mixture was concentrated and purified by silica gel chromatography (DCM:methanol with ˜5% ammonia, 100:0 to 50:50) affording the title compound as a gummy solid. MS (ESI) m/z 506.2; HRMS: calcd for C₂₄H₁₉F₄N₃O₃S+H⁺, 506.11560; found (ESI, [M+H]⁺ Obs'd), 506.1160.

### Example 29

### N-{3-[(3-{4-fluoro-3-[8-(trifluoromethyl)quinazolin-4-yl]phenoxy}phenyl)sulfonyl]propyl}-methanesulfonamide

A mixture of 3-[(3-{4-fluoro-3-[8-(trifluoromethyl)quinazolin-4-yl]phenoxy}phenyl)sulfonyl]propan-1-amine (0.018 g, 0.035 mmol), methanesulfonyl chloride (0.015 g, 0.13 mmol), and triethylamine (0.2 mL, 0.15 mmol) in 2 mL of methylene chloride was stirred at room temperature for 30 minutes. The solution was concentrated and the residue was purified by reverse phase HPLC (CH₃CN/water) to give the title compound as a white foam solid. MS (ESI) m/z 584.1; HRMS: calcd for C₂₅H₂₁F₄N₃O₅S₂+H⁺, 584.09315; found (ESI, [M+H]⁺ Obs'd), 584.0932.

### Example 30

### 4-[(3-{4-fluoro-3-[8-(trifluoromethyl)quinazolin-4-yl]phenoxy}phenyl)sulfonyl]butan-1-ol

### Step 1: 4-[(3-bromophenyl)sulfonyl]butan-1-ol

The title compound was prepared according to a similar procedure to that described in Example 24, Step 3 but using 4-bromo-1-butanol in place of 3-bromo-1-propanol. MS (ESI) m/z 293.0.

### Step 2: 4-[(3-{4-fluoro-3-[8-(trifluoromethyl)quinazolin-4-yl]phenoxy}phenyl)sulfonyl]butan-1-ol

The title compound was prepared according to a similar procedure to that described in Example 3 but using 4-[(3-bromophenyl)sulfonyl]butan-1-ol in place of 3-[(3-bromophenyl)sulfonyl]propan-1-ol and 4-fluoro-3-[8-(trifluoromethyl)quinazolin-4-yl]phenol in place of 4-chloro-3-[8-(trifluoromethyl)quinazolin-4-yl]phenol. MS (ESI) m/z 521.2; HRMS: calcd for C₂₅H₂₀F₄N₂O₄S+H⁺, 521.11527; found (ESI, [M+H]⁺ Obs'd), 521.1155.

### Example 31

### 5-[(3-{4-fluoro-3-[8-(trifluoromethyl)quinazolin-4-yl]phenoxy}phenyl)sulfonyl]pentanenitrile

### Step 1: 4-[(3-{4-fluoro-3-[8-(trifluoromethyl)quinazolin-4-yl]phenoxy}phenyl)sulfonyl]butyl methanesulfonate

The title compound was prepared according to a similar procedure to that described in Example 25, Step 1 but using 4-[(3-{4-fluoro-3-[8-(trifluoromethyl)quinazolin-4-yl]phenoxy}phenyl)sulfonyl]butan-1-ol in place of 3-[(3-{4-fluoro-3-[8-(trifluoromethyl)quinazolin-4-yl]phenoxy}phenyl)sulfonyl]propan-1-ol. MS (ESI) m/z 599.2; HRMS: calcd for C₂₆H₂₂F₄N₂O₆S₂+H⁺, 599.09282; found (ESI, [M+H]⁺ Obs'd), 599.0923.

### Step 2: 5-[(3-{4-fluoro-3-[8-(trifluoromethyl)quinazolin-4-yl]phenoxy}phenyl)sulfonyl]-pentanenitrile

The title compound was prepared according to a similar procedure to that described in Example 25, Step 2 but using 4-[(3-{4-fluoro-3-[8-(trifluoromethyl)quinazolin-4-yl]phenoxy}phenyl)sulfonyl]butyl methanesulfonate in place of 3-[(3-{4-fluoro-3-[8-(trifluoromethyl)quinazolin-4-yl]phenoxy}phenyl)sulfonyl]propyl methanesulfonate. MS (ESI) m/z 530.1; HRMS: calcd for C₂₆H₁₉F₄N₃O₃S+H⁺, 530.11560; found (ESI, [M+H]⁺ Obs'd), 530.1153.

### Example 32

### 4-[2-fluoro-5-(3-{[3-(methylsulfonyl)propyl]sulfonyl}phenoxy)phenyl]-8-(trifluoromethyl)-quinazoline

The title compound was prepared according to a similar procedure to that described in Example 25, step 2 but using sodium methanesulfinate in place of potassium cyanide. MS (ESI) m/z 569.1; HRMS: calcd for C₂₅H₂₀F₄N₂O₅S₂+H⁺, 569.08225; found (ESI, [M+H]⁺ Obs'd), 569.0812.

### Example 33

### 5-[(3-{4-fluoro-3-[8-(trifluoromethyl)quinazolin-4-yl]phenoxy}phenyl)sulfonyl]pentanamide

The title compound was prepared according to a similar procedure to that described in Example 26 but using 5-[(3-{4-fluoro-3-[8-(trifluoromethyl)quinazolin-4-yl]phenoxy}phenyl)-sulfonyl]pentanenitrile in place of 4-[(3-{4-fluoro-3-[8-(trifluoromethyl)quinazolin-4-yl]phenoxy}-phenyl)sulfonyl]butanenitrile. MS (ESI) m/z 548.2; HRMS: calcd for C₂₆H₂₁F₄N₃O₄S+H⁺, 548.12617; found (ESI, [M+H]⁺ Obs'd), 548.1264.

### Example 34

### 5-[(3-{4-fluoro-3-[8-(trifluoromethyl)quinazolin-4- yl]phenoxy}phenyl)sulfonyl]pentanoic acid

The title compound was preparedaccording to a similar procedure to that described in Example 27 but using 5-[(3-{4-fluoro-3-[8-(trifluoromethyl)quinazolin-4-yl]phenoxy}phenyl)sulfonyl]pentanamide in place of 4-[(3-{4-fluoro-3-[8-(trifluoromethyl)quinazolin-4-yl]phenoxy}phenyl)sulfonyl]butanamide. MS (ESI) m/z 549.2; HRMS: calcd for C₂₆H₂₀F₄N₂O₅S+H⁺, 549.11018; found (ESI, [M+I-1]⁺ Obs'd), 549.1101.

### Example 35

### 4-[(3-{4-fluoro-3-[8-(trifluoromethyl)quinazolin-4-yl]phenoxy}phenyl)sulfonyl]butan-1-amine

The title compound was prepared according to a similar procedure to that described in

Example 28 but using 4-[(3-{4-fluoro-3-[8-(trifluoromethyl)quinazolin-4- yl]phenoxy}phenyl)sulfonyl]butyl methanesulfonate in place of 3-[(3-{4-fluoro-3-[8-(trifluoromethyl)quinazolin-4-yl]phenoxy}phenyl)sulfonyl]propyl methanesulfonate. MS (ESI) m/z 520.2; HRMS: calcd for C₂₅H₂₁R₄N₃O₃S+H⁺, 520.13125; found (ESI, [M+H]⁺ Obs'd), 520.1309.

### Example 36

### N-{4-[(3-{4-fluoro-3-[8-(trifluoromethyl)quinazolin-4-yl]phenoxy}phenyl)sulfonyl]butyl]methanesulfonamide

The title compound was prepared according to a similar procedure to that described in Example 29 but using 4-[(3-{4-fluoro-3-[8-(trifluoromethyl)quinazolin-4-yl]phenoxy}phenyl)sulfonyl]butan-1-amine in place of 3-[(3-{4-fluoro-3-[8-(trifluoromethyl)quinazolin-4-yl]phenoxy}phenyl)sulfonyl]propan-1-amine. MS (ESI) m/z 598.2; HRMS: calcd for C₂₆H₂₃F₄N₃O₅S₂+H⁺, 598.10880; found (ESI, [M+H]⁺ Obs'd), 598.1087.

### Example 37

### 4-[2-fluoro-5-(3-{[4-(methylsulfonyl)butyl]sulfonyl}phenoxy)phenyl]-8-(trifluoromethyl)-quinazoline

The title compound was prepared according to a similar procedure to that described in Example 25, step 2 but using sodium methanesulfinate in place of potassium cyanide and 4-[(3-{4-fluoro-3-[8-(trifluoromethyl)quinazolin-4-yl]phenoxy}phenyl)sulfonyl]butyl methanesulfonate in place of 3-[(3-{4-fluoro-3-[8-(trifluoromethyl)quinazolin-4-yl]phenoxy}phenyl)sulfonyl]propyl methanesulfonate. HRMS: calcd for C₂₆H₂₂F₄N₂O₅S₂+H⁺, 583.09790; found (ESI, [M+H]⁺ Obs'd), 583.0981.

### Example 38

### 4-{4-fluoro-3-[8-(trifluoromethyl)quinazolin-4-yl]phenoxy}-2-(methylsulfonyl)benzonitrile

### Step 1: 4-fluoro-2-(methylthio)benzonitrile

In a round bottom flask under at room temperature under a nitrogen atmosphere was placed 2,4-difluorobenzonitrile (5.00 g, 35.971 mmol) in toluene (100 ml) upon which was added sodium methanethiolate (2.52 g, 35.97 mmol). This same resulting gray mixture was stirred at room temperature 18 h. The light gray mixture was filtered through a SiO₂ pad (5×3 cm), washed with EtOAc (50 ml) and concentrated in vacuo to afford a light yellow powder. This same yellow powder was subjected to SiO₂ chromatography elution with Hexane: EtOAc afforded the title compound as a white powder. MS (ESI) m/z 198.2; HRMS: calcd for C₈H₆FNO₂S+H⁺, 200.01760; found (ESI, [M+H]⁺ Obs'd), 200.0178.

### Step 2: 4-fluoro-2-(methylsulfonyl)benzonitrile

In a round bottom flask under at room temperature under a nitrogen atmosphere was placed 4-fluoro-2-(methylthio)benzonitrile (5.28 g, 31.617 mmol) in acetone (50 ml) and 0.5M NaHCO₃ (75 ml), upon which was added oxone (44.72 g, 72.72 mmol), with stifling, in ten equal portions over a 30 minute period. The resulting white mixture was stirred at room temperature for 18 h. The acetone was removed in vacuo and the resulting aqueous mixture was extracted with EtOAc (3×50 ml). Combination of all organics, drying over MgSO₄, filtration, and concentration yielded a white powder. This same white powder was subjected to SiO₂ chromatography elution with Hexane: EtOAc afforded the title compound as a white powder. HRMS: calcd for C₈H₆FNS+H⁺, 168.02777; found (ESI, [M+H]⁺ Obs'd), 168.0275.

### Step 3: 4-{4-fluoro-3-[8-(trifluoromethyl)quinazolin-4-yl]phenoxy}-2-(methylsulfonyl)benzonitrile

The title compound was prepared according to a similar procedure to that described in Example 1, step 5 but using 4-fluoro-3-[8-(trifluoromethyl)quinazolin-4-yl]phenol in place of 4-chloro-3-(8-chloroquinazolin-4-yl)phenol. MS (ESI) m/z 488.1; HRMS: calcd for C₂₃H₁₃F₄N₃O₃S+H⁺, 488.06865; found (ESI, [M+H]⁺ Obs'd), 488.0688.

### Example 39

### 4-{4-fluoro-3-[8-(trifluoromethyl)quinazolin-4-yl]phenoxy}-2-(methylsulfonyl)benzamide

The title compound was prepared according to a similar procedure to that described in Example 26 but using 4-{4-fluoro-3-[8-(trifluoromethyl)quinazolin-4-yl]phenoxy}-2-(methylsulfonyl)benzonitrile in place of 4-[(3-{4-fluoro-3-[8-(trifluoromethyl)quinazolin-4-yl]phenoxy}phenyl)sulfonyl]butanenitrile. MS (ESI) m/z 506.1; HRMS: calcd for C₂₃H₁₅F₄N₃O₄S+H⁺, 506.07921; found (ESI, [M+H]⁺ Obs'd), 506.0788.

### Example 40

### 4-{4-fluoro-3-[8-(trifluoromethyl)quinazolin-4-yl]phenoxy}-2-(methylsulfonyl)benzoic acid

The title compound was prepared according to a similar procedure to that described in Example 27 but using 4-{4-fluoro-3-[8-(trifluoromethyl)quinazolin-4-yl]phenoxy}-2-(methylsulfonyl)benzamide in place of 4-[(3-{4-fluoro-3-[8-(trifluoromethyl)quinazolin-4-yl]phenoxy}phenyl)sulfonyl]butanamide. MS (ESI) m/z 507.1; HRMS: calcd for C₂₃H₁₄F₄N₂O₅S+H⁺, 507.06323; found (ESI, [M+H]⁺ Obs'd), 507.0630.

### Example 41

### 4-[3-(8-chloroquinazolin-4-yl)-4-fluorophenoxy]-2-(methylsulfonyl)benzonitrile

The title compound was prepared according to a similar procedure to that described in Example 1, step 5 but using 4-fluoro-3-(8-chloroquinazolin-4-yl)phenol in place of 4-chloro-3-(8-chloroquinazolin-4-yl)phenol. MS (ESI) m/z 454.1; HRMS: calcd for C₂₂H₁₃ClFN₃O₃S+H⁺, 454.04229; found (ESI, [M+H]⁺ Obs'd), 454.0419.

### Example 42

### 4-[3-(8-chloroquinazolin-4-yl)-4-fluorophenoxy]-2-(methylsulfonyl)benzamide

The title compound was prepared according to a similar procedure to that described in Example 6 except using 4-[3-(8-chloroquinazolin-4-yl)-4-fluorophenoxy]-2-(methylsulfonyl)benzonitrile in place of 4-[4-chloro-3-(8-chloroquinazolin-4-yl)phenoxy]-2-(methylsulfonyl)benzonitrile.

MS (ESI) m/z 472.1; HRMS: calcd for C₂₂H₁₅ClFN₃O₄S+H⁺, 472.05286; found (ESI, [M+H]+ Obs'd), 472.0526.

### Example 43

### 4-({3-[3-(8-chloroquinazolin-4-yl)-4-fluorophenoxy]phenyl}sulfonyl)-N-methylbutan-1-amine

The title compound was prepared according to a similar procedure to that described in Example 28 but using 4-(3-(3-(8-chloroquinazolin-4-yl)-4-fluorophenoxy)phenylsulfonyl)butyl methanesulfonate in place of 3-[(3-{4-fluoro-3-[8-(trifluoromethyl)quinazolin-4-yl]phenoxy}phenyl)sulfonyl]propyl methanesulfonate and methylamine in place of ammonia. HRMS: calcd for C₂₅H₂₃ClFN₃O₃S+H⁺, 500.12054; found (ESI, [M+H]⁺ Obs'd), 500.1205.

### Example 44

### 3-({3-[3-(8-chloroquinazolin-4-yl)-4-fluorophenoxy]phenyl}sulfonyl)-N-methylpropan-1-amine

The title compound was prepared according to a similar procedure to that described in Example 28 but using 4-(3-(3-(8-chloroquinazolin-4-yl)-4-fluorophenoxy)phenylsulfonyl)propyl methanesulfonate in place of 3-[(3-{4-fluoro-3-[8-(trifluoromethyl)quinazolin-4-yl]phenoxy}phenyl)sulfonyl]propyl methanesulfonate and methylamine in place of ammonia. HRMS: calcd for C₂₄H₂₁ClFN₃O₃S+H+, 486.10489; found (ESI, [M+H]+ Obs'd), 486.1049.

### Example 45

### 4-({3-[3-(8-chloroquinazolin-4-yl)-4-fluorophenoxy]phenyl}sulfonyl)butan-1-amine

The title compound was prepared according to a similar procedure to that described in Example 28 but using 4-(3-(3-(8-chloroquinazolin-4-yl)-4-fluorophenoxy)phenylsulfonyl)butyl methanesulfonate in place of 3-[(3-{4-fluoro-3-[8-(trifluoromethyl)quinazolin-4-yl]phenoxy}phenyl)sulfonyl]propyl methanesulfonate. HRMS: calcd for C₂₄H₂₁ClFN₃O₃S+H⁺, 486.10489; found (ESI, [M+H]⁺ Obs'd), 486.1050.

### Example 46

### 3-({3-[3-(8-chloroquinazolin-4-yl)-4-fluorophenoxy]phenyl}sulfonyl)propan-1-amine

The title compound was prepared according to a similar procedure to that described in Example 28 but using 4-(3-(3-(8-chloroquinazolin-4-yl)-4-fluorophenoxy)phenylsulfonyl)propyl methanesulfonate in place of 3-[(3-{4-fluoro-3-[8-(trifluoromethyl)quinazolin-4-yl]phenoxy}phenyl)sulfonyl]propyl methanesulfonate. HRMS: calcd for C₂₃H₁₉ClFN₃O₃S+H⁺, 472.08924; found (ESI, [M+H]⁺ Obs'd), 472.0895.

### Example 47

### 4-({3-[3-(8-chloroquinazolin-4-yl)-4-fluorophenoxy]phenyl}sulfonyl)butanenitrile

The title compound was prepared according to a similar procedure to that described in Example 25, Step 2, but using 4-(3-(3-(8-chloroquinazolin-4-yl)-4-fluorophenoxy)phenylsulfonyl)butyl methanesulfonate in place of 3-[(3-{4-fluoro-3-[8-(trifluoromethyl)quinazolin-4-yl]phenoxy}phenyl)sulfonyl]propyl methanesulfonate. MS (ESI) m/z 482.1; HRMS: calcd for C₂₄H₁₇ClFN₃O₃S+H⁺, 482.07359; found (ESI, [M+H]⁺ Obs'd), 482.0736.

### Example 48

### N-[4-({3-[3-(8-chloroquinazolin-4-yl)-4-fluorophenoxy]phenyl}sulfonyl)butyl]-N-methylmethanesulfonamide

The title compound was prepared according to a similar procedure to that described in Example 29 but using 3-({3-[3-(8-chloroquinazolin-4-yl)-4-fluorophenoxy]phenyl}sulfonyl)-N-methylbutan-1-amine in place of 3-[(3-{4-fluoro-3-[8-(trifluoromethyl)quinazolin-4-yl]phenoxy}phenyl)sulfonyl]propan-1-amine. MS (ESI) m/z 578.1; HRMS: calcd for C₂₆H₂₅ClFN₃O₅S₂+H⁺, 578.09809; found (ESI, [M+H]⁺ Obs'd), 578.0983.

### Example 49

### N-[3-({3-[3-(8-chloroquinazolin-4-yl)-4-fluorophenoxy]phenyl}sulfonyl)propyl]-N-methylmethanesulfonamide

The title compound was prepared according to a similar procedure to that described in Example 29 but using 3-({3-[3-(8-chloroquinazolin-4-yl)-4-fluorophenoxy]phenyl}sulfonyl)-N-methylpropan-1-amine in place of 3-[(3-{4-fluoro-3-[8-(trifluoromethyl)quinazolin-4-yl]phenoxy}phenyl)sulfonyl]propan-1-amine. MS (ESI) m/z 564.1; HRMS: calcd for C₂₅H₂₃ClFN₃O₅S₂+H+, 564.08244; found (ESI, [M+H]+ Obs'd), 564.0825.

### Example 50

### N-[3-({3-[3-(8-chloroquinazolin-4-yl)-4-fluorophenoxy]phenyl}sulfonyl)propyl]methane-sulfonamide

The title compound was prepared according to a similar procedure to that described in Example 29 but using 3-({3-[3-(8-chloroquinazolin-4-yl)-4-fluorophenoxy]phenyl}sulfonyl)propan-1-amine in place of 3-[(3-{4-fluoro-3-[8-(trifluoromethyl)quinazolin-4-yl]phenoxy}phenyl)sulfonyl]propan-1-amine. MS (ESI) m/z 550.1; HRMS: calcd for C₂₄H₂₁ClFN₃O₅S₂+H⁺, 550.06679; found (ESI, [M+H]⁺ Obs'd), 550.0667.

### Example 51

### N-[4-({3-[3-(8-chloroquinazolin-4-yl)-4-fluorophenoxy]phenyl}sulfonyl)butyl]methane-sulfonamide

The title compound was prepared according to a similar procedure to that described in Example 29 but using 4-(3-(3-(8-chloroquinazolin-4-yl)-4-fluorophenoxy)phenylsulfonyl)butan-1-amine in place of 3-[(3-{4-fluoro-3-[8-(trifluoromethyl)quinazolin-4-yl]phenoxy}phenyl)sulfonyl]-propan-1-amine. MS (ESI) m/z 564.1; HRMS: calcd for C₂₅H₂₃ClFN₃O₅S₂+H+, 564.08244; found (ESI, [M+H]+ Obs'd), 564.0828.

### Example 52

### 5-({3-[3-(8-chloroquinazolin-4-yl)-4-fluorophenoxy]phenyl}sulfonyl)pentanenitrile

The title compound was prepared according to a similar procedure to that described in Example 25, Step 2 but using 4-[(3-{4-fluoro-3-[8-chloroquinazolin-4-yl]phenoxy}phenyl)sulfonyl]butyl methanesulfonate in place of 3-[(3-{4-fluoro-3-[8-(trifluoromethyl)quinazolin-4-yl]phenoxy}phenyl)sulfonyl]propyl methanesulfonate. HRMS: calcd for C₂₅H₁₉ClFN₃O₃S+H⁺, 496.08924; found (ESI, [M+H]⁺), 496.0897.

### Example 53

### 5-({3-[3-(8-chloroquinazolin-4-yl)-4-fluorophenoxy]phenyl}sulfonyl)pentanamide

The title compound was prepared according to a similar procedure to that described in Example 26 but using 5-[(3-{4-fluoro-3-[8-chloroquinazolin-4-yl]phenoxy}phenyl)sulfonyl]pentanenitrile in place of 4-[(3-{4-fluoro-3-[8-(trifluoromethyl)quinazolin-4-yl]phenoxy}phenyl)sulfonyl]butanenitrile. MS (ESI) m/z 514.2; HRMS: calcd for C₂₅H₂₁ClFN₃O₄S+H⁺, 514.09981; found (ESI, [M+H]⁺ Obs'd), 514.0998.

### Example 54

### 4-({3-[3-(8-chloroquinazolin-4-yl)-4-fluorophenoxy]phenyl}sulfonyl)butanamide

The title compound was prepared according to a similar procedure to that described in Example 26 but using 4-(3-(3-(8-chloroquinazolin-4-yl)-4-fluorophenoxy)phenylsulfonyl)butanenitrile in place of 4-[(3-{4-fluoro-3-[8-(trifluoromethyl)quinazolin-4-yl]phenoxy}phenyl)sulfonyl]butanenitrile. MS (ESI) m/z 500.2; HRMS: calcd for C₂₄H₁₉ClFN₃O₄S+H⁺, 500.08416; found (ESI, [M+H]⁺ Obs'd), 500.0843.

### Example 55

### 4-[3-(8-chloroquinazolin-4-yl)-4-fluorophenoxy]-2-(methylsulfonyl)benzoic acid

The title compound was prepared according to a similar procedure to that described in Example 27 but using 4-{4-fluoro-3-[8-chloroquinazolin-4-yl]phenoxy}-2-(methylsulfonyl)benzamide in place of 4-[(3-{4-fluoro-3-[8-(trifluoromethyl)quinazolin-4-yl]phenoxy}phenyl)sulfonyl]butanamide. MS (ESI) m/z 473.1; HRMS: calcd for C₂₂H₁₄ClFN₂O₅S+H⁺, 473.03687; found (ESI, [M+H]⁺ Obs'd), 473.0367.

### Example 56

### 5-({3-[3-(8-chloroquinazolin-4-yl)-4-fluorophenoxy]phenyl}sulfonyl)pentanoic acid

The title compound was prepared according to a similar procedure to that described in

Example 27 but using 5-[(3-{4-fluoro-3-[8-chloroquinazolin-4-yl]phenoxy}phenyl)sulfonyl]pentanamide in place of 4-[(3-{4-fluoro-3-[8-(trifluoromethyl)quinazolin-4-yl]phenoxy}phenyl)sulfonyl]butanamide. MS (ESI) m/z 515.1; HRMS: calcd for C₂₅H₂₀ClFN₂O₅S+H⁺, 515.08382; found (ESI, [M+H]⁺ Obs'd), 515.0838.

### Example 57

### 4-({3-[3-(8-chloroquinazolin-4-yl)-4-fluorophenoxy]phenyl}sulfonyl)butanoic acid

The title compound was prepared according to a similar procedure to that described in Example 27 but using 4-[(3-{4-fluoro-3-[8-chloroquinazolin-4-yl]phenoxy}phenyl)sulfonyl]butanamide in place of 4-[(3-{4-fluoro-3-[8-(trifluoromethyl)-quinazolin-4-yl]phenoxy}phenyl)sulfonyl]butanamide. MS (ESI) m/z 501.1; HRMS: calcd for C₂₄H₁₈ClFN₂O₅S+H⁺, 501.06817; found (ESI, [M+H]⁺ Obs'd), 501.0680.

### Example 58

### 3-[(3-{4-chloro-3-[2-methyl-8-(trifluoromethyl)quinazolin-4-yl]phenoxy}phenyl)sulfonyl]propan-1-ol

A stirred mixture of 4-chloro-3-[2-methyl-8-(trifluoromethyl)quinazolin-4-yl]phenol (250 mg, 0.74 mmol), 3-(3-bromophenylsulfonyl)propan-1-ol (309 mg, 1.11 mmol), cesium carbonate (721 mg, 2.21 mmol), copper (I) iodide (25 mg, 0.18 mmol) and N,N-dimethylglycine hydrochloride (34 mg, 0.24 mmol) were heated to 95° C. in anhydrous dioxane. After 18 h, the reaction was partitioned between ethyl acetate and water. The layers were separated and the organic layer was washed with water, dried (MgSO₄), filtered and the solvent removed, in vacuo, to give an amber oil. This material was adsorbed onto silica and purified by column chromatography eluting with a methanol in methylene chloride (0-5% MeOH in CH₂Cl₂) to afford the product as a white solid. This material contained impurities and was further purified by supercritical fluid chromatography. The product was obtained as a white solid. MS (ES) m/z [M +H]⁺537.1.

### Example 59

### 4-[(3-{4-chloro-3-[2-methyl-8-(trifluoromethyl)quinazolin-4- yl]phenoxy}phenyl)sulfonyl]-butanenitrile

### Step 1: 3-[(3-{4-chloro-3-[2-methyl-8-(trifluoromethyl)quinazolin-4- yl]phenoxy}phenyl)sulfonyl]-propyl methanesulfonate

To a solution of 3-[(3-{4-chloro-3-[2-methyl-8-(trifluoromethyl)quinazolin-4-yl]phenoxy}phenyl)sulfonyl]propan-1-ol (230 mg, 0.43 mmol) and triethylamine (1.1 mL, 7.71 mmol) in methylene chloride (10 mL), cooled in an ice bath, was added, dropwise, methanesulfonyl chloride (431 μL, 5.57 mmol) and the solution allowed to warm to room temperature and stirred for 18 h. The reaction was concentrated, in vacuo, and the material adsorbed onto silica and purified by column chromatography, eluting with a gradient of ethyl acetate in hexane (0-100% E) to afford the product as an oil. MS (ES) m/z [M+H]⁺ 615.1.

### Step 2: 4-[(3-{4-chloro-3-[2-methyl-8-(trifluoromethyl)quinazolin-4- yl]phenoxy}phenyl)sulfonyl]-butanenitrile

3-[(3-{4-chloro-3-[2-methyl-8-(trifluoromethyl)quinazolin-4-yl]phenoxy}phenyl)sulfonyl]-propyl methanesulfonate (80 mg, 0.13 mmol) and potassium cyanide (432 mg, 6.63 mmol) were stirred at room temperature in anhydrous dimethylformamide (4 mL). After 18 h, the reaction was partitioned between ethyl acetate and water. The layers were separated and the organic layer was dried (MgSO₄), filtered and the concentrated material adsorbed onto silica and purified by column chromatography, eluting with a 0-50% gradient of ethyl acetate in hexane to afford the product as a white solid. MS (ES) m/z [M+H]⁺ 546.1.

### Example 60

### 4-[(3-{4-chloro-3-[2-methyl-8-(trifluoromethyl)quinazolin-4- yl]phenoxy}phenyl)sulfonyl]-butan-1-ol

The title compound was prepared according to a similar procedure to that described in Example 58 but using 4-(3-bromophenylsulfonyl)butan-1-ol in place of 4-(3-bromophenylsulfonyl)propan-1-ol. MS (ES) m/z [M+H]⁺ 551.2.

### Example 61

### 5-[(3-{4-chloro-3-[2-methyl-8-(trifluoromethyl)quinazolin-4- yl]phenoxy}phenyl)sulfonyl]-pentanenitrile

Step 1: 4-[(3-{4-chloro-3-[2-methyl-8-(trifluoromethyl)quinazolin-4- yl]phenoxy}phenyl)sulfonyl]-butyl methanesulfonate

The title compound was prepared according to a similar procedure to that described in Example 59, Step 1 but using 4-[(3-{4-chloro-3-[2-methyl-8-(trifluoromethyl)quinazolin-4-yl]phenoxy}phenyl)sulfonyl]butan-1-ol in place of 3-[(3-{4-chloro-3-[2-methyl-8-(trifluoro-methyl)quinazolin-4- yl]phenoxy}phenyl)sulfonyl]propan-1-ol. MS (ES) m/z [M+H]⁺ 629.1.

### Step 2: 5-[(3-{4-chloro-3-[2-methyl-8-(trifluoromethyl)quinazolin-4-yl]phenoxy}phenyl)sulfonyl]pentanenitrile

The title compound was prepared according to a similar procedure to that described in Example 59, Step 2 but using 4-[(3-{4-chloro-3-[2-methyl-8-(trifluoromethyl)quinazolin-4-yl]phenoxy}phenyl)sulfonyl]butyl methanesulfonate in place of 3-[(3-{4-chloro-3-[2-methyl-8-(trifluoromethyl)quinazolin-4-yl]phenoxy}phenyl)sulfonyl]propyl methanesulfonate. MS (ES) m/z [M+H]⁺ 560.2.

### Example 62

### N-{3-[(3-{4-chloro-3-[2-methyl-8-(trifluoromethyl)quinazolin-4-yl]phenoxy}phenyl)sulfonyl]-propyl}methanesulfonamide

### Step 1:3-(3-(4-chloro-3-(2-methyl-8-(trifluoromethyl)quinazolin-4-yl)phenoxy)phenylsulfonyl)-propan-1-amine

3-(3-(4-chloro-3-(2-methyl-8-(trifluoromethyl)quinazolin-4-yl)phenoxy)phenylsulfonyl)-propan-1-amine (100 mg, 0.16 mmol) was heated to 60° C. in 7N ammonia in methanol (5 mL) and anhydrous tetrahydrofuran (3 mL) for 18 h. The solvent was removed in vacuo, and the material was adsorbed onto silica and purified by column chromatography, eluting with a 0-5% NH₃-MeOH in CH₂Cl₂ to afford a clear oil. This material contained impurities and was further purified by preparative high pressure liquid chromatography (CH₃CN/H₂O). MS (ESI) m/z [M+H⁺ 536.1; HRMS: calcd for C₂₅H₂₁ClF₃N₃O₃S+H⁺, 536.1017; found (ESI, [M+H⁺), 536.1017.

### Step 2: N-{3-[(3-{4-chloro-3-[2-methyl-8-(trifluoromethyl)quinazolin-4-yl]phenoxy}phenyl)-sulfonyl]propyl}methanesulfonamide

To a stifling solution of 3-(3-(4-chloro-3-(2-methyl-8-(trifluoromethyl)quinazolin-4-yl)phenoxy)phenylsulfonyl)propan-1-amine (35 mg, 0.065 mmol) and triethylamine (39 μL, 0.28 mmol) in methylene chloride (1 mL) was added methanesulfonyl chloride (19 μL, 0.24 mmol). After 18 h the reaction was concentrated, in vacuo, and the material adsorbed onto silica and purified by column chromatography eluting with a gradient of 0-75% ethyl acetate in hexane to afford the product as a white solid. MS (ESI) m/z [M+H]⁺ 614.1; HRMS: calcd for C₂₆H₂₃ClF₃N₃O₅S₂+H⁺, 614.0793; found (ESI, [M+H]⁺), 614.0791.

### Example 63

### N-{4-[(3-{4-chloro-3-[2-methyl-8-(trifluoromethyl)quinazolin-4- yl]phenoxy}phenyl)sulfonyl]-butyl}methanesulfonamide

The title compound was prepared according to a similar procedure to that described in Example 62, Step 2 but using N-{4-[(3-{4-chloro-3-[2-methyl-8-(trifluoromethyl)quinazolin-4-yl]phenoxy}phenyl)sulfonyl]butyl} amine in place of 3-(3-(4-chloro-3-(2-methyl-8-(trifluoro-methyl)quinazolin-4-yl)phenoxy)phenylsulfonyl)propan-1-amine. MS (ESI) m/z [M+H]⁺ 628.1; HRMS: calcd for C₂₇H₂₅ClF₃N₃O₅S₂+H⁺, 628.0949; found (ESI, [M+H]⁺), 628.0949.

### Example 64

### 4-[(3-{4-chloro-3-[2-methyl-8-(trifluoromethyl)quinazolin-4-yl]phenoxy}phenyl)sulfonyl]-butanamide

4-[(3-{4-chloro-3-[2-methyl-8-(trifluoromethyl)quinazolin-4-yl]phenoxy}phenyl)sulfonyl]-butanenitrile (50 mg, 0.092 mmol) was stirred in ammonium hydroxide (3 mL) and 30% aqueous hydrogen peroxide (3 mL). After 18 h, the reaction was partitioned between ethyl acetate and water. The organic phase was dried (MgSO₄), filtered, concentrated and adsorbed onto silica and purified by column chromatography eluting with a gradient of 0-100% ethyl acetate in hexane to afford the product as a white solid. MS (ESI) m/z [M+H]⁺ 564.1; HRMS: calcd for C₂₆H₂₁ClF₃N₃O₄S+H⁺, 564.0966; found (ESI, [M+H]⁺), 564.0967.

### Example 65

### 4-[(3-{4-chloro-3-[2-methyl-8-(trifluoromethyl)quinazolin-4-yl]phenoxy}phenyl)sulfonyl]-butanoic acid

The title compound was prepared according to a similar procedure to that described in Example 27 but using 4-[(3-{4-chloro-3-[2-methyl-8-(trifluoromethyl)quinazolin-4-yl]phenoxy}phenyl)sulfonyl]butanamide in place of 4-[(3-{4-fluoro-3-[8-(trifluoromethyl)quinazolin-4-yl]phenoxy}phenyl)sulfonyl]butanamide. MS (ESI) m/z [M+H]⁺ 565.1; HRMS: calcd for C₂₆H₂₀ClF₃N₂O₅S+H⁺, 565.0806; found (ESI, [M+H]⁺), 565.0809.

TABLE 1
The structures of the title compounds of Examples 1-11 and 14-65

| Example | Chemical Structure |
|---|---|
| 1 | 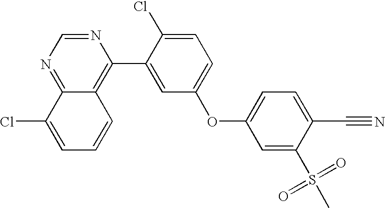 |
| 2 | 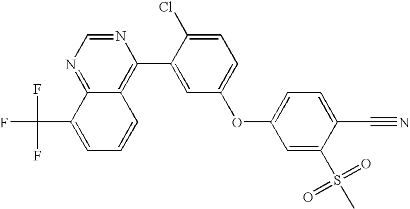 |
| 3 | 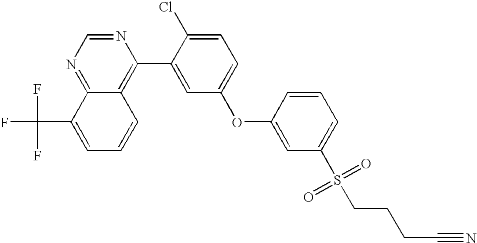 |
| 4 | 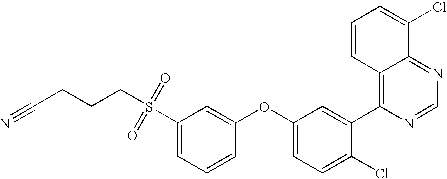 |
| 5 | 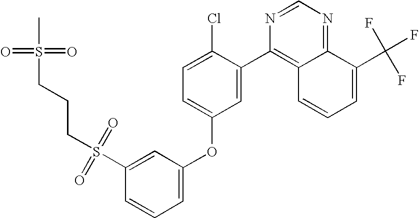 |
| 6 | 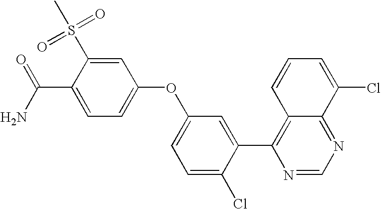 |
| 7 | 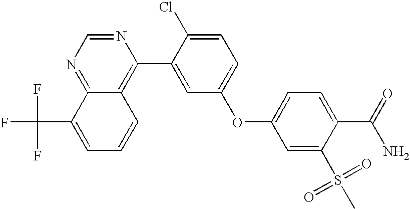 |
| 8 | 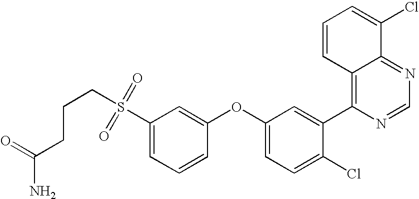 |
| 9 | 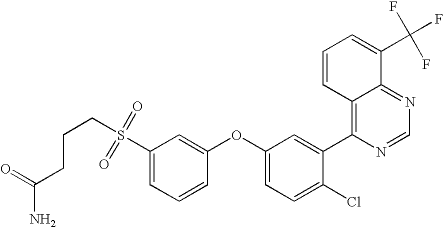 |
| 10 | 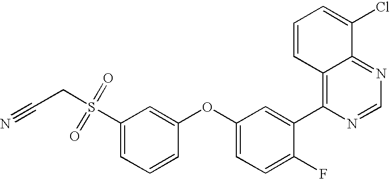 |
| 11 | 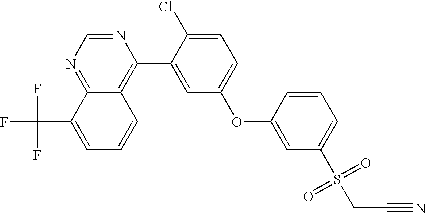 |
| 14 | 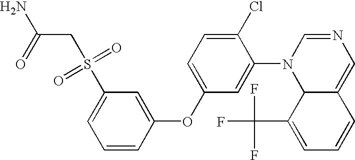 |
| 15 | 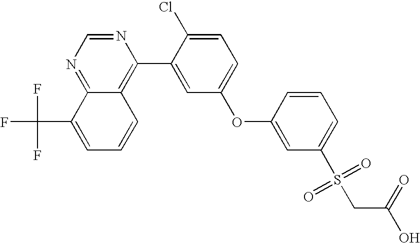 |
| 16 | 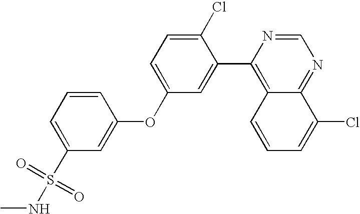 |
| 17 | 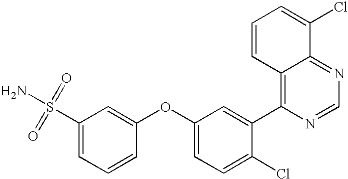 |
| 18 | 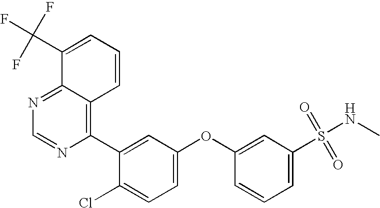 |
| 19 | 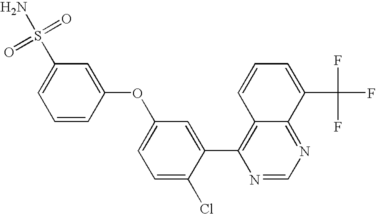 |
| 20 | 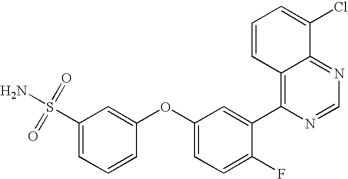 |
| 21 | 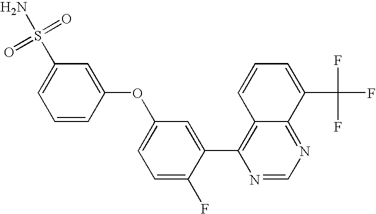 |
| 22 | 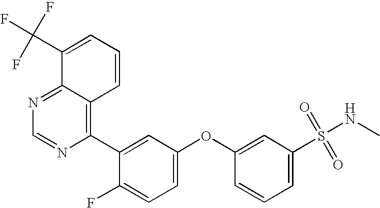 |
| 23 | 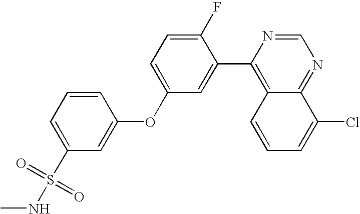 |
| 24 | 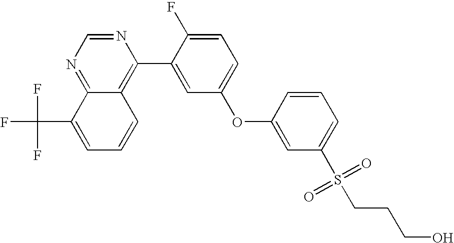 |
| 25 | 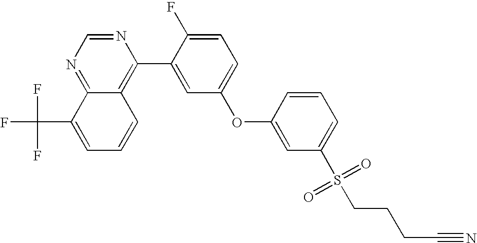 |
| 26 | 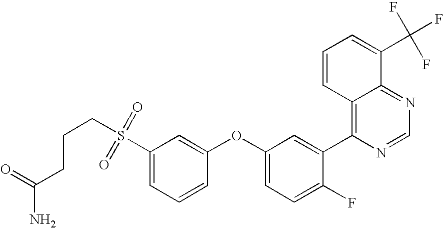 |
| 27 | 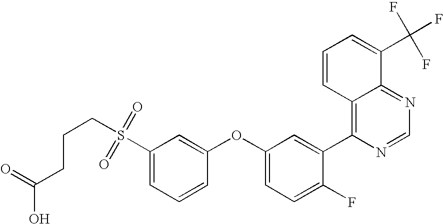 |
| 28 | 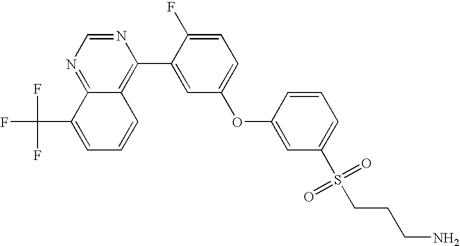 |
| 29 | 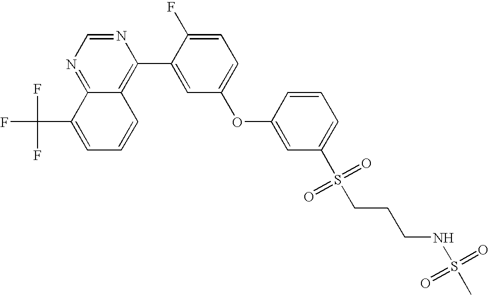 |
| 30 | 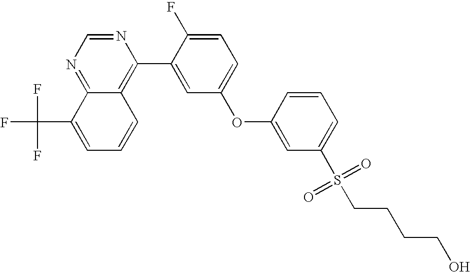 |
| 31 | 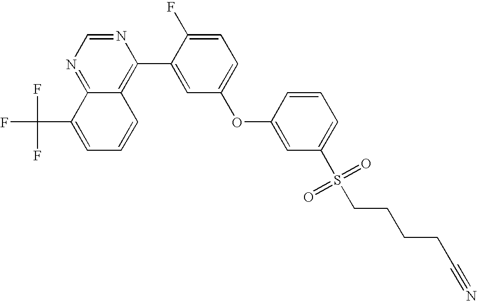 |
| 32 | 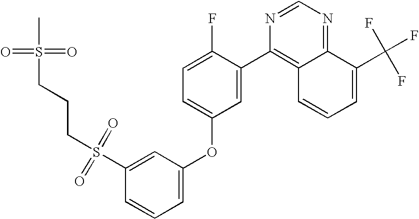 |
| 33 | 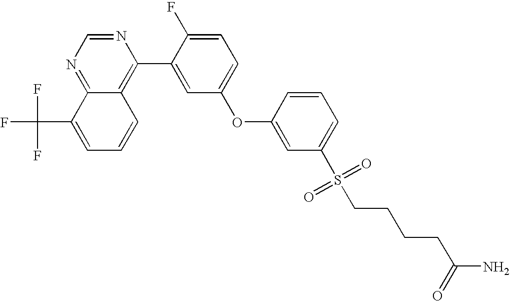 |
| 34 | 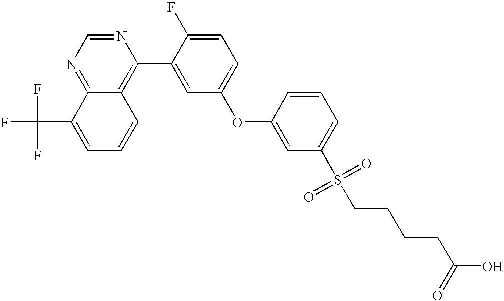 |
| 35 | 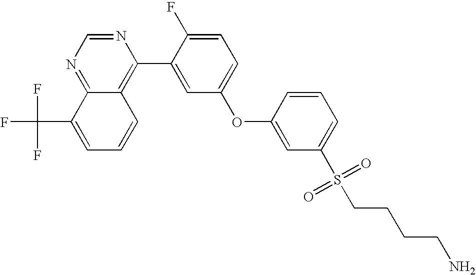 |
| 36 | 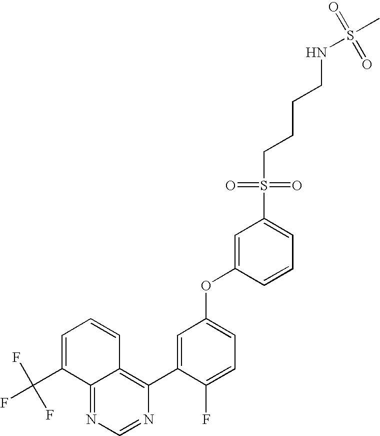 |
| 37 | 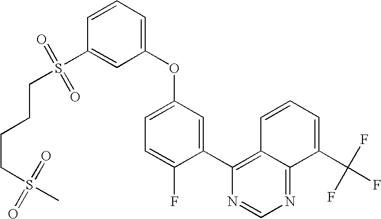 |
| 38 | 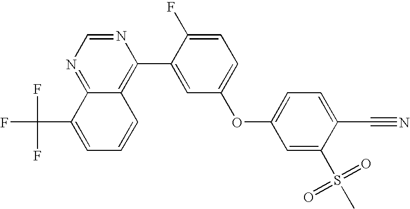 |
| 39 | 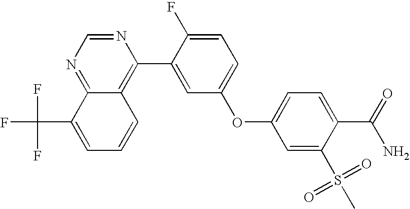 |
| 40 | 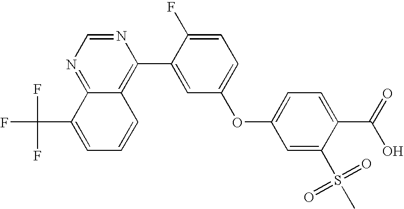 |
| 41 | 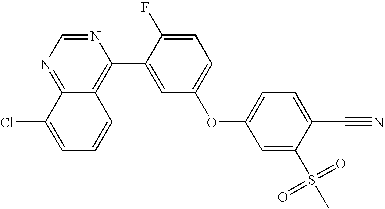 |
| 42 | 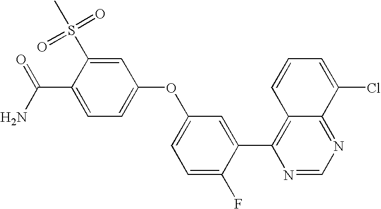 |
| 43 | 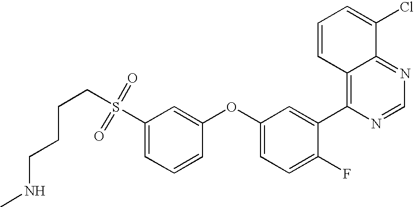 |
| 44 | 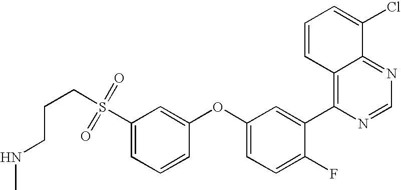 |
| 45 | 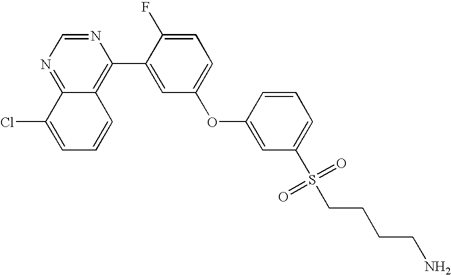 |
| 46 | 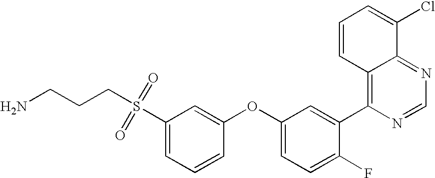 |
| 47 | 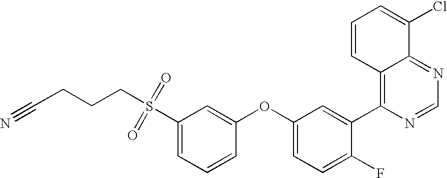 |
| 48 | 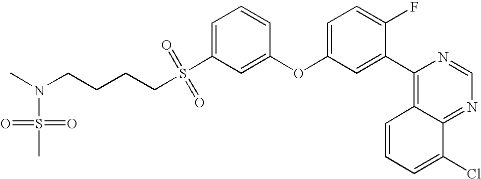 |
| 49 | 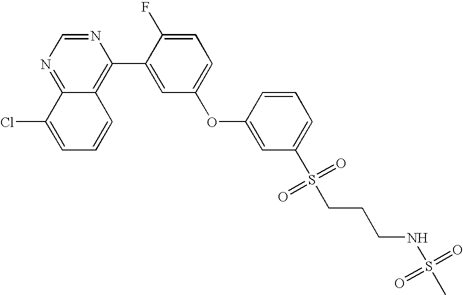 |
| 50 | 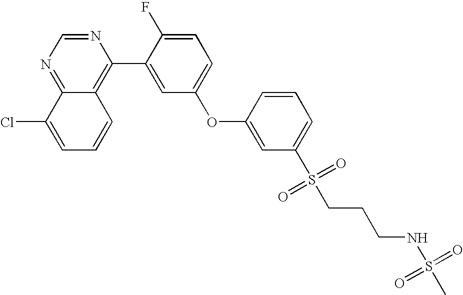 |
| 51 | 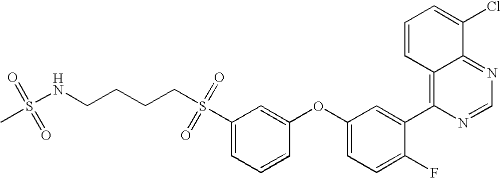 |
| 52 | 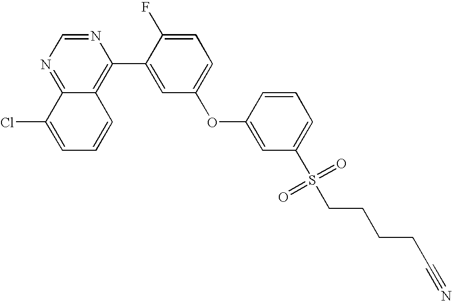 |
| 53 | 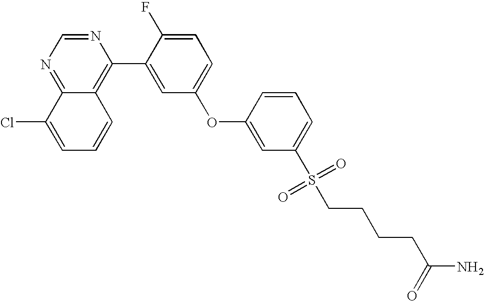 |
| 54 | 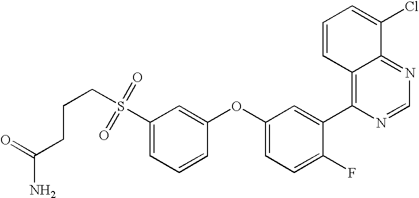 |
| 55 | 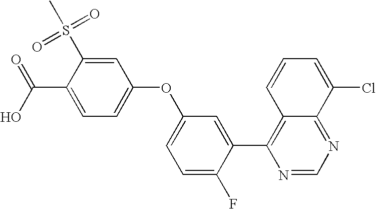 |
| 56 | 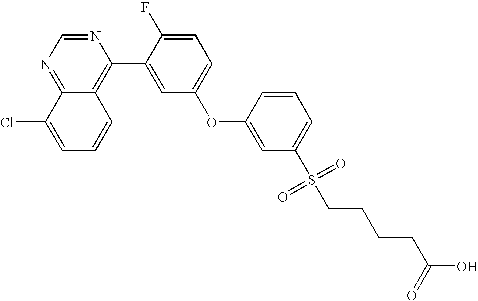 |
| 57 | 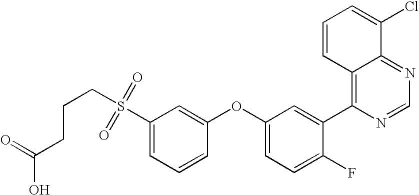 |
| 58 | 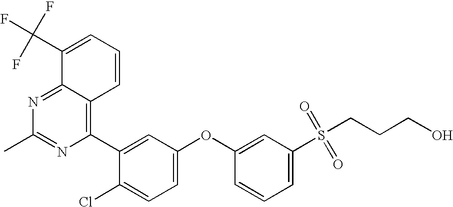 |
| 59 | 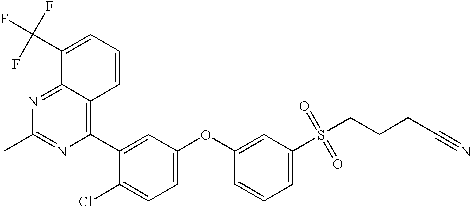 |
| 60 | 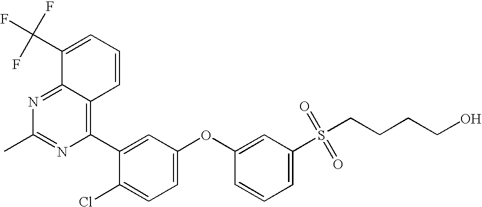 |
| 61 | 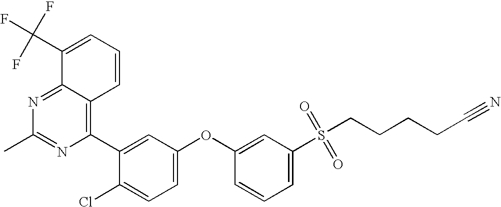 |
| 62 | 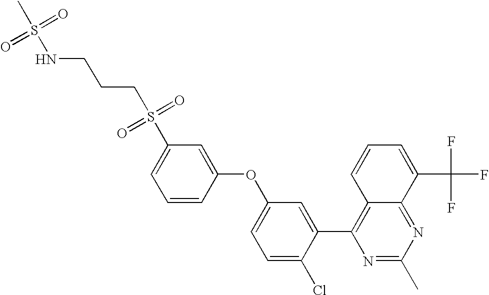 |
| 63 | 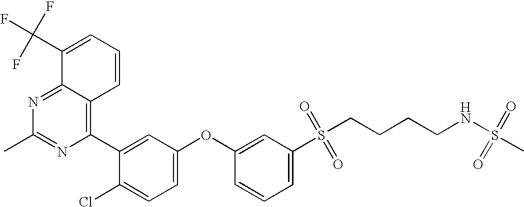 |
| 64 | 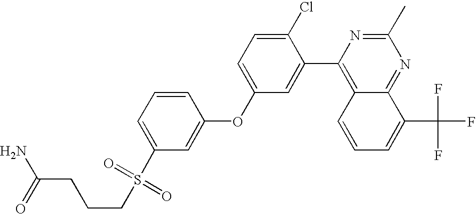 |
| 65 | 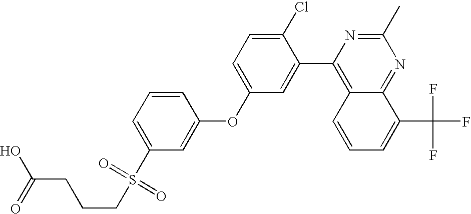 |

### Example 66

### Biological Testing

Representative compounds of this invention were evaluated in conventional pharmacological test procedures which measured their affinity to bind to LXR and to upregulate the gene ABCA1, which causes cholesterol efflux from atherogenic cells, such as macrophages.

LXR activation can be critical for maintaining cholesterol homeostasis, but its coincident regulation of fatty acid metabolism may lead to increased serum and hepatic triglyceride levels. Selective LXR modulators that activate cholesterol efflux with minimal impact on SREBP-1c expression and triglyceride synthesis in liver would be expected to reduce atherosclerotic risk with an improved therapeutic index and minimize the potential for deleterious effects on metabolic balance.

The test procedures performed, and results obtained, are briefly described in the following sections:

I. Ligand-Binding Test Procedure for Human LXRβ

II. Ligand-Binding Test Procedure for Human LXRα

III. Quantitative Analysis of ABCA1 Gene Regulation in THP-1 Cells

IV. Quantitative Analysis of SREBP-1C Gene Regulation in THP-1 Cells

V. Results

### I. Ligand-Binding Test Procedure for Human LXRβ.

Ligand-binding to the human LXRβ was demonstrated for representative compounds of this invention by the following procedure.

### Materials and Methods:

Buffer: 100 mM KCl, 100 mM TRIS (pH 7.4 at +4° C.), 8.6% glycerol, 0.1 mM PMSF*, 2 mM MTG*, 0.2% CHAPS (* not used in wash buffer)
Tracer: ³H T0901317
Receptor source: E. coli extracted from cells expressing biotinylated hLXRβ. Extract was made in a similar buffer as above, but with 50 mM TRIS.

### Day 1

Washed streptavidin and coated flash plates with wash buffer.
Diluted receptor extract to give Bₘₐₓ˜4000 cpm and add to the wells.
Wrapped the plates in aluminum foil and stored them at +4° C. overnight.

### Day 2

Made a dilution series in DMSO of the test ligands.
Made a 5 nM solution of the radioactive tracer in buffer.
Mixed 2500 diluted tracer with 50 of the test ligand from each concentration of the dilution series.
Washed the receptor-coated flash plates.
Added 2000 per well of the ligand/radiolabel mixture to the receptor-coated flash plates.
Wrapped the plates in aluminum foil and incubate at +4° C. over night.

### Day 3

Aspirated wells, and washed the flashed plates. Sealed the plate.
Measured the remaining radioactivity in the plate.

### II. Ligand-Binding Test Procedure for Human LXRa.

Ligand-binding to the human LXRα was demonstrated for representative compounds of this invention by the following procedure.

### Materials and Methods:

Buffer: 100 mM KCl, 100 mM TRIS (pH 7.4 at +4° C.), 8.6% glycerol, 0.1 mM PMSF*, 2 mM MTG*, 0.2% CHAPS (* not used in wash buffer)
Tracer: ³H T0901317
Receptor source: E. coli extract from cells expressing biotinylated hLXRα. Extract was made in a similar buffer as above, but with 50 mM TRIS.

### Day 1

Washed streptavidin and coated flash plates with wash buffer.
Diluted receptor extract to give Bmax˜4000 cpm and add to the wells.
Wrapped the plates in aluminum foil and stored them at +4° C. over night.

### Day 2

Made a dilution series in DMSO of the test ligands.
Made a 5 nM solution of the radioactive tracer in buffer.
Mixed 2500 diluted tracer with 50 of the test ligand from each concentration of the dilution series.
Washed the receptor-coated flash plates.
Added 2000 per well of the ligand/radiolabel mixture to the receptor-coated flash plates.
Wrapped the plates in aluminum foil and incubate at +4° C. over night.

### Day 3

Aspirated wells, and wash the flashed plates. Sealed the plate.
Measured the remaining radioactivity in the plate.

### III. Quantitative Analysis of ABCA1 Gene Regulation In THP-1 Cells.

The compounds of formula (I) effect on the regulation of the ABCA1 gene was evaluated using the following procedure.

### Materials and Methods

Cell culture: The THP-1 monocytic cell line (ATCC #TIB-202) was obtained from American Type Culture Collection (Manassas, Va.) and cultured in RPMI 1640 medium (Gibco, Carlsbad, Calif.) containing 10% FBS, 2 mM L-glutamine, and 55 uM beta-Mercaptoethanol (BME). Cells were plated in 96-well format at a density of 7.5×10⁴ in complete medium containing 50-100 ng/ml phorbal 12,13-dibutyrate (Sigma, St. Louis, Mo.) for three days to induce differentiation into adherent macrophages. Differentiated THP-1 cells were treated with test compounds or ligands dissolved in DMSO (Sigma, D-8779) in culture medium lacking phorbal ester. Final concentrations of DMSO did not exceed 0.3% of the media volume. Dose response effects were measured in duplicate, in the range of 0.001 to 30 micromolar concentrations and treated cells were incubated for an additional 18 hrs prior to RNA isolation. Unstimulated cells treated with vehicle were included as negative controls on each plate. An LXR agonist reference, N-(2,2,2-Trifluoro-ethyl)-N-[4-(2,2,2-trifluoro-1-hydroxy-1-trifluoromethyl-ethyl)-phenyl]-benzenesulfonamide (Schultz, Joshua R., Genes & Development (2000), 14(22), 2831-2838), was dosed at 1.0 uM and served as a positive control. In antagonist mode, the compound under study is analyzed in the presence of 150 nM GW3965, trifluoromethyl-benzyl)-(2,2-diphenyl-ethyl)-amino]-propoxyl-phenyl)-acetic acid (Collins, J. L., J. Med. Chem. (2000), 45:1963-1966.). Results of antagonist analysis are expressed as % antagonism and IC50 (in μM).
RNA isolation and quantitation: Total cellular RNA was isolated from treated cells cultured in 96-well plates using PrepStation 6100 (Applied Biosystems, Foster City, Calif.), according to the manufacturer's recommendations. RNA was resuspended in ribonuclease-free water and stored at −70° C. prior to analysis. RNA concentrations were quantitated with RiboGreen test procedure, #R-11490 (Molecular Probes, Eugene, Oreg.).

Gene expression analysis: Gene-specific mRNA quantitation was performed by real-time PCR with the Perkin Elmer Corp. chemistry on an ABI Prism 7700 Sequence detection system (Applied Biosystems, Foster City, Calif.) according to the manufacturer's instructions. Samples (50-100 ng) of total RNA were assayed in duplicate or triplicate in 50 μl reactions using one-step RT-PCR and the standard curve method to estimate specific mRNA concentrations. Sequences of gene-specific primer and probe sets were designed with Primer Express Software (Applied Biosystems, Foster City, Calif.). The human ABCA1 primer and probe sequences are: forward, CAACATGAATGCCATTTTCCAA, reverse, ATAATCCCCTGAACCCAAGGA, and probe, 6FAM-TAAAGCCATGCCCTCTGCAGGAACA-TAMRA. RT and PCR reactions were performed according to PE Applied Biosystem's protocol for Taqman Gold RT-PCR or Qiagen's protocol for Quantitect probe RT-PCR. Relative levels of ABCA1 mRNA are normalized using GAPDH mRNA or 18S rRNA probe/primer sets purchased commercially (Applied Biosystems, Foster City, Calif.).

### Statistics:

Mean, standard deviation and statistical significance of duplicate evaluations of RNA samples were assessed using ANOVA, one-way analysis of variance using SAS analysis.

### Reagents:

GAPDH Probe and Primers—Taqman GAPDH Control Reagents 402869 or 4310884E 18S Ribosomal RNA—Taqman 18S Control Reagents 4308329
10 Pack Taqman PCR Core Reagent Kit 402930
Qiagen Quantitect probe RT-PCR 204443.

### IV. Quantitative Analysis of SREBP-1C Gene Regulation in THP-1 Cells

The compounds of formula (II) effect on the regulation of the SREBP-1c gene was evaluated using the same procedure as described for ABCA1 however, a primer and probe set specific for human SREBP-1c was substituted in gene expression analysis. The human SREBP-1c primer and probe sequences are: forward, AGGGCGGGCGCAGAT, reverse, GGTTGTTGATAAGCTGAAGCATGT, and probe, 6FAM-TCGAAAGTGCAATCCATGGCTCCG-TAMRA.

### V. Results:

TABLE 2
TPSA values for Examples 1-11, 14-
23, 25-29, 31-57, 59 and 61-65

| Example | TPSA |
|---|---|
| 1 | 93 |
| 2 | 93 |
| 3 | 93 |
| 4 | 93 |
| 5 | 103 |
| 6 | 112 |
| 7 | 112 |
| 8 | 112 |
| 9 | 112 |
| 10 | 93 |
| 11 | 93 |
| 14 | 112 |
| 15 | 106 |
| 16 | 81 |
| 17 | 95 |
| 18 | 81 |
| 19 | 95 |
| 20 | 95 |
| 21 | 95 |
| 22 | 81 |
| 23 | 82 |
| 25 | 93 |
| 26 | 112 |
| 27 | 106 |
| 28 | 95 |
| 29 | 115 |
| 31 | 93 |
| 32 | 103 |
| 33 | 112 |
| 34 | 106 |
| 35 | 95 |
| 36 | 115 |
| 37 | 103 |
| 38 | 93 |
| 39 | 112 |
| 40 | 106 |
| 41 | 93 |
| 42 | 112 |
| 43 | 81 |
| 44 | 81 |
| 45 | 95 |
| 46 | 95 |
| 47 | 93 |
| 48 | 107 |
| 49 | 107 |
| 50 | 115 |
| 51 | 115 |
| 52 | 93 |
| 53 | 112 |
| 54 | 112 |
| 55 | 106 |
| 56 | 106 |
| 57 | 106 |
| 59 | 93 |
| 61 | 93 |
| 62 | 115 |
| 63 | 115 |
| 64 | 112 |
| 65 | 106 |

TABLE 3

| | hLXRb binding | hLXRa binding |
|---|---|---|
| Example | IC50 (uM) | IC50 (uM) |
| 1 | 0.329 | 3.128 |
| 2 | 0.152 | 1.065 |
| 3 | 0.035 | 0.512 |
| 4 | 0.105 | 1.807 |
| 5 | 0.140 | 0.889 |
| 6 | 1.264 | 3.842 |
| 7 | 0.288 | 0.986 |
| 8 | 0.126 | 1.313 |
| 9 | 0.041 | 0.407 |
| 10 | 0.286 | 1.531 |
| 11 | 0.040 | 0.425 |
| 14 | 0.056 | 0.705 |
| 15 | 0.019 | 1.012 |
| 16 | 0.011 | 0.146 |
| 17 | 0.151 | 1.709 |
| 18 | 0.004 | 0.081 |
| 19 | 0.049 | 0.581 |
| 20 | 0.336 | 2.880 |
| 21 | 0.121 | 1.005 |
| 22 | 0.010 | 0.149 |
| 23 | 0.017 | 0.033 |
| 25 | 0.112 | 1.522 |
| 26 | 0.184 | 2.478 |
| 27 | 1.198 | 16.661 |
| 28 | 0.116 | 2.989 |
| 29 | 0.157 | 1.329 |
| 31 | 0.052 | 0.628 |
| 32 | 0.059 | 0.660 |
| 33 | 0.130 | 1.352 |
| 34 | 1.433 | 11.912 |
| 35 | 0.584 | 9.422 |
| 36 | 0.221 | 2.731 |
| 37 | 0.066 | 0.536 |
| 38 | 0.506 | 4.096 |
| 39 | 1.208 | 4.367 |
| 41 | 1.061 | |
| 42 | 4.901 | 10.906 |
| 43 | 3.586 | |
| 44 | 0.965 | 7.306 |
| 45 | 1.880 | 11.453 |
| 46 | 0.296 | 8.183 |
| 47 | 0.274 | 3.984 |
| 48 | 1.019 | |
| 49 | 1.987 | |
| 50 | 0.471 | 6.348 |
| 51 | 0.444 | 4.891 |
| 52 | 0.107 | 2.026 |
| 53 | 0.324 | 231.859 |
| 54 | 0.584 | 6.544 |
| 56 | 3.427 | |
| 57 | 4.208 | |
| 59 | 0.150 | 2.258 |
| 61 | 0.097 | 2.798 |
| 62 | 0.343 | 7.043 |
| 63 | 0.326 | 4.543 |

TABLE 4

Gene regulation by LXR
(murine)

| | EC50 ABCA1 | Agonism ABCA1 |
|---|---|---|
| Example | (uM) | (%) |
| 3 | 2.705 | 71 |
| 4 | 4.340 | 48 |
| 5 | 3.245 | 51 |
| 8 | 2.170 | 74 |
| 9 | 1.900 | 91 |
| 11 | 1.755 | 95 |
| 14 | 1.375 | 95 |
| 15 | 2.920 | 51 |
| 16 | 0.255 | 98 |
| 17 | 1.480 | 74 |
| 18 | 0.085 | 94 |
| 19 | 0.220 | 95 |
| 21 | 0.650 | 68 |
| 22 | 0.290 | 81 |
| 23 | 0.450 | 85 |
| 25 | 2.470 | 68 |
| 28 | | 3 |
| 31 | 1.765 | 99 |
| 32 | 1.015 | 78 |
| 37 | 0.415 | 77 |
| 52 | 1.800 | 74 |
| 61 | | 27 |

TABLE 5
Brain penetration data

| | | | Brain/ |
|---|---|---|---|
| | | | Blood |
| Entry | Compound | TPSA^{#} | ratio |
| 1* | 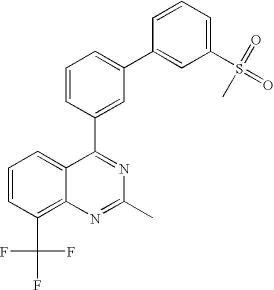 | 60 | 2.27 |
| 2* | 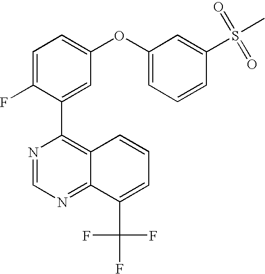 | 78 | 1.45 |
| 3* | 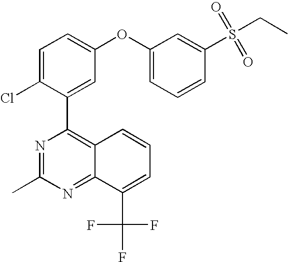 | 78 | 1.57 |
| 4* | 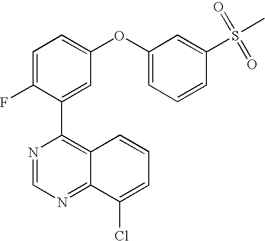 | 69 | 1.20 |
| 5* | 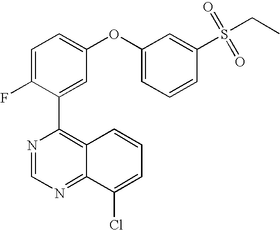 | 69 | 1.20 |
| 6 | Example 25 | 93 | 0.55 |
| 7 | Example 11 | 93 | 0.16 |
| 8 | Example 9 | 112 | 0.06 |

*The compounds delineated in entries 1-5 are disclosed in PCT/US2008/063685, which was filed on May 15, 2008.
^{#}Data obtained from Table 2.

Based on the results obtained in the standard pharmacological test procedures, the compounds of this invention can be useful in treating or inhibiting LXR mediated diseases. In particular, the compounds of this invention can be useful in the treatment or prevention of atherosclerosis and atherosclerotic lesions, lowering LDL cholesterol levels, increasing HDL cholesterol levels, increasing reverse cholesterol transport, inhibiting cholesterol absorption, treatment or inhibition of cardiovascular diseases (e.g., acute coronary syndrome, restenosis, coronary artery disease), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

A number of embodiments of the invention have been described. Nevertheless, it will be understood that various modifications may be made without departing from the spirit and scope of the invention. Accordingly, other embodiments are in the claims.

What is claimed is:

1. A compound having formula (I): 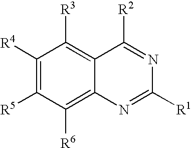
wherein:
R¹ is hydrogen or C₁-C₂ alkyl;
R² has the following formula: 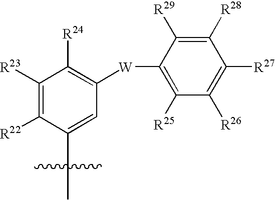
wherein:
each of R²², R²³, and R²⁴ is, independently, hydrogen or R^{a};
W is a bond, —O—, —NH—, —N(C₁-C₃ alkyl)-, C₁₋₂ alkylene, C₂ alkenylene, C₂ alkynylene, —(C₁₋₂ alkylene)O—, or —O(C₁₋₂ alkylene)-;
wherein all of R²⁵, R²⁶, R²⁷, R²⁸, and R²⁹ are defined according to either (A) or (B) below:
(A):
one of R²⁵, R²⁶, R²⁷, R²⁸, and R²⁹ is —W¹—S(O)ₙR^{b} or —W¹—S(O)ₙNR^{c}R^{d}; and the others are each, independently, hydrogen or R^{e};
or
(B):
(a) one of R²⁵, R²⁶, R²⁷, R²⁸, and R²⁹ is —W¹—S(O)ₙR^{f}; and
(b) one of R²⁵, R²⁶, R²⁷, R²⁸, and R²⁹ is —W¹—C(O)OR^{g}, —W¹—C(O)NR^{h}Rⁱ; or —W²—CN; and
(c) the others are each, independently, hydrogen or R^{e};
each of R³, R⁴ and R⁵ is, independently:
(i) hydrogen; or
(ii) halo; or
(iii) C₁-C₃ alkyl or C₁-C₃ haloalkyl, each of which is optionally substituted with from 1-2 R^{j};
R⁶ is:
(i) halo; or
(ii) C₁-C₆ alkyl or C₁-C₆ haloalkyl, each of which is optionally substituted with from 1-3 R^{j}; or
(iii) nitro; hydroxy; C₁-C₆ alkoxy; C₁-C₆ haloalkoxy; C₁-C₆ thioalkoxy; C₁-C₆ thiohaloalkoxy; cyano; or S(O)ₙR^{f};
wherein:
W¹ is a bond, —NH—, —N(C₁-C₃ alkyl)-, or C₁₋₂ alkylene;
W² is a bond or C₁₋₂ alkylene;
R^{a} at each occurrence is, independently, C₁-C₃ alkyl; C₁-C₃ haloalkyl; halo; hydroxy; NR^{m}Rⁿ; C₁-C₃ alkoxy; or C₁-C₃ haloalkoxy;
R^{b} is C₁-C₆ alkyl or C₁-C₆ haloalkyl, each of which is substituted with from 1-2 R^{o};
each of R^{c} and R^{d} at each occurrence is, independently:
(i) hydrogen; or
(i) C₁-C₆ alkyl or C₁-C₆ haloalkyl, each of which is optionally substituted with from 1-3 R^{j}; or
(ii) C₂-C₆ alkenyl or C₂-C₆ alkynyl, each of which is optionally substituted with from 1-3 R^{p}; or
(iii) C₃-C₈ cycloalkyl, C₃-C₈ cycloalkenyl, heterocyclyl including 3-8 atoms, heterocycloalkenyl including 3-10 atoms, C₇-C₁₁ aralkyl, or heteroaralkyl including 6-11 atoms, each of which is optionally substituted with from 1-3 R^{q}; or
(iv) C₆-C₁₀ aryl or heteroaryl including 5-10 atoms, each of which is optionally substituted with from 1-3 R^{r}; or
R^{c} and R^{d} together with the nitrogen atom to which they are attached form a heterocyclyl including 3-8 atoms or a heterocycloalkenyl including 3-10 atoms, each of which is optionally substituted with from 1-3 R^{q};
R^{e} at each occurrence is, independently, C₁-C₃ alkyl; C₁-C₃ haloalkyl; halo; hydroxy; NR^{m}Rⁿ; C₁-C₃ alkoxy; or C₁-C₃ haloalkoxy;
R^{f} at each occurrence is, independently C₁-C₃ alkyl or C₁-C₃ haloalkyl;
R^{g} is:
(i) hydrogen; or
(i) C₁-C₆ alkyl or C₁-C₆ haloalkyl, each of which is optionally substituted with from 1-3 R^{j};
each of R^{h} and Rⁱ at each occurrence is, independently:
(i) hydrogen; or
(i) C₁-C₃ alkyl or C₁-C₃ haloalkyl, each of which is optionally substituted with from 1-2 R^{j};
R^{j} at each occurrence is, independently, NR^{m}Rⁿ; hydroxy; C₁-C₆ alkoxy or C₁-C₆ haloalkoxy;
each of R^{m} and Rⁿ at each occurrence is, independently, hydrogen, C₁-C₆ alkyl, or C₁-C₆ haloalkyl;
R^{o} at each occurrence is, independently:
(i) —C(O)NR^{t}R^{u}; or
(ii) —C(O)OR^{s}; or
(iii) —NHS(O)ₙR^{f} or —N(C₁-C₃ alkyl)S(O)ₙR^{f}; or
(iv) —S(O)ₙR^{f}; or
(v) —CN; or
(vi) —NR^{m}Rⁿ;
R^{p} at each occurrence is, independently:
(i) halo; NR^{m}Rⁿ; hydroxy; C₁-C₆ alkoxy or C₁-C₆ haloalkoxy; or
(ii) C₃-C₇ cycloalkyl optionally substituted with from 1-3 substituents independently selected from NR^{m}Rⁿ; hydroxy; C₁-C₆ alkyl, C₁-C₆ alkoxy and C₁-C₆ haloalkoxy;
R^{q} at each occurrence is, independently:
(i) halo; NR^{m}Rⁿ; hydroxy; C₁-C₆ alkoxy or C₁-C₆ haloalkoxy; or
(ii) C₁-C₆ alkyl or C₁-C₆ haloalkyl; or
(iii) C₂-C₆ alkenyl or C₂-C₆ alkynyl;
R^{r} at each occurrence is, independently:
(i) halo; NR^{m}Rⁿ; hydroxy; C₁-C₆ alkoxy or C₁-C₆ haloalkoxy; or cyano; or
(ii) C₁-C₆ alkyl or C₁-C₆ haloalkyl, each of which is optionally substituted with from 1-3 R^{j}; or
(iii) C₂-C₆ alkenyl or C₂-C₆ alkynyl, each of which is optionally substituted with from 1-3 R^{p};
R^{s} is:
(i) hydrogen; or
(i) C₁-C₆ alkyl or C₁-C₆ haloalkyl, each of which is optionally substituted with from 1-3 R^{j};
each of R^{t} and R^{u} at each occurrence is, independently:
(i) hydrogen; or
(i) C₁-C₃ alkyl or C₁-C₃ haloalkyl, each of which is optionally substituted with from 1-2 R^{j}; and
n is 1 or 2;
or an N-oxide and/or a pharmaceutically acceptable salt thereof.

2. The compound of claim 1, wherein one of R²⁵, R²⁶, R²⁷, R²⁸, and R²⁹ is —W¹—S(O)ₙR^{b} or —W¹—S(O)ₙNR^{c}R^{d}; and the others are each, independently, hydrogen or R^{e}.

3. The compound of claim 1, wherein one of R²⁵, R²⁶, R²⁷, R²⁸, and R²⁹ is —W¹—S(O)ₙR^{b}; and the others are each, independently, hydrogen or R^{e}.

4. The compound of claim 3, wherein W¹ is a bond; n is 2; and R^{b} is C₁-C₆ alkyl, which is substituted with from 1-2 R^{o}.

5. The compound of claim 4, wherein:
R^{b} is C₁-C₆ alkyl, which is substituted with 1 R^{o}; and
R^{o} is:
(i) —C(O)NR^{t}R^{u}; or
(ii) —C(O)OR^{s}; or
(iii) —NHS(O)ₙR^{f} or —N(C₁-C₃ alkyl)S(O)ₙR^{f}; or
(iv) —S(O)ₙR^{f}.

6. The compound of claim 5, wherein R^{o} is —C(O)NH₂.

7. The compound of claim 5, wherein R^{o} is —C(O)OH.

8. The compound of claim 5, wherein R^{o} is —NHSO₂CH₃ or —N(CH₃)SO₂CH₃.

9. The compound of claim 5, wherein R^{o} is —SO₂CH₃.

10. The compound of claim 5, wherein R^{b} is C₁ alkyl, which is substituted with 1 R^{o}.

11. The compound of claim 10, wherein R^{o} is —C(O)NH₂.

12. The compound of claim 10, wherein R^{o} is —C(O)OH.

13. The compound of claim 5, wherein R^{b} is C₂-C₅ alkyl, which is substituted with 1 R^{o}.

14. The compound of claim 13, wherein R^{b} is C₃-C₄ alkyl, which is substituted with 1 R^{o}.

15. The compound of claim 14, wherein R^{o} is —C(O)NH₂.

16. The compound of claim 14, wherein R^{o} is —C(O)OH.

17. The compound of claim 14, wherein R^{o} is —NHSO₂CH₃ or —N(CH₃)SO₂CH₃.

18. The compound of claim 14, wherein R^{o} is —SO₂CH₃.

19. The compound of claim 3, wherein R²⁶ is —W¹—S(O)ₙR^{b}.

20. The compound of claim 19, wherein W¹ is a bond; n is 2; and R^{b} is C₁-C₆ alkyl, which is substituted with from 1-2 R^{o}.

21. The compound of claim 20, wherein:
R^{b} is C₁-C₆ alkyl, which is substituted with 1 R^{o}; and
R^{o} is:
(i) —C(O)NR^{t}R^{u}; or
(ii) —C(O)OR^{s}; or
(iii) —NHS(O)ₙR^{f} or —N(C₁-C₃ alkyl)S(O)ₙR^{f}; or
(iv) —S(O)ₙR^{f}.

22. The compound of claim 21, wherein R^{o} is —C(O)NH₂.

23. The compound of claim 21, wherein R^{o} is —C(O)OH.

24. The compound of claim 21, wherein R^{o} is —NHSO₂CH₃ or —N(CH₃)SO₂CH₃.

25. The compound of claim 21, wherein R^{o} is —SO₂CH₃.

26. The compound of claim 21, wherein R^{b} is C₁ alkyl, which is substituted with 1 R^{o}.

27. The compound of claim 26, wherein R^{o} is —C(O)NH₂.

28. The compound of claim 26, wherein R^{o} is —C(O)OH.

29. The compound of claim 21, wherein R^{b} is C₃-C₄ alkyl, which is substituted with 1 R^{o}.

30. The compound of claim 29, wherein R^{o} is —C(O)NH₂.

31. The compound of claim 29, wherein R^{o} is —C(O)OH.

32. The compound of claim 29, wherein R^{o} is —NHSO₂CH₃ or —N(CH₃)SO₂CH₃.

33. The compound of claim 29, wherein R^{o} is —SO₂CH₃.

34. The compound of claim 21, wherein each of R²⁵, R²⁷, R²⁸, and R²⁹ is hydrogen.

35. The compound of claim 5, wherein R¹ is hydrogen.

36. The compound of claim 5, wherein one of R²², R²³, and R²⁴ is R^{a}, and the other two are hydrogen.

37. The compound of claim 36, wherein R²² is R^{a}, and each of R²³ and R²⁴ is hydrogen.

38. The compound of claim 37, wherein R²² is halo.

39. The compound of claim 38, wherein R²² is chloro or fluoro.

40. The compound of claim 5, wherein W is —O—.

41. The compound of claim 5, wherein each of R³, R⁴ and R⁵ is hydrogen.

42. The compound of claim 5, wherein R⁶ is C₁-C₄ haloalkyl.

43. The compound of claim 42, wherein R⁶ is C₁-C₄ perfluoroalkyl.

44. The compound of claim 43, wherein R⁶ is CF₃.

45. The compound of claim 5, wherein R⁶ is halo.

46. The compound of claim 45, wherein R⁶ is chloro.

47. The compound of claim 1, wherein:
(a) one of R²⁵, R²⁶, R²⁷, R²⁸, and R²⁹ is —W¹—S(O)ₙR^{f}; and
(b) one of R²⁵, R²⁶, R²⁷, R²⁸, and R²⁹ is —W¹—C(O)OR^{g}, —W¹—C(O)NR^{h}Rⁱ; or —W²—CN; and
(c) the others are each, independently, hydrogen or R^{e}.

48. The compound of claim 1, wherein:
(a) one of R²⁵, R²⁶, R²⁷, R²⁸, and R²⁹ is —W¹—S(O)ₙR^{f}; and
(b) one of R²⁵, R²⁶, R²⁷, R²⁸, and R²⁹ is —W¹—C(O)OR^{g} or —W¹—C(O)NR^{h}Rⁱ; and
(c) the others are each, independently, hydrogen or R^{e}.

49. The compound of claim 48, wherein W¹ at each occurrence is a bond.

50. The compound of claim 49, wherein:
(a) one of R²⁵, R²⁶, R²⁷, R²⁸, and R²⁹ is —SO₂CH₃; and
(b) one of R²⁵, R²⁶, R²⁷, R²⁸, and R²⁹ is —C(O)OH or —C(O)NH₂; and
(c) the others are each, independently, hydrogen or R^{e}.

51. The compound of claim 48, wherein:
(a) R²⁶ is —W¹—S(O)ₙR^{f}; and
(b) R²⁷ is —W¹—C(O)OR^{g} or —W¹—C(O)NR^{h}Rⁱ; and
(c) each of R²⁵, R²⁸, and R²⁹ is, independently, hydrogen or R^{e}.

52. The compound of claim 51, wherein R²⁶ is —SO₂R^{f}.

53. The compound of claim 52, wherein R²⁶ is —SO₂CH₃.

54. The compound of claim 51, wherein R²⁷ is —C(O)OR^{g} or —C(O)NR^{h}Rⁱ.

55. The compound of claim 54, wherein R²⁷ is —C(O)OH or —C(O)NH₂.

56. The compound of claim 51, wherein R²⁶ is —SO₂R^{f}, and R²⁷ is —C(O)OR^{g} or —C(O)NR^{h}Rⁱ.

57. The compound of claim 56, wherein R²⁶ is —SO₂CH₃, and R²⁷ is —C(O)OH or —C(O)NH₂.

58. The compound of claim 51, wherein each of R²⁵, R²⁸, and R²⁹ is hydrogen.

59. The compound of claim 1, wherein the compound has a TPSA of from about 100 Å² to about 120 Å².

60. The compound of claim 59, wherein the compound has a TPSA of from about 100 Å² to about 109 Å².

61. The compound of claim 59, wherein the compound has a TPSA of from about 110 Å² to about 120 Å².

62. The compound of claim 1, wherein the compound has a TPSA of from about 90 Å² to about 99 Å².

63. The compound of claim 1, wherein the compound is selected from the title compounds of Examples 1-11, 14-23, 25-29, 31-57, 59 and 61-65; or a pharmaceutically acceptable salt and/or N-oxide thereof.

64. A composition comprising a compound of claim 1 or a pharmaceutically acceptable salt thereof and a pharmaceutically acceptable carrier.

65. A method of preventing or treating a Liver X receptor-mediated disease or disorder, the method comprising administering to a subject in need of such treatment an effective amount of a compound of claim 1 or a pharmaceutically acceptable salt thereof.

66. A method of preventing or treating atherosclerosis, the method comprising administering to a subject in need of such treatment an effective amount of a compound of claim 1 or a pharmaceutically acceptable salt thereof.

67. A method of preventing or treating a cardiovascular disease, the method comprising administering to a subject in need of such treatment an effective amount of a compound of claim 1 or a pharmaceutically acceptable salt thereof.

68. The method of claim 67, wherein the cardiovascular disease is acute coronary syndrome or restenosis.

69. The method of claim 67, wherein the cardiovascular disease is coronary artery disease.

70. A method of preventing or treating Syndrome X, the method comprising administering to a subject in need of such treatment an effective amount of a compound of claim 1 or a pharmaceutically acceptable salt thereof.

71. A method of preventing or treating obesity, the method comprising administering to a subject in need of such treatment an effective amount of a compound of claim 1 or a pharmaceutically acceptable salt thereof.

72. A method of preventing or treating one or more lipid disorders selected from dyslipidemia, hyperlipidemia, hypertriglyceridemia, hypercholesterolemia, low HDL and/or high LDL, the method comprising administering to a subject in need of such treatment an effective amount of a compound of claim 1 or a pharmaceutically acceptable salt thereof.

73. A method of preventing or treating type I or type II diabetes, the method comprising administering to a subject in need of such treatment an effective amount of a compound of claim 1 or a pharmaceutically acceptable salt thereof.

74. A method of preventing or treating an inflammatory disease, the method comprising administering to a subject in need of such treatment an effective amount of a compound of claim 1 or a pharmaceutically acceptable salt thereof.

75. The method of claim 74, wherein the inflammatory disease is rheumatoid arthritis.

76. A method of treating a connective tissue disease, the method comprising administering to a mammal in need thereof an effective amount of a compound of claim 1 or a pharmaceutically acceptable salt thereof.

77. The method of claim 76, wherein the compound of formula (I) inhibits cartilage degradation and induces cartilage regeneration.

78. The method of claim 77, wherein the compound of formula (I) inhibits aggrecanase activity.

79. The method of claim 77, wherein the compound of formula (I) inhibits elaboration of pro-inflammatory cytokines in osteoarthritic lesions.

80. The method of claim 76, wherein the connective tissue disease is osteoarthritis or tendonitis.

81. The method of claim 76, wherein the mammal is a human.

82. A method of treating skin aging, the method comprising administering to a mammal in need thereof an effective amount of a compound of claim 1 or a pharmaceutically acceptable salt thereof.

83. The method of claim 82, wherein the mammal is a human.

84. The method of claim 82, wherein the compound of formula (I) is topically administered.

85. The method of claim 82, wherein the skin aging is derived from chronological aging, photoaging, steroid-induced skin thinning, or a combination thereof.


SEQUENCE LISTING (6 sequences)

SEQ ID NO: 1 - DNA, 22 bp, Artificial
  Synthetic Forward Primer
caacatgaatgccattttccaa

SEQ ID NO: 2 - DNA, 21 bp, Artificial
  Synthetic Reverse Primer
ataatcccctgaacccaagga

SEQ ID NO: 3 - DNA, 25 bp, Artificial
  Synthetic Probe
taaagccatgccctctgcaggaaca

SEQ ID NO: 4 - DNA, 15 bp, Artificial
  Synthetic Forward Primer
agggcgggcgcagat

SEQ ID NO: 5 - DNA, 24 bp, Artificial
  Synthetic Reverse Primer
ggttgttgataagctgaagcatgt

SEQ ID NO: 6 - DNA, 24 bp, Artificial
  Synthetic Probe
tcgaaagtgcaatccatggctccg